SEQUENCE LISTING (1372 sequences)

SEQ ID NO: 1 - protein, 12 aa, Homo sapiens
ARDSSSWYYFDY

SEQ ID NO: 2 - protein, 12 aa, Homo sapiens
ARDSSGWYYFDY

SEQ ID NO: 3 - protein, 7 aa, Homo sapiens
ARDAFDI

SEQ ID NO: 4 - protein, 9 aa, Homo sapiens
ARDSSSFDY

SEQ ID NO: 5 - protein, 16 aa, Homo sapiens
ARGYCSSTSCYDAFDI

SEQ ID NO: 6 - protein, 12 aa, Homo sapiens
ARGYSSSWYFDY

SEQ ID NO: 7 - protein, 10 aa, Homo sapiens
AREYSSSFDY

SEQ ID NO: 8 - protein, 14 aa, Homo sapiens
ARVGYSSSWYYFDY

SEQ ID NO: 9 - protein, 6 aa, Homo sapiens
ARGLDY

SEQ ID NO: 10 - protein, 8 aa, Homo sapiens
ARGDAFDI

SEQ ID NO: 11 - protein, 11 aa, Homo sapiens
ARGYSSSWYDY

SEQ ID NO: 12 - protein, 9 aa, Homo sapiens
ARGSSSFDY

SEQ ID NO: 13 - protein, 15 aa, Homo sapiens
ARGYCSGGSCYYFDY

SEQ ID NO: 14 - protein, 5 aa, Homo sapiens
ARGDY

SEQ ID NO: 15 - protein, 10 aa, Homo sapiens
ARGSGSYFDY

SEQ ID NO: 16 - protein, 13 aa, Homo sapiens
ARVYSSSWYYFDY

SEQ ID NO: 17 - protein, 6 aa, Homo sapiens
ARGVDY

SEQ ID NO: 18 - protein, 6 aa, Homo sapiens
ARDPDY

SEQ ID NO: 19 - protein, 11 aa, Homo sapiens
ARGYSGYDFDY

SEQ ID NO: 20 - protein, 11 aa, Homo sapiens
ARDSSSWYFDY

SEQ ID NO: 21 - protein, 10 aa, Homo sapiens
ARDSSSSFDY

SEQ ID NO: 22 - protein, 13 aa, Homo sapiens
ARGYSSSWYYFDY

SEQ ID NO: 23 - protein, 6 aa, Homo sapiens
ARDLDY

SEQ ID NO: 24 - protein, 11 aa, Homo sapiens
ARDSGSYYFDY

SEQ ID NO: 25 - protein, 12 aa, Homo sapiens
ARYSSSWYYFDY

SEQ ID NO: 26 - protein, 10 aa, Homo sapiens
ARDSGSYFDY

SEQ ID NO: 27 - protein, 13 aa, Homo sapiens
ARGYSSSWYWFDP

SEQ ID NO: 28 - protein, 9 aa, Homo sapiens
ARGGSYFDY

SEQ ID NO: 29 - protein, 13 aa, Homo sapiens
ARDHSSSWYYFDY

SEQ ID NO: 30 - protein, 13 aa, Homo sapiens
ARAYSSSWYYFDY

SEQ ID NO: 31 - protein, 6 aa, Homo sapiens
ARGFDY

SEQ ID NO: 32 - protein, 13 aa, Homo sapiens
ARGYSSGWYYFDY

SEQ ID NO: 33 - protein, 12 aa, Homo sapiens
ARDSSSWYAFDI

SEQ ID NO: 34 - protein, 13 aa, Homo sapiens
ARGYSSSWYAFDI

SEQ ID NO: 35 - protein, 16 aa, Homo sapiens
ARDSSSWYYYYYGMDV

SEQ ID NO: 36 - protein, 11 aa, Homo sapiens
ARGYSSGWYDY

SEQ ID NO: 37 - protein, 12 aa, Homo sapiens
ARGGSSWYYFDY

SEQ ID NO: 38 - protein, 9 aa, Homo sapiens
ARGGYYFDY

SEQ ID NO: 39 - protein, 13 aa, Homo sapiens
ARGDSSSWYYFDY

SEQ ID NO: 40 - protein, 14 aa, Homo sapiens
ARGGYSSSWYYFDY

SEQ ID NO: 41 - protein, 12 aa, Homo sapiens
ARVYSSSWYFDY

SEQ ID NO: 42 - protein, 11 aa, Homo sapiens
ARDRGSYYFDY

SEQ ID NO: 43 - protein, 12 aa, Homo sapiens
ARVSSSWYYFDY

SEQ ID NO: 44 - protein, 15 aa, Homo sapiens
ARDSGSYYYYYGMDV

SEQ ID NO: 45 - protein, 10 aa, Homo sapiens
ARDGGSYFDY

SEQ ID NO: 46 - protein, 15 aa, Homo sapiens
ARYYYDSSGYYYFDY

SEQ ID NO: 47 - protein, 12 aa, Homo sapiens
ARGSSGWYYFDY

SEQ ID NO: 48 - protein, 15 aa, Homo sapiens
ARGYYDSSGYYYFDY

SEQ ID NO: 49 - protein, 9 aa, Homo sapiens
ARGDYYFDY

SEQ ID NO: 50 - protein, 10 aa, Homo sapiens
ARGSSSWYDY

SEQ ID NO: 51 - protein, 11 aa, Homo sapiens
ARDRSGSYFDY

SEQ ID NO: 52 - protein, 13 aa, Homo sapiens
ARVYSSGWYYFDY

SEQ ID NO: 53 - protein, 5 aa, Homo sapiens
TTLDY

SEQ ID NO: 54 - protein, 12 aa, Homo sapiens
ARDSGSWYYFDY

SEQ ID NO: 55 - protein, 12 aa, Homo sapiens
ARDCSSTSCYDY

SEQ ID NO: 56 - protein, 6 aa, Homo sapiens
ARDFDY

SEQ ID NO: 57 - protein, 5 aa, Homo sapiens
ARDDY

SEQ ID NO: 58 - protein, 8 aa, Homo sapiens
ARDDAFDI

SEQ ID NO: 59 - protein, 12 aa, Homo sapiens
AKDSSSWYYFDY

SEQ ID NO: 60 - protein, 13 aa, Homo sapiens
ARDCSGGSCYFDY

SEQ ID NO: 61 - protein, 11 aa, Homo sapiens
ARDSSSYYFDY

SEQ ID NO: 62 - protein, 12 aa, Homo sapiens
ARGSSSWYYFDY

SEQ ID NO: 63 - protein, 11 aa, Homo sapiens
ARYSSSWYFDY

SEQ ID NO: 64 - protein, 11 aa, Homo sapiens
ARGYSSSWFDY

SEQ ID NO: 65 - protein, 14 aa, Homo sapiens
ARGYCSGGSCYFDY

SEQ ID NO: 66 - protein, 11 aa, Homo sapiens
AKDSSSWYFDY

SEQ ID NO: 67 - protein, 12 aa, Homo sapiens
ARDCSSTSCFDY

SEQ ID NO: 68 - protein, 11 aa, Homo sapiens
ARGSGSYYFDY

SEQ ID NO: 69 - protein, 5 aa, Homo sapiens
AREDY

SEQ ID NO: 70 - protein, 6 aa, Homo sapiens
ARDLGY

SEQ ID NO: 71 - protein, 15 aa, Homo sapiens
ARDYYDSSGYYYFDY

SEQ ID NO: 72 - protein, 5 aa, Homo sapiens
ARVDY

SEQ ID NO: 73 - protein, 13 aa, Homo sapiens
ARGYCSSTSCYDY

SEQ ID NO: 74 - protein, 13 aa, Homo sapiens
ARSYSSSWYYFDY

SEQ ID NO: 75 - protein, 11 aa, Homo sapiens
ARGSSSWYFDY

SEQ ID NO: 76 - protein, 5 aa, Homo sapiens
TTVDY

SEQ ID NO: 77 - protein, 12 aa, Homo sapiens
ARDSGSYYYFDY

SEQ ID NO: 78 - protein, 10 aa, Homo sapiens
ARDRGSYFDY

SEQ ID NO: 79 - protein, 8 aa, Homo sapiens
ARGDYFDY

SEQ ID NO: 80 - protein, 14 aa, Homo sapiens
ARGYCSSTSCYFDY

SEQ ID NO: 81 - protein, 9 aa, Homo sapiens
ARDRDAFDI

SEQ ID NO: 82 - protein, 12 aa, Homo sapiens
ARAYSSSWYFDY

SEQ ID NO: 83 - protein, 14 aa, Homo sapiens
ARGGYSSSWYWFDP

SEQ ID NO: 84 - protein, 6 aa, Homo sapiens
ARDYDY

SEQ ID NO: 85 - protein, 9 aa, Homo sapiens
ARDLDAFDI

SEQ ID NO: 86 - protein, 5 aa, Homo sapiens
AREGY

SEQ ID NO: 87 - protein, 12 aa, Homo sapiens
ARGSGSYYYFDY

SEQ ID NO: 88 - protein, 15 aa, Homo sapiens
ARGSGSYYYYYGMDV

SEQ ID NO: 89 - protein, 6 aa, Homo sapiens
ARELDY

SEQ ID NO: 90 - protein, 9 aa, Homo sapiens
ARGGDYFDY

SEQ ID NO: 91 - protein, 12 aa, Homo sapiens
ARGYSSSYYFDY

SEQ ID NO: 92 - protein, 9 aa, Homo sapiens
ARGVGATDY

SEQ ID NO: 93 - protein, 8 aa, Homo sapiens
ARDGAFDI

SEQ ID NO: 94 - protein, 14 aa, Homo sapiens
ARRGYSSSWYYFDY

SEQ ID NO: 95 - protein, 6 aa, Homo sapiens
ARGADY

SEQ ID NO: 96 - protein, 14 aa, Homo sapiens
ARGYSSSWYNWFDP

SEQ ID NO: 97 - protein, 15 aa, Homo sapiens
ARDSSSWYYYYGMDV

SEQ ID NO: 98 - protein, 10 aa, Homo sapiens
ARVYSSSFDY

SEQ ID NO: 99 - protein, 9 aa, Homo sapiens
QQSYSTPYT

SEQ ID NO: 100 - protein, 9 aa, Homo sapiens
QQYDNLPLT

SEQ ID NO: 101 - protein, 9 aa, Homo sapiens
QQSYSTPRT

SEQ ID NO: 102 - protein, 9 aa, Homo sapiens
QQYGSSPRT

SEQ ID NO: 103 - protein, 9 aa, Homo sapiens
QQYGSSPYT

SEQ ID NO: 104 - protein, 9 aa, Homo sapiens
QQSYSTPWT

SEQ ID NO: 105 - protein, 9 aa, Homo sapiens
QQYGSSPWT

SEQ ID NO: 106 - protein, 9 aa, Homo sapiens
QQYDNLPYT

SEQ ID NO: 107 - protein, 9 aa, Homo sapiens
QQSYSTPFT

SEQ ID NO: 108 - protein, 9 aa, Homo sapiens
QQYGSSPLT

SEQ ID NO: 109 - protein, 9 aa, Homo sapiens
QQSYSTPLT

SEQ ID NO: 110 - protein, 9 aa, Homo sapiens
LQHNSYPWT

SEQ ID NO: 111 - protein, 8 aa, Homo sapiens
QQYGSSPT

SEQ ID NO: 112 - protein, 9 aa, Homo sapiens
QQSYSTPPT

SEQ ID NO: 113 - protein, 10 aa, Homo sapiens [sequence omitted]

SEQ ID NO: 114 - protein, 10 aa, Homo sapiens
QQYGSSPPYT

SEQ ID NO: 115 - protein, 10 aa, Homo sapiens
QQYNNWPPYT

SEQ ID NO: 116 - protein, 9 aa, Homo sapiens
QQYYSYPRT

SEQ ID NO: 117 - protein, 9 aa, Homo sapiens
QQYNNWPRT

SEQ ID NO: 118 - protein, 9 aa, Homo sapiens
QQSYSTPIT

SEQ ID NO: 119 - protein, 9 aa, Homo sapiens
LQHNSYPYT

SEQ ID NO: 120 - protein, 9 aa, Homo sapiens
QQYDNLPIT

SEQ ID NO: 121 - protein, 9 aa, Homo sapiens
QQYGSSPFT

SEQ ID NO: 122 - protein, 9 aa, Homo sapiens
QQYGSSPIT

SEQ ID NO: 123 - protein, 10 aa, Homo sapiens
QQYNNWPPLT

SEQ ID NO: 124 - protein, 9 aa, Homo sapiens
QQYDNLPFT

SEQ ID NO: 125 - protein, 9 aa, Homo sapiens
QQRSNWPLT

SEQ ID NO: 126 - protein, 9 aa, Homo sapiens
LQHNSYPRT

SEQ ID NO: 127 - protein, 8 aa, Homo sapiens
QQYNSYST

SEQ ID NO: 128 - protein, 8 aa, Homo sapiens
QQYNSYWT

SEQ ID NO: 129 - protein, 9 aa, Homo sapiens
QQYYSTPYT

SEQ ID NO: 130 - protein, 10 aa, Homo sapiens
QQYGSSPPWT

SEQ ID NO: 131 - protein, 9 aa, Homo sapiens
QQYNSYPYT

SEQ ID NO: 132 - protein, 9 aa, Homo sapiens
QQYNNWPLT

SEQ ID NO: 133 - protein, 9 aa, Homo sapiens
QQYYSYPYT

SEQ ID NO: 134 - protein, 9 aa, Homo sapiens
QQYNSYSWT

SEQ ID NO: 135 - protein, 9 aa, Homo sapiens
QQYNSYPLT

SEQ ID NO: 136 - protein, 9 aa, Homo sapiens
QQYYSTPLT

SEQ ID NO: 137 - protein, 10 aa, Homo sapiens
QQRSNWPPIT

SEQ ID NO: 138 - protein, 9 aa, Homo sapiens
QQYNNWPYT

SEQ ID NO: 139 - protein, 9 aa, Homo sapiens
QQYYSYPWT

SEQ ID NO: 140 - protein, 9 aa, Homo sapiens
QQYNSYSRT

SEQ ID NO: 141 - protein, 9 aa, Homo sapiens
LQHNSYPLT

SEQ ID NO: 142 - protein, 10 aa, Homo sapiens
QQRSNWPPYT

SEQ ID NO: 143 - protein, 9 aa, Homo sapiens
QQYNSYSYT

SEQ ID NO: 144 - protein, 9 aa, Homo sapiens
QQYYSYPLT

SEQ ID NO: 145 - protein, 10 aa, Homo sapiens
QQRSNWPPLT

SEQ ID NO: 146 - protein, 8 aa, Homo sapiens
QQYGSSRT

SEQ ID NO: 147 - protein, 9 aa, Homo sapiens
QQYGSSPPT

SEQ ID NO: 148 - protein, 9 aa, Homo sapiens
QQYGSSPQT

SEQ ID NO: 149 - protein, 10 aa, Homo sapiens
QQYGSSPPIT

SEQ ID NO: 150 - protein, 9 aa, Homo sapiens
QQYNSYPWT

SEQ ID NO: 151 - protein, 9 aa, Homo sapiens
QQYYSTPWT

SEQ ID NO: 152 - protein, 9 aa, Homo sapiens
QQRSNWPPT

SEQ ID NO: 153 - protein, 9 aa, Homo sapiens
QQLNSYPLT

SEQ ID NO: 154 - protein, 9 aa, Homo sapiens
QQYYSYPFT

SEQ ID NO: 155 - protein, 9 aa, Homo sapiens
MQALQTPYT

SEQ ID NO: 156 - protein, 9 aa, Homo sapiens
QQYDNLPPT

SEQ ID NO: 157 - protein, 10 aa, Homo sapiens
QQYNNWPPIT

SEQ ID NO: 158 - protein, 10 aa, Homo sapiens
QQSYSTPPYT

SEQ ID NO: 159 - protein, 10 aa, Homo sapiens
QQYGSSPMYT

SEQ ID NO: 160 - protein, 8 aa, Homo sapiens
QQYGSSLT

SEQ ID NO: 161 - protein, 10 aa, Homo sapiens
QQYGSSPPLT

SEQ ID NO: 162 - protein, 9 aa, Homo sapiens
QQSYSTPHT

SEQ ID NO: 163 - protein, 9 aa, Homo sapiens
QQRSNWPIT

SEQ ID NO: 164 - protein, 9 aa, Homo sapiens
QQYGSSPGT

SEQ ID NO: 165 - protein, 10 aa, Homo sapiens
QQRSNWPPWT

SEQ ID NO: 166 - protein, 9 aa, Homo sapiens
QQYGSSLWT

SEQ ID NO: 167 - protein, 9 aa, Homo sapiens
QQRSNWPRT

SEQ ID NO: 168 - protein, 9 aa, Homo sapiens
QQYDNLPRT

SEQ ID NO: 169 - protein, 8 aa, Homo sapiens
QQSYSTPT

SEQ ID NO: 170 - protein, 10 aa, Homo sapiens
QQYDNLPPLT

SEQ ID NO: 171 - protein, 9 aa, Homo sapiens
QQSYSTPQT

SEQ ID NO: 172 - protein, 9 aa, Homo sapiens
MQALQTPLT

SEQ ID NO: 173 - protein, 9 aa, Homo sapiens
QQYGSSLYT

SEQ ID NO: 174 - protein, 8 aa, Homo sapiens
QQRSNWPT

SEQ ID NO: 175 - protein, 8 aa, Homo sapiens
QQYGSSYT

SEQ ID NO: 176 - protein, 10 aa, Homo sapiens
QQYDNLPPYT

SEQ ID NO: 177 - protein, 9 aa, Homo sapiens
QQYYSTPRT

SEQ ID NO: 178 - protein, 9 aa, Homo sapiens
QQSYSTPYS

SEQ ID NO: 179 - protein, 9 aa, Homo sapiens
QQSYSTPCS

SEQ ID NO: 180 - protein, 10 aa, Homo sapiens
QQSYSTPPWT

SEQ ID NO: 181 - protein, 9 aa, Homo sapiens
QQANSFPLT

SEQ ID NO: 182 - protein, 9 aa, Homo sapiens
QQLNSYPFT

SEQ ID NO: 183 - protein, 9 aa, Homo sapiens
QQLNSYPYT

SEQ ID NO: 184 - protein, 9 aa, Homo sapiens
QQYYSFPYT

SEQ ID NO: 185 - protein, 9 aa, Homo sapiens
MQALQTPWT

SEQ ID NO: 186 - protein, 8 aa, Homo sapiens
QQYGSSFT

SEQ ID NO: 187 - protein, 9 aa, Homo sapiens
QQLNSYPRT

SEQ ID NO: 188 - protein, 9 aa, Homo sapiens
QQSYSTLWT

SEQ ID NO: 189 - protein, 9 aa, Homo sapiens
LQHNSYPPT

SEQ ID NO: 190 - protein, 10 aa, Homo sapiens
QQRSNWPPFT

SEQ ID NO: 191 - protein, 8 aa, Homo sapiens
QQRSNWYT

SEQ ID NO: 192 - protein, 9 aa, Homo sapiens
QQYNSYPIT

SEQ ID NO: 193 - protein, 9 aa, Homo sapiens
QQYNSYPFT

SEQ ID NO: 194 - protein, 9 aa, Homo sapiens
QQYYSTPPT

SEQ ID NO: 195 - protein, 9 aa, Homo sapiens
QQYYSTPFT

SEQ ID NO: 196 - protein, 9 aa, Homo sapiens
MQALQTPRT

SEQ ID NO: 197 - protein, 11 aa, Homo sapiens
GTWDSSLSAVV

SEQ ID NO: 198 - protein, 10 aa, Homo sapiens
SSYTSSSTLV

SEQ ID NO: 199 - protein, 11 aa, Homo sapiens
GTWDSSLSAGV

SEQ ID NO: 200 - protein, 10 aa, Homo sapiens
SSYTSSSTVV

SEQ ID NO: 201 - protein, 11 aa, Homo sapiens
QSYDSSLSGSV

SEQ ID NO: 202 - protein, 10 aa, Homo sapiens
SSYTSSSTWV

SEQ ID NO: 203 - protein, 11 aa, Homo sapiens
SSYTSSSTLVV

SEQ ID NO: 204 - protein, 11 aa, Homo sapiens
QVWDSSSDHVV

SEQ ID NO: 205 - protein, 11 aa, Homo sapiens
NSRDSSGNHLV

SEQ ID NO: 206 - protein, 11 aa, Homo sapiens
QSADSSGTYVV

SEQ ID NO: 207 - protein, 10 aa, Homo sapiens
SSYTSSSTYV

SEQ ID NO: 208 - protein, 10 aa, Homo sapiens
SSYAGSNNLV

SEQ ID NO: 209 - protein, 11 aa, Homo sapiens
NSRDSSGNHVV

SEQ ID NO: 210 - protein, 11 aa, Homo sapiens
QSYDSSLSGWV

SEQ ID NO: 211 - protein, 11 aa, Homo sapiens
AAWDDSLNGPV

SEQ ID NO: 212 - protein, 11 aa, Homo sapiens
QSYDSSLSGYV

SEQ ID NO: 213 - protein, 11 aa, Homo sapiens
NSRDSSGNHWV

SEQ ID NO: 214 - protein, 10 aa, Homo sapiens
CSYAGSSTLV

SEQ ID NO: 215 - protein, 11 aa, Homo sapiens
GTWDSSLSAWV

SEQ ID NO: 216 - protein, 11 aa, Homo sapiens
AAWDDSLNGWV

SEQ ID NO: 217 - protein, 11 aa, Homo sapiens
QVWDSSSDHPV

SEQ ID NO: 218 - protein, 10 aa, Homo sapiens
SSYTSSSTRV

SEQ ID NO: 219 - protein, 11 aa, Homo sapiens
QVWDSSSDHWV

SEQ ID NO: 220 - protein, 11 aa, Homo sapiens
QSYDSSLSGVV

SEQ ID NO: 221 - protein, 11 aa, Homo sapiens
GTWDSSLSAYV

SEQ ID NO: 222 - protein, 9 aa, Homo sapiens
QAWDSSTVV

SEQ ID NO: 223 - protein, 10 aa, Homo sapiens
CSYAGSSTWV

SEQ ID NO: 224 - protein, 10 aa, Homo sapiens
CSYAGSYTLV

SEQ ID NO: 225 - protein, 10 aa, Homo sapiens
CSYAGSSTYV

SEQ ID NO: 226 - protein, 10 aa, Homo sapiens
CSYAGSYTYV

SEQ ID NO: 227 - protein, 9 aa, Homo sapiens
SSYTSSSTV

SEQ ID NO: 228 - protein, 10 aa, Homo sapiens
SSYAGSNNVV

SEQ ID NO: 229 - protein, 10 aa, Homo sapiens
CSYAGSYTWV

SEQ ID NO: 230 - protein, 11 aa, Homo sapiens
AAWDDSLNGVV

SEQ ID NO: 231 - protein, 11 aa, Homo sapiens
QSADSSGTYWV

SEQ ID NO: 232 - protein, 10 aa, Homo sapiens
CSYAGSSTVV

SEQ ID NO: 233 - protein, 10 aa, Homo sapiens
QSADSSGTWV

SEQ ID NO: 234 - protein, 11 aa, Homo sapiens
QVWDSSSDHYV

SEQ ID NO: 235 - protein, 10 aa, Homo sapiens
GTWDSSLSAV

SEQ ID NO: 236 - protein, 11 aa, Homo sapiens
SSYTSSSTLYV

SEQ ID NO: 237 - protein, 10 aa, Homo sapiens
QSYDSSLSVV

SEQ ID NO: 238 - protein, 11 aa, Homo sapiens
CSYAGSSTFVV

SEQ ID NO: 239 - protein, 9 aa, Homo sapiens
MIWHSSAWV

SEQ ID NO: 240 - protein, 10 aa, Homo sapiens
SSYAGSNNYV

SEQ ID NO: 241 - protein, 11 aa, Homo sapiens
NSRDSSGNHYV

SEQ ID NO: 242 - protein, 11 aa, Homo sapiens
AAWDDSLSGWV

SEQ ID NO: 243 - protein, 9 aa, Homo sapiens
SSYTSSSVV

SEQ ID NO: 244 - protein, 11 aa, Homo sapiens
AAWDDSLNGYV

SEQ ID NO: 245 - protein, 11 aa, Homo sapiens
CSYAGSSTYVV

SEQ ID NO: 246 - protein, 11 aa, Homo sapiens
NSRDSSGNHRV

SEQ ID NO: 247 - protein, 11 aa, Homo sapiens
SSYTSSSTPVV

SEQ ID NO: 248 - protein, 11 aa, Homo sapiens
SSYTSSSTYVV

SEQ ID NO: 249 - protein, 11 aa, Homo sapiens
SSYTSSSTHVV

SEQ ID NO: 250 - protein, 11 aa, Homo sapiens
GTWDSSLSVVV

SEQ ID NO: 251 - protein, 12 aa, Homo sapiens
QSYDSSLSGSVV

SEQ ID NO: 252 - protein, 10 aa, Homo sapiens
VLYMGSGISV

SEQ ID NO: 253 - protein, 11 aa, Homo sapiens
SSYTSSSTLGV

SEQ ID NO: 254 - protein, 11 aa, Homo sapiens
SSYAGSNNFVV

SEQ ID NO: 255 - protein, 12 aa, Homo sapiens
QSYDSSLSGYVV

SEQ ID NO: 256 - protein, 11 aa, Homo sapiens
SAWDSSLSAWV

SEQ ID NO: 257 - protein, 10 aa, Homo sapiens
CSYAGSYTVV

SEQ ID NO: 258 - protein, 11 aa, Homo sapiens
QVWDSSSDHRV

SEQ ID NO: 259 - protein, 11 aa, Homo sapiens
AAWDDSLSGPV

SEQ ID NO: 260 - protein, 12 aa, Homo sapiens
SSYTSSSTLDVV

SEQ ID NO: 261 - protein, 11 aa, Homo sapiens
QSADSSGTYRV

SEQ ID NO: 262 - protein, 9 aa, Homo sapiens
SSYTSSSTL

SEQ ID NO: 263 - protein, 11 aa, Homo sapiens
AAWDDSLNGRV

SEQ ID NO: 264 - protein, 9 aa, Homo sapiens
LLSYSGARV

SEQ ID NO: 265 - protein, 10 aa, Homo sapiens
VLYMGSGIWV

SEQ ID NO: 266 - protein, 11 aa, Homo sapiens
SSYTSSSTLEV

SEQ ID NO: 267 - protein, 11 aa, Homo sapiens
AAWDDSLSGRV

SEQ ID NO: 268 - protein, 10 aa, Homo sapiens
QSADSSGTYV

SEQ ID NO: 269 - protein, 9 aa, Homo sapiens
QAWDSSTAV

SEQ ID NO: 270 - protein, 10 aa, Homo sapiens
QSADSSGTVV

SEQ ID NO: 271 - protein, 10 aa, Homo sapiens
CSYAGSSTFV

SEQ ID NO: 272 - protein, 11 aa, Homo sapiens
CSYAGSYTFVV

SEQ ID NO: 273 - protein, 11 aa, Homo sapiens
AAWDDSLSGVV

SEQ ID NO: 274 - protein, 10 aa, Homo sapiens
GTWDSSLSVV

SEQ ID NO: 275 - protein, 9 aa, Homo sapiens
QSYDSSNVV

SEQ ID NO: 276 - protein, 10 aa, Homo sapiens
CSYAGSYTFV

SEQ ID NO: 277 - protein, 9 aa, Homo sapiens
QTWGTGIQV

SEQ ID NO: 278 - protein, 10 aa, Homo sapiens
SSYAGSNNFV

SEQ ID NO: 279 - protein, 11 aa, Homo sapiens
QSYDSSLSDVV

SEQ ID NO: 280 - protein, 10 aa, Homo sapiens
SSYAGSNNWV

SEQ ID NO: 281 - protein, 11 aa, Homo sapiens
GTWDSSLSAEV

SEQ ID NO: 282 - protein, 12 aa, Homo sapiens
QSYDSSLSGSRV

SEQ ID NO: 283 - protein, 9 aa, Homo sapiens
MIWHSSAVV

SEQ ID NO: 284 - protein, 8 aa, Homo sapiens
CSYAGSYV

SEQ ID NO: 285 - protein, 11 aa, Homo sapiens
GTWDSSLSVWV

SEQ ID NO: 286 - protein, 9 aa, Homo sapiens
ETWDSNTRV

SEQ ID NO: 287 - protein, 12 aa, Homo sapiens
GTWDSSLSAGGV

SEQ ID NO: 288 - protein, 11 aa, Homo sapiens
GTWDSSLSDVV

SEQ ID NO: 289 - protein, 9 aa, Homo sapiens
QSYDSSNQV

SEQ ID NO: 290 - protein, 9 aa, Homo sapiens
CSYAGSYTV

SEQ ID NO: 291 - protein, 11 aa, Homo sapiens
SSYTSSSTPYV

SEQ ID NO: 292 - protein, 10 aa, Homo sapiens
SSYTSSSTPV

SEQ ID NO: 293 - protein, 12 aa, Homo sapiens
GTWDSSLSAGRV

SEQ ID NO: 294 - protein, 12 aa, Homo sapiens
NSRDSSGNHLVV

SEQ ID NO: 295 - protein, 13 aa, Homo sapiens
VVSDRGSTLGRLY

SEQ ID NO: 296 - protein, 10 aa, Homo sapiens
AVNTGGFKTI

SEQ ID NO: 297 - protein, 8 aa, Homo sapiens
AVNDYKLS

SEQ ID NO: 298 - protein, 10 aa, Homo sapiens
AVNQAGTALI

SEQ ID NO: 299 - protein, 10 aa, Homo sapiens
AVNTGFQKLV

SEQ ID NO: 300 - protein, 11 aa, Homo sapiens
AVNSGGYQKVT

SEQ ID NO: 301 - protein, 10 aa, Homo sapiens
AVNTNAGKST

SEQ ID NO: 302 - protein, 10 aa, Homo sapiens
AATDSWGKLQ

SEQ ID NO: 303 - protein, 11 aa, Homo sapiens
AVRDTGGFKTI

SEQ ID NO: 304 - protein, 10 aa, Homo sapiens
AVMDSSYKLI

SEQ ID NO: 305 - protein, 10 aa, Homo sapiens
AVDTGRRALT

SEQ ID NO: 306 - protein, 9 aa, Homo sapiens
AVNRDDKII

SEQ ID NO: 307 - protein, 10 aa, Homo sapiens
AVNSGYSTLT

SEQ ID NO: 308 - protein, 11 aa, Homo sapiens
AVRDTGRRALT

SEQ ID NO: 309 - protein, 9 aa, Homo sapiens
AVNTGNQFY

SEQ ID NO: 310 - protein, 8 aa, Homo sapiens
AVTGNQFY

SEQ ID NO: 311 - protein, 10 aa, Homo sapiens
VVNTNAGKST

SEQ ID NO: 312 - protein, 10 aa, Homo sapiens
AVDNYGQNFV

SEQ ID NO: 313 - protein, 10 aa, Homo sapiens
AVKAAGNKLT

SEQ ID NO: 314 - protein, 9 aa, Homo sapiens
AVYNFNKFY

SEQ ID NO: 315 - protein, 9 aa, Homo sapiens
AVDSNYQLI

SEQ ID NO: 316 - protein, 9 aa, Homo sapiens
AVSNDYKLS

SEQ ID NO: 317 - protein, 10 aa, Homo sapiens
VVNQAGTALI

SEQ ID NO: 318 - protein, 9 aa, Homo sapiens
AASNDYKLS

SEQ ID NO: 319 - protein, 11 aa, Homo sapiens
AVRDNYGQNFV

SEQ ID NO: 320 - protein, 10 aa, Homo sapiens
AVSGSARQLT

SEQ ID NO: 321 - protein, 12 aa, Homo sapiens
AVNYGGSQGNLI

SEQ ID NO: 322 - protein, 10 aa, Homo sapiens
AVNNAGNMLT

SEQ ID NO: 323 - protein, 10 aa, Homo sapiens
AASGGSYIPT

SEQ ID NO: 324 - protein, 10 aa, Homo sapiens
AVNSGNTPLV

SEQ ID NO: 325 - protein, 11 aa, Homo sapiens
AVRNTGGFKTI

SEQ ID NO: 326 - protein, 10 aa, Homo sapiens
AVSGGSYIPT

SEQ ID NO: 327 - protein, 11 aa, Homo sapiens
AVFSGGYNKLI

SEQ ID NO: 328 - protein, 10 aa, Homo sapiens
AVSGGYQKVT

SEQ ID NO: 329 - protein, 10 aa, Homo sapiens
AASTGGFKTI

SEQ ID NO: 330 - protein, 11 aa, Homo sapiens
AVRDQAGTALI

SEQ ID NO: 331 - protein, 11 aa, Homo sapiens
AASKGGSYIPT

SEQ ID NO: 332 - protein, 10 aa, Homo sapiens
ALNTGGFKTI

SEQ ID NO: 333 - protein, 10 aa, Homo sapiens
AVMDSNYQLI

SEQ ID NO: 334 - protein, 12 aa, Homo sapiens
AVNSGGSNYKLT

SEQ ID NO: 335 - protein, 8 aa, Homo sapiens
AVNTDKLI

SEQ ID NO: 336 - protein, 8 aa, Homo sapiens
AVRDDKII

SEQ ID NO: 337 - protein, 9 aa, Homo sapiens
ALYNFNKFY

SEQ ID NO: 338 - protein, 9 aa, Homo sapiens
AVSGNTPLV

SEQ ID NO: 339 - protein, 11 aa, Homo sapiens
AASTSGTYKYI

SEQ ID NO: 340 - protein, 10 aa, Homo sapiens
AVYTGGFKTI

SEQ ID NO: 341 - protein, 11 aa, Homo sapiens
AVASGGSYIPT

SEQ ID NO: 342 - protein, 10 aa, Homo sapiens
AVDTGGFKTI

SEQ ID NO: 343 - protein, 10 aa, Homo sapiens
AVTSGTYKYI

SEQ ID NO: 344 - protein, 11 aa, Homo sapiens
AVSDTGGFKTI

SEQ ID NO: 345 - protein, 10 aa, Homo sapiens
AVYSSASKII

SEQ ID NO: 346 - protein, 11 aa, Homo sapiens
AVIKAAGNKLT

SEQ ID NO: 347 - protein, 8 aa, Homo sapiens
VVNDYKLS

SEQ ID NO: 348 - protein, 11 aa, Homo sapiens
AVLNQAGTALI

SEQ ID NO: 349 - protein, 8 aa, Homo sapiens
AANDYKLS

SEQ ID NO: 350 - protein, 10 aa, Homo sapiens
AVRGGSYIPT

SEQ ID NO: 351 - protein, 11 aa, Homo sapiens
LVGDTGRRALT

SEQ ID NO: 352 - protein, 9 aa, Homo sapiens
AVRDDYKLS

SEQ ID NO: 353 - protein, 8 aa, Homo sapiens
AVDDYKLS

SEQ ID NO: 354 - protein, 8 aa, Homo sapiens
ALNDYKLS

SEQ ID NO: 355 - protein, 10 aa, Homo sapiens
AVRSNDYKLS

SEQ ID NO: 356 - protein, 11 aa, Homo sapiens
AVRSGGSYIPT

SEQ ID NO: 357 - protein, 10 aa, Homo sapiens
AVRDSNYQLI

SEQ ID NO: 358 - protein, 10 aa, Homo sapiens
AVGGSQGNLI

SEQ ID NO: 359 - protein, 10 aa, Homo sapiens
AVTGGGNKLT

SEQ ID NO: 360 - protein, 10 aa, Homo sapiens
VVNTGGFKTI

SEQ ID NO: 361 - protein, 11 aa, Homo sapiens
AVPNQAGTALI

SEQ ID NO: 362 - protein, 10 aa, Homo sapiens
VVNTGFQKLV

SEQ ID NO: 363 - protein, 10 aa, Homo sapiens
AANTGGFKTI

SEQ ID NO: 364 - protein, 10 aa, Homo sapiens
AVNFGNEKLT

SEQ ID NO: 365 - protein, 11 aa, Homo sapiens
AVRMDSSYKLI

SEQ ID NO: 366 - protein, 10 aa, Homo sapiens
AVSSNDYKLS

SEQ ID NO: 367 - protein, 11 aa, Homo sapiens
AVSNQAGTALI

SEQ ID NO: 368 - protein, 9 aa, Homo sapiens
AVTGGFKTI

SEQ ID NO: 369 - protein, 11 aa, Homo sapiens
AVEDTGGFKTI

SEQ ID NO: 370 - protein, 11 aa, Homo sapiens
AVGSSNTGKLI

SEQ ID NO: 371 - protein, 11 aa, Homo sapiens
AASNQAGTALI

SEQ ID NO: 372 - protein, 10 aa, Homo sapiens
AANFGNEKLT

SEQ ID NO: 373 - protein, 12 aa, Homo sapiens
AVHGSSNTGKLI

SEQ ID NO: 374 - protein, 10 aa, Homo sapiens
AANQAGTALI

SEQ ID NO: 375 - protein, 11 aa, Homo sapiens
AVNAGNNRKLI

SEQ ID NO: 376 - protein, 10 aa, Homo sapiens
AVSGGYNKLI

SEQ ID NO: 377 - protein, 9 aa, Homo sapiens
AGGSQGNLI

SEQ ID NO: 378 - protein, 10 aa, Homo sapiens
AVGSNDYKLS

SEQ ID NO: 379 - protein, 9 aa, Homo sapiens
AVYNNNDMR

SEQ ID NO: 380 - protein, 11 aa, Homo sapiens
AVANQAGTALI

SEQ ID NO: 381 - protein, 12 aa, Homo sapiens
AVDRGSTLGRLY

SEQ ID NO: 382 - protein, 11 aa, Homo sapiens
AVTTDSWGKLQ

SEQ ID NO: 383 - protein, 10 aa, Homo sapiens
ALDTGRRALT

SEQ ID NO: 384 - protein, 11 aa, Homo sapiens
AVNSNSGYALN

SEQ ID NO: 385 - protein, 11 aa, Homo sapiens
AVSSGGYQKVT

SEQ ID NO: 386 - protein, 10 aa, Homo sapiens
AATSGTYKYI

SEQ ID NO: 387 - protein, 11 aa, Homo sapiens
AVNNQAGTALI

SEQ ID NO: 388 - protein, 11 aa, Homo sapiens
AVEDTGRRALT

SEQ ID NO: 389 - protein, 10 aa, Homo sapiens
AVRGSQGNLI

SEQ ID NO: 390 - protein, 10 aa, Homo sapiens
AVNNYGQNFV

SEQ ID NO: 391 - protein, 9 aa, Homo sapiens
AVNNFNKFY

SEQ ID NO: 392 - protein, 12 aa, Homo sapiens
AVRDSGGYQKVT

SEQ ID NO: 393 - protein, 11 aa, Homo sapiens
ASSLGQNTEAF

SEQ ID NO: 394 - protein, 12 aa, Homo sapiens
ASSLAGGTDTQY

SEQ ID NO: 395 - protein, 9 aa, Homo sapiens
ASSLGYEQY

SEQ ID NO: 396 - protein, 10 aa, Homo sapiens
ASSLQNTEAF

SEQ ID NO: 397 - protein, 9 aa, Homo sapiens
ASSLADTQY

SEQ ID NO: 398 - protein, 10 aa, Homo sapiens
ASSLGNTEAF

SEQ ID NO: 399 - protein, 10 aa, Homo sapiens
ASSLGGTEAF

SEQ ID NO: 400 - protein, 12 aa, Homo sapiens
ASSLGTGGYEQY

SEQ ID NO: 401 - protein, 11 aa, Homo sapiens
ASSLQGNQPQH

SEQ ID NO: 402 - protein, 9 aa, Homo sapiens
ASSFTDTQY

SEQ ID NO: 403 - protein, 9 aa, Homo sapiens
ASSLTDTQY

SEQ ID NO: 404 - protein, 8 aa, Homo sapiens
ASSQETQY

SEQ ID NO: 405 - protein, 9 aa, Homo sapiens
ASSLSYEQY

SEQ ID NO: 406 - protein, 9 aa, Homo sapiens
ASSLEETQY

SEQ ID NO: 407 - protein, 10 aa, Homo sapiens
ASSLGGYEQY

SEQ ID NO: 408 - protein, 12 aa, Homo sapiens
ASSLAGGPDTQY

SEQ ID NO: 409 - protein, 11 aa, Homo sapiens
ASSLTGNTEAF

SEQ ID NO: 410 - protein, 11 aa, Homo sapiens
ASSLGGTDTQY

SEQ ID NO: 411 - protein, 9 aa, Homo sapiens
ASSLNTEAF

SEQ ID NO: 412 - protein, 9 aa, Homo sapiens
ASSSSYEQY

SEQ ID NO: 413 - protein, 10 aa, Homo sapiens
ASSLDTYEQY

SEQ ID NO: 414 - protein, 10 aa, Homo sapiens
ASSPSTDTQY

SEQ ID NO: 415 - protein, 11 aa, Homo sapiens
ASSLGQGYEQY

SEQ ID NO: 416 - protein, 11 aa, Homo sapiens
ASSLTGGYEQY

SEQ ID NO: 417 - protein, 10 aa, Homo sapiens
ASSLGTDTQY

SEQ ID NO: 418 - protein, 11 aa, Homo sapiens
ASSLAGTDTQY

SEQ ID NO: 419 - protein, 11 aa, Homo sapiens
ASSLDSSYEQY

SEQ ID NO: 420 - protein, 11 aa, Homo sapiens
ASSLDSNQPQH

SEQ ID NO: 421 - protein, 10 aa, Homo sapiens
ASSLSTDTQY

SEQ ID NO: 422 - protein, 10 aa, Homo sapiens
SARQGNQPQH

SEQ ID NO: 423 - protein, 10 aa, Homo sapiens
ASSLDSYEQY

SEQ ID NO: 424 - protein, 9 aa, Homo sapiens
ASSSTDTQY

SEQ ID NO: 425 - protein, 11 aa, Homo sapiens
ASSLGGNQPQH

SEQ ID NO: 426 - protein, 11 aa, Homo sapiens
ASSLDSTDTQY

SEQ ID NO: 427 - protein, 12 aa, Homo sapiens
ASSLTSGTDTQY

SEQ ID NO: 428 - protein, 9 aa, Homo sapiens
ASSLGTEAF

SEQ ID NO: 429 - protein, 9 aa, Homo sapiens
ASSYSYEQY

SEQ ID NO: 430 - protein, 9 aa, Homo sapiens
ASSQGYEQY

SEQ ID NO: 431 - protein, 11 aa, Homo sapiens
ASSLQGNTEAF

SEQ ID NO: 432 - protein, 11 aa, Homo sapiens
ASSLTGGTEAF

SEQ ID NO: 433 - protein, 9 aa, Homo sapiens
ASSLGYGYT

SEQ ID NO: 434 - protein, 11 aa, Homo sapiens
ASSLGGNTEAF

SEQ ID NO: 435 - protein, 10 aa, Homo sapiens
ASSLQGYEQY

SEQ ID NO: 436 - protein, 12 aa, Homo sapiens
ASSLGQGTDTQY

SEQ ID NO: 437 - protein, 9 aa, Homo sapiens
ASSLGETQY

SEQ ID NO: 438 - protein, 12 aa, Homo sapiens
ASSLTGGTDTQY

SEQ ID NO: 439 - protein, 13 aa, Homo sapiens
ASSLLAGGTDTQY

SEQ ID NO: 440 - protein, 12 aa, Homo sapiens
ASSLLAGTDTQY

SEQ ID NO: 441 - protein, 9 aa, Homo sapiens
ASSLAYEQY

SEQ ID NO: 442 - protein, 9 aa, Homo sapiens
ASSSQETQY

SEQ ID NO: 443 - protein, 9 aa, Homo sapiens
ASSPSYEQY

SEQ ID NO: 444 - protein, 9 aa, Homo sapiens
ASSLGGEQY

SEQ ID NO: 445 - protein, 11 aa, Homo sapiens
ASSLTSTDTQY

SEQ ID NO: 446 - protein, 9 aa, Homo sapiens
ASSLGDTQY

SEQ ID NO: 447 - protein, 10 aa, Homo sapiens
ASSLSSYEQY

SEQ ID NO: 448 - protein, 9 aa, Homo sapiens
ASSYTDTQY

SEQ ID NO: 449 - protein, 11 aa, Homo sapiens
ASSLASTDTQY

SEQ ID NO: 450 - protein, 11 aa, Homo sapiens
ASSLGQNYGYT

SEQ ID NO: 451 - protein, 10 aa, Homo sapiens
ASSYSTDTQY

SEQ ID NO: 452 - protein, 12 aa, Homo sapiens
ASSLAGGSYEQY

SEQ ID NO: 453 - protein, 9 aa, Homo sapiens
ASSRTDTQY

SEQ ID NO: 454 - protein, 9 aa, Homo sapiens
ASSYTYEQY

SEQ ID NO: 455 - protein, 10 aa, Homo sapiens
ASSLAGYEQY

SEQ ID NO: 456 - protein, 9 aa, Homo sapiens
ASSFYNEQF

SEQ ID NO: 457 - protein, 10 aa, Homo sapiens
ASSLTGYEQY

SEQ ID NO: 458 - protein, 10 aa, Homo sapiens
ASSRDTYEQY

SEQ ID NO: 459 - protein, 10 aa, Homo sapiens
ASRQGNQPQH

SEQ ID NO: 460 - protein, 12 aa, Homo sapiens
ASSLAGGQETQY

SEQ ID NO: 461 - protein, 10 aa, Homo sapiens
ASSLGSYEQY

SEQ ID NO: 462 - protein, 8 aa, Homo sapiens
ASSSYEQY

SEQ ID NO: 463 - protein, 11 aa, Homo sapiens
ASSLAGNTEAF

SEQ ID NO: 464 - protein, 9 aa, Homo sapiens
ASSPQETQY

SEQ ID NO: 465 - protein, 12 aa, Homo sapiens
SARLAGGTDTQY

SEQ ID NO: 466 - protein, 10 aa, Homo sapiens
ASSLTTDTQY

SEQ ID NO: 467 - protein, 11 aa, Homo sapiens
ASSLDRNTEAF

SEQ ID NO: 468 - protein, 11 aa, Homo sapiens
ASSPGQNTEAF

SEQ ID NO: 469 - protein, 10 aa, Homo sapiens
ASSYSNQPQH

SEQ ID NO: 470 - protein, 11 aa, Homo sapiens
ASSQDRGYEQY

SEQ ID NO: 471 - protein, 12 aa, Homo sapiens
ASSLGGSNQPQH

SEQ ID NO: 472 - protein, 11 aa, Homo sapiens
ASSLTGNQPQH

SEQ ID NO: 473 - protein, 11 aa, Homo sapiens
ASSLAGGTEAF

SEQ ID NO: 474 - protein, 10 aa, Homo sapiens
ASSLTGYGYT

SEQ ID NO: 475 - protein, 10 aa, Homo sapiens
ASSYSSYEQY

SEQ ID NO: 476 - protein, 11 aa, Homo sapiens
ASSGQGNQPQH

SEQ ID NO: 477 - protein, 10 aa, Homo sapiens
ASSLANYGYT

SEQ ID NO: 478 - protein, 11 aa, Homo sapiens
ASSLAGAYEQY

SEQ ID NO: 479 - protein, 9 aa, Homo sapiens
ASSLKETQY

SEQ ID NO: 480 - protein, 12 aa, Homo sapiens
ASSPGQGAYEQY

SEQ ID NO: 481 - protein, 12 aa, Homo sapiens
ASSLGGSTDTQY

SEQ ID NO: 482 - protein, 11 aa, Homo sapiens
ASSLDRDTEAF

SEQ ID NO: 483 - protein, 10 aa, Homo sapiens
ASSFSNQPQH

SEQ ID NO: 484 - protein, 11 aa, Homo sapiens
ASSRTGGYEQY

SEQ ID NO: 485 - protein, 10 aa, Homo sapiens
ASRDSNQPQH

SEQ ID NO: 486 - protein, 13 aa, Homo sapiens
ASSLLAGGYNEQF

SEQ ID NO: 487 - protein, 11 aa, Homo sapiens
ASSLQGAYEQY

SEQ ID NO: 488 - protein, 11 aa, Homo sapiens
ASSLGVNTEAF

SEQ ID NO: 489 - protein, 11 aa, Homo sapiens
ASSLGLNTEAF

SEQ ID NO: 490 - protein, 13 aa, Homo sapiens
ASSLLAGGPDTQY

SEQ ID NO: 491 - protein, 14 aa, Homo sapiens
ACDWGSSWDTRQMF

SEQ ID NO: 492 - protein, 12 aa, Homo sapiens
ACDTGGYTDKLI

SEQ ID NO: 493 - protein, 12 aa, Homo sapiens
ACDTGGYADKLI

SEQ ID NO: 494 - protein, 15 aa, Homo sapiens
ACDTGGYSWDTRQMF

SEQ ID NO: 495 - protein, 12 aa, Homo sapiens
ACDILGDTDKLI

SEQ ID NO: 496 - protein, 12 aa, Homo sapiens
ACDVLGDTDKLI

SEQ ID NO: 497 - protein, 14 aa, Homo sapiens
ACDWGSSWDTRQMS

SEQ ID NO: 498 - protein, 13 aa, Homo sapiens
ACDWGSSDTRQMF

SEQ ID NO: 499 - protein, 12 aa, Homo sapiens
ACDILGDTAQLF

SEQ ID NO: 500 - protein, 14 aa, Homo sapiens
ACDILGDTLTAQLF

SEQ ID NO: 501 - protein, 16 aa, Homo sapiens
ACDTAGGYSWDTRQMF

SEQ ID NO: 502 - protein, 12 aa, Homo sapiens
ACDVLGDTAQLF

SEQ ID NO: 503 - protein, 13 aa, Homo sapiens
ACDTGGYLTAQLF

SEQ ID NO: 504 - protein, 14 aa, Homo sapiens
ACGWGSSWDTRQMF

SEQ ID NO: 505 - protein, 15 aa, Homo sapiens
ACDILGDTWDTRQMF

SEQ ID NO: 506 - protein, 18 aa, Homo sapiens
ACDTVLGDTSSWDTRQMF

SEQ ID NO: 507 - protein, 14 aa, Homo sapiens
ACDWGGSWDTRQMF

SEQ ID NO: 508 - protein, 14 aa, Homo sapiens
ACDRGSSWDTRQMF

SEQ ID NO: 509 - protein, 14 aa, Homo sapiens
ACDWGSSWDTRQML

SEQ ID NO: 510 - protein, 14 aa, Homo sapiens
ACDWGSPWDTRQMF

SEQ ID NO: 511 - protein, 14 aa, Homo sapiens
ACDTGGYWDTRQMF

SEQ ID NO: 512 - protein, 12 aa, Homo sapiens
ACDTWGMTAQLF

SEQ ID NO: 513 - protein, 16 aa, Homo sapiens
ACDTVLGDTWDTRQMF

SEQ ID NO: 514 - protein, 11 aa, Homo sapiens
ACDTWDTRQMF

SEQ ID NO: 515 - protein, 15 aa, Homo sapiens
ACDTWGSSWDTRQMF

SEQ ID NO: 516 - protein, 12 aa, Homo sapiens
ACDTWGYTDKLI

SEQ ID NO: 517 - protein, 17 aa, Homo sapiens
ACDTVLGDSSWDTRQMF

SEQ ID NO: 518 - protein, 15 aa, Homo sapiens
ACDVLGDTWDTRQMF

SEQ ID NO: 519 - protein, 14 aa, Homo sapiens
ACDWGSSWGTRQMF

SEQ ID NO: 520 - protein, 14 aa, Homo sapiens
ACDWGSSWDTRQVF

SEQ ID NO: 521 - protein, 14 aa, Homo sapiens
ACDWGSSWDARQMF

SEQ ID NO: 522 - protein, 14 aa, Homo sapiens
ACDWGSSWDTRRMF

SEQ ID NO: 523 - protein, 11 aa, Homo sapiens
ACDTWGTAQLF

SEQ ID NO: 524 - protein, 12 aa, Homo sapiens
ACDTVGDTDKLI

SEQ ID NO: 525 - protein, 16 aa, Homo sapiens
ACDTGGYSSWDTRQMF

SEQ ID NO: 526 - protein, 14 aa, Homo sapiens
ACDTLGDTLTAQLF

SEQ ID NO: 527 - protein, 16 aa, Homo sapiens
ACDTGGYGSWDTRQMF

SEQ ID NO: 528 - protein, 14 aa, Homo sapiens
ACDWGSSRDTRQMF

SEQ ID NO: 529 - protein, 14 aa, Homo sapiens
ARDWGSSWDTRQMF

SEQ ID NO: 530 - protein, 14 aa, Homo sapiens
ACVWGSSWDTRQMF

SEQ ID NO: 531 - protein, 12 aa, Homo sapiens
ACDTGGLTAQLF

SEQ ID NO: 532 - protein, 14 aa, Homo sapiens
ACDTGGSWDTRQMF

SEQ ID NO: 533 - protein, 13 aa, Homo sapiens
ACDWGTWDTRQMF

SEQ ID NO: 534 - protein, 13 aa, Homo sapiens
ACDILGDLTAQLF

SEQ ID NO: 535 - protein, 16 aa, Homo sapiens
ACDTVLGDSWDTRQMF

SEQ ID NO: 536 - protein, 13 aa, Homo sapiens
ACDWGSSWDTQMF

SEQ ID NO: 537 - protein, 12 aa, Homo sapiens
ACDTGGYTDKPI

SEQ ID NO: 538 - protein, 14 aa, Homo sapiens
ACDWGSSWDTQQMF

SEQ ID NO: 539 - protein, 14 aa, Homo sapiens
ACDWGCSWDTRQMF

SEQ ID NO: 540 - protein, 15 aa, Homo sapiens
ACDYWGSSWDTRQMF

SEQ ID NO: 541 - protein, 12 aa, Homo sapiens
ACDLLGDTDKLI

SEQ ID NO: 542 - protein, 15 aa, Homo sapiens
ACDSTGGSWDTRQMF

SEQ ID NO: 543 - protein, 12 aa, Homo sapiens
ACDTLGDTDKLI

SEQ ID NO: 544 - protein, 16 aa, Homo sapiens
ACDVLGDSSWDTRQMF

SEQ ID NO: 545 - protein, 14 aa, Homo sapiens
ACDWGNSWDTRQMF

SEQ ID NO: 546 - protein, 14 aa, Homo sapiens
ACDWGSSWDTRQTF

SEQ ID NO: 547 - protein, 14 aa, Homo sapiens
ACDWGSAWDTRQMF

SEQ ID NO: 548 - protein, 14 aa, Homo sapiens
ACDWGSSCDTRQMF

SEQ ID NO: 549 - protein, 12 aa, Homo sapiens
ACGTGGYTDKLI

SEQ ID NO: 550 - protein, 12 aa, Homo sapiens
ACDAGGYTDKLI

SEQ ID NO: 551 - protein, 14 aa, Homo sapiens
ACDWGSSWDTRLMF

SEQ ID NO: 552 - protein, 14 aa, Homo sapiens
ACDGGSSWDTRQMF

SEQ ID NO: 553 - protein, 14 aa, Homo sapiens
GCDWGSSWDTRQMF

SEQ ID NO: 554 - protein, 14 aa, Homo sapiens
ACDWESSWDTRQMF

SEQ ID NO: 555 - protein, 14 aa, Homo sapiens
ACDLGSSWDTRQMF

SEQ ID NO: 556 - protein, 14 aa, Homo sapiens
ACDWGSSWDTREMF

SEQ ID NO: 557 - protein, 14 aa, Homo sapiens
ACDVLGDTLTAQLF

SEQ ID NO: 558 - protein, 15 aa, Homo sapiens
ACDTAGGSWDTRQMF

SEQ ID NO: 559 - protein, 13 aa, Homo sapiens
ACDVLGDLTAQLF

SEQ ID NO: 560 - protein, 16 aa, Homo sapiens
ACDNTGGYSWDTRQMF

SEQ ID NO: 561 - protein, 16 aa, Homo sapiens
ACDTVGGYSWDTRQMF

SEQ ID NO: 562 - protein, 13 aa, Homo sapiens
AFTGGYWDTRQMF

SEQ ID NO: 563 - protein, 15 aa, Homo sapiens
ACDTAGGYWDTRQMF

SEQ ID NO: 564 - protein, 11 aa, Homo sapiens
AFTGGYTDKLI

SEQ ID NO: 565 - protein, 14 aa, Homo sapiens
ACDTVGDTLTAQLF

SEQ ID NO: 566 - protein, 12 aa, Homo sapiens
ACDTWGLTAQLF

SEQ ID NO: 567 - protein, 15 aa, Homo sapiens
ACDWGIRSWDTRQMF

SEQ ID NO: 568 - protein, 16 aa, Homo sapiens
ACDTLGDSSWDTRQMF

SEQ ID NO: 569 - protein, 12 aa, Homo sapiens
ACDTGVYTDKLI

SEQ ID NO: 570 - protein, 12 aa, Homo sapiens
ACDSGGYTDKLI

SEQ ID NO: 571 - protein, 12 aa, Homo sapiens
ACDTGGYSDKLI

SEQ ID NO: 572 - protein, 14 aa, Homo sapiens
TCDWGSSWDTRQMF

SEQ ID NO: 573 - protein, 12 aa, Homo sapiens
ACDTGGHTDKLI

SEQ ID NO: 574 - protein, 12 aa, Homo sapiens
ARDTGGYTDKLI

SEQ ID NO: 575 - protein, 14 aa, Homo sapiens
ACDWGSSWDTRQLF

SEQ ID NO: 576 - protein, 14 aa, Homo sapiens
ACDWGSSWDTRQIF

SEQ ID NO: 577 - protein, 14 aa, Homo sapiens
ACDWGSSWDSRQMF

SEQ ID NO: 578 - protein, 12 aa, Homo sapiens
ACDTGGCTDKLI

SEQ ID NO: 579 - protein, 14 aa, Homo sapiens
ACDWGSCWDTRQMF

SEQ ID NO: 580 - protein, 14 aa, Homo sapiens
ACDWRSSWDTRQMF

SEQ ID NO: 581 - protein, 14 aa, Homo sapiens
AFDWGSSWDTRQMF

SEQ ID NO: 582 - protein, 14 aa, Homo sapiens
ACDWGSSWDTRQMV

SEQ ID NO: 583 - protein, 12 aa, Homo sapiens
ACDTGGYAAQLF

SEQ ID NO: 584 - protein, 16 aa, Homo sapiens
ACDILGDSSWDTRQMF

SEQ ID NO: 585 - protein, 14 aa, Homo sapiens
AFTGGYSWDTRQMF

SEQ ID NO: 586 - protein, 10 aa, Homo sapiens
AFGGYTDKLI

SEQ ID NO: 587 - protein, 16 aa, Homo sapiens
ACDTGGYASWDTRQMF

SEQ ID NO: 588 - protein, 13 aa, Homo sapiens
ACDILGDTTAQLF

SEQ ID NO: 589 - protein, 14 aa, Homo sapiens
ALWEVQELGKKIKV

SEQ ID NO: 590 - protein, 11 aa, Homo sapiens
ATWDTTGWFKI

SEQ ID NO: 591 - protein, 9 aa, Homo sapiens
ATWDYYKKL

SEQ ID NO: 592 - protein, 10 aa, Homo sapiens
ATWDGYYKKL

SEQ ID NO: 593 - protein, 11 aa, Homo sapiens
ATWDGNYYKKL

SEQ ID NO: 594 - protein, 11 aa, Homo sapiens
ATWDGPYYKKL

SEQ ID NO: 595 - protein, 8 aa, Homo sapiens
ATWDYKKL

SEQ ID NO: 596 - protein, 9 aa, Homo sapiens
ATWDGYKKL

SEQ ID NO: 597 - protein, 10 aa, Homo sapiens
ATWDGRYKKL

SEQ ID NO: 598 - protein, 12 aa, Homo sapiens
ATWDGPNYYKKL

SEQ ID NO: 599 - protein, 10 aa, Homo sapiens
ATWDGPYKKL

SEQ ID NO: 600 - protein, 11 aa, Homo sapiens
ATWDGLYYKKL

SEQ ID NO: 601 - protein, 7 aa, Homo sapiens
ATWDKKL

SEQ ID NO: 602 - protein, 11 aa, Homo sapiens
ATWDGRYYKKL

SEQ ID NO: 603 - protein, 10 aa, Homo sapiens
ATWDGHYKKL

SEQ ID NO: 604 - protein, 11 aa, Homo sapiens
ATWDRPYYKKL

SEQ ID NO: 605 - protein, 11 aa, Homo sapiens
ALWEVNYYKKL

SEQ ID NO: 606 - protein, 11 aa, Homo sapiens
ATWDSSDWIKT

SEQ ID NO: 607 - protein, 12 aa, Homo sapiens
ATWDGPGYYKKL

SEQ ID NO: 608 - protein, 10 aa, Homo sapiens
ALWEVYYKKL

SEQ ID NO: 609 - protein, 9 aa, Homo sapiens
ATWNYYKKL

SEQ ID NO: 610 - protein, 11 aa, Homo sapiens
ATWDGSDWIKT

SEQ ID NO: 611 - protein, 12 aa, Homo sapiens
ATWDGSSDWIKT

SEQ ID NO: 612 - protein, 10 aa, Homo sapiens
ATWDGGYKKL

SEQ ID NO: 613 - protein, 10 aa, Homo sapiens
ATWENYYKKL

SEQ ID NO: 614 - protein, 9 aa, Homo sapiens
ATWDDYKKL

SEQ ID NO: 615 - protein, 9 aa, Homo sapiens
ATWGYYKKL

SEQ ID NO: 616 - protein, 10 aa, Homo sapiens
ATWDGLYKKL

SEQ ID NO: 617 - protein, 11 aa, Homo sapiens
ATWDATGWFKI

SEQ ID NO: 618 - protein, 12 aa, Homo sapiens
ATWDGRNYYKKL

SEQ ID NO: 619 - protein, 9 aa, Homo sapiens
ATWDGRKKL

SEQ ID NO: 620 - protein, 11 aa, Homo sapiens
ATWDGPGWFKI

SEQ ID NO: 621 - protein, 9 aa, Homo sapiens
ATWDSYKKL

SEQ ID NO: 622 - protein, 11 aa, Homo sapiens
ATWDRLYYKKL

SEQ ID NO: 623 - protein, 11 aa, Homo sapiens
ATWDGLGYKKL

SEQ ID NO: 624 - protein, 11 aa, Homo sapiens
ATWDGFYYKKL

SEQ ID NO: 625 - protein, 10 aa, Homo sapiens
ATWDLYYKKL

SEQ ID NO: 626 - protein, 13 aa, Homo sapiens
ATWDGYSSDWIKT

SEQ ID NO: 627 - protein, 11 aa, Homo sapiens
ATWDGGYYKKL

SEQ ID NO: 628 - protein, 10 aa, Homo sapiens
ATWVNYYKKL

SEQ ID NO: 629 - protein, 12 aa, Homo sapiens
ATWDGPSDWIKT

SEQ ID NO: 630 - protein, 8 aa, Homo sapiens
ATWYYKKL

SEQ ID NO: 631 - protein, 11 aa, Homo sapiens
ATWDSNYYKKL

SEQ ID NO: 632 - protein, 11 aa, Homo sapiens
ATWDGTYYKKL

SEQ ID NO: 633 - protein, 12 aa, Homo sapiens
ATWDGRDYYKKL

SEQ ID NO: 634 - protein, 12 aa, Homo sapiens
ATWDGQNYYKKL

SEQ ID NO: 635 - protein, 7 aa, Homo sapiens
ATWDEKL

SEQ ID NO: 636 - protein, 11 aa, Homo sapiens
ATWDSTGWFKI

SEQ ID NO: 637 - protein, 9 aa, Homo sapiens
ATCDYYKKL

SEQ ID NO: 638 - protein, 11 aa, Homo sapiens
ATWDGPGYKKL

SEQ ID NO: 639 - protein, 9 aa, Homo sapiens
ATWDGPKKL

SEQ ID NO: 640 - protein, 11 aa, Homo sapiens
ATWDRNYYKKL

SEQ ID NO: 641 - protein, 10 aa, Homo sapiens
ATWDSYYKKL

SEQ ID NO: 642 - protein, 8 aa, Homo sapiens
ATWDGKKL

SEQ ID NO: 643 - protein, 9 aa, Homo sapiens
ATWDRYKKL

SEQ ID NO: 644 - protein, 11 aa, Homo sapiens
ATWDGDYYKKL

SEQ ID NO: 645 - protein, 12 aa, Homo sapiens
ATWDGPTGWFKI

SEQ ID NO: 646 - protein, 11 aa, Homo sapiens
ATWDRRYYKKL

SEQ ID NO: 647 - protein, 11 aa, Homo sapiens
ATWDGPRYKKL

SEQ ID NO: 648 - protein, 9 aa, Homo sapiens
ATWDGWFKI

SEQ ID NO: 649 - protein, 10 aa, Homo sapiens
ATWDDYYKKL

SEQ ID NO: 650 - protein, 11 aa, Homo sapiens
ATWDGSYYKKL

SEQ ID NO: 651 - protein, 12 aa, Homo sapiens
ATWDRPNYYKKL

SEQ ID NO: 652 - protein, 10 aa, Homo sapiens
ATWDRYYKKL

SEQ ID NO: 653 - protein, 10 aa, Homo sapiens
ATWDGNYKKL

SEQ ID NO: 654 - protein, 11 aa, Homo sapiens
ATWDRPGYKKL

SEQ ID NO: 655 - protein, 12 aa, Homo sapiens
ATWDGSNYYKKL

SEQ ID NO: 656 - protein, 11 aa, Homo sapiens
ATWDGRGYKKL

SEQ ID NO: 657 - protein, 9 aa, Homo sapiens
ATWDGPEKL

SEQ ID NO: 658 - protein, 11 aa, Homo sapiens
ATWDGQGYKKL

SEQ ID NO: 659 - protein, 12 aa, Homo sapiens
ATWDGLNYYKKL

SEQ ID NO: 660 - protein, 11 aa, Homo sapiens
ATWDENYYKKL

SEQ ID NO: 661 - protein, 11 aa, Homo sapiens
ATWDGYYYKKL

SEQ ID NO: 662 - protein, 9 aa, Homo sapiens
ATWDGDKKL

SEQ ID NO: 663 - protein, 11 aa, Homo sapiens
ATWDGPNYKKL

SEQ ID NO: 664 - protein, 13 aa, Homo sapiens
ATWDGYTTGWFKI

SEQ ID NO: 665 - protein, 10 aa, Homo sapiens
ATWGNYYKKL

SEQ ID NO: 666 - protein, 10 aa, Homo sapiens
ATWDVYYKKL

SEQ ID NO: 667 - protein, 11 aa, Homo sapiens
ATWDGTGWFKI

SEQ ID NO: 668 - protein, 10 aa, Homo sapiens
ALWEDYYKKL

SEQ ID NO: 669 - protein, 11 aa, Homo sapiens
ATWDRRDYKKL

SEQ ID NO: 670 - protein, 10 aa, Homo sapiens
ATWDPYYKKL

SEQ ID NO: 671 - protein, 11 aa, Homo sapiens
ATWDGIYYKKL

SEQ ID NO: 672 - protein, 11 aa, Homo sapiens
ATWDGRDYKKL

SEQ ID NO: 673 - protein, 11 aa, Homo sapiens
ATWDGQYYKKL

SEQ ID NO: 674 - protein, 11 aa, Homo sapiens
ATWDGVYYKKL

SEQ ID NO: 675 - protein, 8 aa, Homo sapiens
ATWGYKKL

SEQ ID NO: 676 - protein, 8 aa, Homo sapiens
ALWEVKKL

SEQ ID NO: 677 - protein, 10 aa, Homo sapiens
ATWDGSYKKL

SEQ ID NO: 678 - protein, 9 aa, Homo sapiens
ATWEYYKKL

SEQ ID NO: 679 - protein, 10 aa, Homo sapiens
ALWEVGYKKL

SEQ ID NO: 680 - protein, 11 aa, Homo sapiens
ATWDRGYYKKL

SEQ ID NO: 681 - protein, 12 aa, Homo sapiens
ATWDGPHYYKKL

SEQ ID NO: 682 - protein, 9 aa, Homo sapiens
ATWDRRGKL

SEQ ID NO: 683 - protein, 12 aa, Homo sapiens
ATWDGRGYYKKL

SEQ ID NO: 684 - protein, 12 aa, Homo sapiens
ATWDRRNYYKKL

SEQ ID NO: 685 - protein, 11 aa, Homo sapiens
ATWDRPRYKKL

SEQ ID NO: 686 - protein, 11 aa, Homo sapiens
ATWDGPVYKKL

SEQ ID NO: 687 - protein, 6 aa, Homo sapiens
ARDLDY

SEQ ID NO: 688 - protein, 6 aa, Homo sapiens
ARDFDY

SEQ ID NO: 689 - protein, 5 aa, Homo sapiens
ARDDY

SEQ ID NO: 690 - protein, 6 aa, Homo sapiens
ARGFDY

SEQ ID NO: 691 - protein, 12 aa, Homo sapiens
ARDSSGWYYFDY

SEQ ID NO: 692 - protein, 5 aa, Homo sapiens
AREDY

SEQ ID NO: 693 - protein, 8 aa, Homo sapiens
ARGNWFDP

SEQ ID NO: 694 - protein, 6 aa, Homo sapiens
ARDPDY

SEQ ID NO: 695 - protein, 6 aa, Homo sapiens
ARDLGY

SEQ ID NO: 696 - protein, 6 aa, Homo sapiens
ARGLDY

SEQ ID NO: 697 - protein, 12 aa, Homo sapiens
ARIGYSSSSFDY

SEQ ID NO: 698 - protein, 8 aa, Homo sapiens
ARGDWFDP

SEQ ID NO: 699 - protein, 5 aa, Homo sapiens
ARVDY

SEQ ID NO: 700 - protein, 11 aa, Homo sapiens
ARDYYYYGMDV

SEQ ID NO: 701 - protein, 5 aa, Homo sapiens
ARGDY

SEQ ID NO: 702 - protein, 10 aa, Homo sapiens
ARDYYYGMDV

SEQ ID NO: 703 - protein, 7 aa, Homo sapiens
ARDPFDY

SEQ ID NO: 704 - protein, 7 aa, Homo sapiens
ARDAFDI

SEQ ID NO: 705 - protein, 9 aa, Homo sapiens
ARGHYGMDV

SEQ ID NO: 706 - protein, 8 aa, Homo sapiens
ARGDAFDI

SEQ ID NO: 707 - protein, 13 aa, Homo sapiens
ARGYSSSWYYFDY

SEQ ID NO: 708 - protein, 8 aa, Homo sapiens
ARDYGMDV

SEQ ID NO: 709 - protein, 8 aa, Homo sapiens
ARDDAFDI

SEQ ID NO: 710 - protein, 6 aa, Homo sapiens
ARGVDY

SEQ ID NO: 711 - protein, 6 aa, Homo sapiens
ARNFDY

SEQ ID NO: 712 - protein, 6 aa, Homo sapiens
ARDGDY

SEQ ID NO: 713 - protein, 6 aa, Homo sapiens
ARGIDY

SEQ ID NO: 714 - protein, 9 aa, Homo sapiens
ARGYYGMDV

SEQ ID NO: 715 - protein, 13 aa, Homo sapiens
ARGYSSGWYYFDY

SEQ ID NO: 716 - protein, 6 aa, Homo sapiens
AREFDY

SEQ ID NO: 717 - protein, 11 aa, Homo sapiens
ARGDYYYGMDV

SEQ ID NO: 718 - protein, 9 aa, Homo sapiens
ARGRYYFDY

SEQ ID NO: 719 - protein, 5 aa, Homo sapiens
ARLDY

SEQ ID NO: 720 - protein, 6 aa, Homo sapiens
AKDLDY

SEQ ID NO: 721 - protein, 9 aa, Homo sapiens
ARGGYYFDY

SEQ ID NO: 722 - protein, 5 aa, Homo sapiens
AREGY

SEQ ID NO: 723 - protein, 11 aa, Homo sapiens
ARGYYYYGMDV

SEQ ID NO: 724 - protein, 7 aa, Homo sapiens
ARGWFDP

SEQ ID NO: 725 - protein, 15 aa, Homo sapiens
ARDYYDSSGYYYFDY

SEQ ID NO: 726 - protein, 10 aa, Homo sapiens
ARGYYYGMDV

SEQ ID NO: 727 - protein, 6 aa, Homo sapiens
ARDSDY

SEQ ID NO: 728 - protein, 8 aa, Homo sapiens
ARGYGMDV

SEQ ID NO: 729 - protein, 9 aa, Homo sapiens
ARDRGYFDY

SEQ ID NO: 730 - protein, 6 aa, Homo sapiens
ARGSDY

SEQ ID NO: 731 - protein, 6 aa, Homo sapiens
ARELDY

SEQ ID NO: 732 - protein, 8 aa, Homo sapiens
ARGYYFDY

SEQ ID NO: 733 - protein, 9 aa, Homo sapiens
ARGLYYFDY

SEQ ID NO: 734 - protein, 9 aa, Homo sapiens
ARGHYGLDV

SEQ ID NO: 735 - protein, 6 aa, Homo sapiens
ARDFGY

SEQ ID NO: 736 - protein, 8 aa, Homo sapiens
ARDNWFDP

SEQ ID NO: 737 - protein, 12 aa, Homo sapiens
ARDSSSWYYFDY

SEQ ID NO: 738 - protein, 13 aa, Homo sapiens
ARDYGGNSGWFDP

SEQ ID NO: 739 - protein, 9 aa, Homo sapiens
ARDSYGMDV

SEQ ID NO: 740 - protein, 5 aa, Homo sapiens
ARDGY

SEQ ID NO: 741 - protein, 11 aa, Homo sapiens
ARDYGDYYFDY

SEQ ID NO: 742 - protein, 6 aa, Homo sapiens
ARDIDY

SEQ ID NO: 743 - protein, 9 aa, Homo sapiens
ARDRGWFDP

SEQ ID NO: 744 - protein, 6 aa, Homo sapiens
ARVFDY

SEQ ID NO: 745 - protein, 7 aa, Homo sapiens
ARDLGDY

SEQ ID NO: 746 - protein, 12 aa, Homo sapiens
ARGSSGWYYFDY

SEQ ID NO: 747 - protein, 8 aa, Homo sapiens
ARGRWFDP

SEQ ID NO: 748 - protein, 9 aa, Homo sapiens
ARGEYYFDY

SEQ ID NO: 749 - protein, 9 aa, Homo sapiens
ARDYYGMDV

SEQ ID NO: 750 - protein, 11 aa, Homo sapiens
ARDSGSYYFDY

SEQ ID NO: 751 - protein, 10 aa, Homo sapiens
ARDYGDYFDY

SEQ ID NO: 752 - protein, 6 aa, Homo sapiens
ARAFDY

SEQ ID NO: 753 - protein, 9 aa, Homo sapiens
ARGRNWFDP

SEQ ID NO: 754 - protein, 12 aa, Homo sapiens
ARDYYGDYYFDY

SEQ ID NO: 755 - protein, 8 aa, Homo sapiens
ARDRWFDP

SEQ ID NO: 756 - protein, 12 aa, Homo sapiens
ARGDYYYYGMDV

SEQ ID NO: 757 - protein, 6 aa, Homo sapiens
ARDVDY

SEQ ID NO: 758 - protein, 7 aa, Homo sapiens
ARDGFDY

SEQ ID NO: 759 - protein, 6 aa, Homo sapiens
ARSFDY

SEQ ID NO: 760 - protein, 5 aa, Homo sapiens
AKDDY

SEQ ID NO: 761 - protein, 13 aa, Homo sapiens
ARDYYDSSGYFDY

SEQ ID NO: 762 - protein, 5 aa, Homo sapiens
ASLDY

SEQ ID NO: 763 - protein, 15 aa, Homo sapiens
ARGYYDSSGYYYFDY

SEQ ID NO: 764 - protein, 12 aa, Homo sapiens
ARIGYSSSSLDY

SEQ ID NO: 765 - protein, 6 aa, Homo sapiens
ARDYDY

SEQ ID NO: 766 - protein, 7 aa, Homo sapiens
ARGAFDI

SEQ ID NO: 767 - protein, 5 aa, Homo sapiens
ARVGY

SEQ ID NO: 768 - protein, 8 aa, Homo sapiens
ARDYYFDY

SEQ ID NO: 769 - protein, 9 aa, Homo sapiens
ARDRDAFDI

SEQ ID NO: 770 - protein, 5 aa, Homo sapiens
TTVDY

SEQ ID NO: 771 - protein, 7 aa, Homo sapiens
ARDPGDY

SEQ ID NO: 772 - protein, 6 aa, Homo sapiens
ARGGDY

SEQ ID NO: 773 - protein, 6 aa, Homo sapiens
ARDRGY

SEQ ID NO: 774 - protein, 14 aa, Homo sapiens
ARGYCSGGSCYFDY

SEQ ID NO: 775 - protein, 6 aa, Homo sapiens
ARDRDY

SEQ ID NO: 776 - protein, 5 aa, Homo sapiens
ARGGY

SEQ ID NO: 777 - protein, 13 aa, Homo sapiens
ARAYSSGWYYFDY

SEQ ID NO: 778 - protein, 6 aa, Homo sapiens
ARDPGY

SEQ ID NO: 779 - protein, 8 aa, Homo sapiens
ARGGWFDP

SEQ ID NO: 780 - protein, 8 aa, Homo sapiens
ARGPPFDY

SEQ ID NO: 781 - protein, 8 aa, Homo sapiens
ARGGAFDI

SEQ ID NO: 782 - protein, 7 aa, Homo sapiens
ARGPFDY

SEQ ID NO: 783 - protein, 6 aa, Homo sapiens
ARGYDY

SEQ ID NO: 784 - protein, 11 aa, Homo sapiens
ARDSSGWYFDY

SEQ ID NO: 785 - protein, 9 aa, Homo sapiens
QQYDNLPLT

SEQ ID NO: 786 - protein, 9 aa, Homo sapiens
QQYGSSPRT

SEQ ID NO: 787 - protein, 9 aa, Homo sapiens
QQSYSTPRT

SEQ ID NO: 788 - protein, 9 aa, Homo sapiens
QQYYSTPYT

SEQ ID NO: 789 - protein, 9 aa, Homo sapiens
QQYGSSPWT

SEQ ID NO: 790 - protein, 9 aa, Homo sapiens
QQSYSTPYT

SEQ ID NO: 791 - protein, 10 aa, Homo sapiens [sequence omitted]

SEQ ID NO: 792 - protein, 9 aa, Homo sapiens
QQYGSSPLT

SEQ ID NO: 793 - protein, 9 aa, Homo sapiens
QQYGSSPYT

SEQ ID NO: 794 - protein, 9 aa, Homo sapiens
QQSYSTPWT

SEQ ID NO: 795 - protein, 9 aa, Homo sapiens
QQYNNWPRT

SEQ ID NO: 796 - protein, 9 aa, Homo sapiens
QQRSNWPLT

SEQ ID NO: 797 - protein, 9 aa, Homo sapiens
QQYNSYPWT

SEQ ID NO: 798 - protein, 9 aa, Homo sapiens
QQSYSTPLT

SEQ ID NO: 799 - protein, 9 aa, Homo sapiens
QQYDNLPYT

SEQ ID NO: 800 - protein, 10 aa, Homo sapiens
QQYNNWPPYT

SEQ ID NO: 801 - protein, 10 aa, Homo sapiens
QQYNNWPPLT

SEQ ID NO: 802 - protein, 9 aa, Homo sapiens
QQYNSYPYT

SEQ ID NO: 803 - protein, 9 aa, Homo sapiens
QQYNSYPLT

SEQ ID NO: 804 - protein, 9 aa, Homo sapiens
QQYYSYPRT

SEQ ID NO: 805 - protein, 9 aa, Homo sapiens
QQYNNWPLT

SEQ ID NO: 806 - protein, 10 aa, Homo sapiens
QQYGSSPPYT

SEQ ID NO: 807 - protein, 9 aa, Homo sapiens
QQSYSTPPT

SEQ ID NO: 808 - protein, 10 aa, Homo sapiens
QQRSNWPPIT

SEQ ID NO: 809 - protein, 9 aa, Homo sapiens
QQRSNWPPT

SEQ ID NO: 810 - protein, 9 aa, Homo sapiens
QQYNSYSWT

SEQ ID NO: 811 - protein, 9 aa, Homo sapiens
LQHNSYPWT

SEQ ID NO: 812 - protein, 9 aa, Homo sapiens
QQYYSTPLT

SEQ ID NO: 813 - protein, 9 aa, Homo sapiens
QQYGSSPFT

SEQ ID NO: 814 - protein, 9 aa, Homo sapiens
QQYDNLPIT

SEQ ID NO: 815 - protein, 9 aa, Homo sapiens
QQRSNWPRT

SEQ ID NO: 816 - protein, 9 aa, Homo sapiens
QQYNSYSRT

SEQ ID NO: 817 - protein, 9 aa, Homo sapiens
QQYGSSPIT

SEQ ID NO: 818 - protein, 10 aa, Homo sapiens
QQRSNWPPLT

SEQ ID NO: 819 - protein, 9 aa, Homo sapiens
LQHNSYPRT

SEQ ID NO: 820 - protein, 10 aa, Homo sapiens
QQRSNWPPYT

SEQ ID NO: 821 - protein, 10 aa, Homo sapiens
QQYGSSPPWT

SEQ ID NO: 822 - protein, 8 aa, Homo sapiens
QQYNSYWT

SEQ ID NO: 823 - protein, 9 aa, Homo sapiens
QQYGSSPQT

SEQ ID NO: 824 - protein, 9 aa, Homo sapiens
QQYGSSPGT

SEQ ID NO: 825 - protein, 9 aa, Homo sapiens
QQLNSYPLT

SEQ ID NO: 826 - protein, 9 aa, Homo sapiens
QQSYSTPFT

SEQ ID NO: 827 - protein, 9 aa, Homo sapiens
QQYDNLPPT

SEQ ID NO: 828 - protein, 10 aa, Homo sapiens
QQYNNWPPIT

SEQ ID NO: 829 - protein, 10 aa, Homo sapiens
QQRSNWPPWT

SEQ ID NO: 830 - protein, 9 aa, Homo sapiens
QQYDNLPFT

SEQ ID NO: 831 - protein, 9 aa, Homo sapiens
MQALQTPRT

SEQ ID NO: 832 - protein, 9 aa, Homo sapiens
QQYGSSLWT

SEQ ID NO: 833 - protein, 8 aa, Homo sapiens
QQYGSSPT

SEQ ID NO: 834 - protein, 9 aa, Homo sapiens
MQGTHWPYT

SEQ ID NO: 835 - protein, 9 aa, Homo sapiens
LQHNSYPLT

SEQ ID NO: 836 - protein, 9 aa, Homo sapiens
QQRSNWPIT

SEQ ID NO: 837 - protein, 9 aa, Homo sapiens
MQALQTPLT

SEQ ID NO: 838 - protein, 9 aa, Homo sapiens
QQYGSSPPT

SEQ ID NO: 839 - protein, 9 aa, Homo sapiens
QQYDNLPRT

SEQ ID NO: 840 - protein, 9 aa, Homo sapiens
QQYYSTPRT

SEQ ID NO: 841 - protein, 9 aa, Homo sapiens
QQYYSYPLT

SEQ ID NO: 842 - protein, 9 aa, Homo sapiens
MQALQTPYT

SEQ ID NO: 843 - protein, 10 aa, Homo sapiens
QQYGSSPPIT

SEQ ID NO: 844 - protein, 9 aa, Homo sapiens
QQYNNWPYT

SEQ ID NO: 845 - protein, 9 aa, Homo sapiens
MQALQTPWT

SEQ ID NO: 846 - protein, 9 aa, Homo sapiens
QQSYSTPIT

SEQ ID NO: 847 - protein, 9 aa, Homo sapiens
QQANSFPLT

SEQ ID NO: 848 - protein, 9 aa, Homo sapiens
QQYYSTPWT

SEQ ID NO: 849 - protein, 9 aa, Homo sapiens
QQSYSTPQT

SEQ ID NO: 850 - protein, 9 aa, Homo sapiens
QQYNSYSYT

SEQ ID NO: 851 - protein, 9 aa, Homo sapiens
QQLNSYPRT

SEQ ID NO: 852 - protein, 9 aa, Homo sapiens
LQDYNYPRT

SEQ ID NO: 853 - protein, 9 aa, Homo sapiens
QQYYSYPWT

SEQ ID NO: 854 - protein, 9 aa, Homo sapiens
QQSYSTLWT

SEQ ID NO: 855 - protein, 8 aa, Homo sapiens
QQYGSSRT

SEQ ID NO: 856 - protein, 9 aa, Homo sapiens
QQRSNWPWT

SEQ ID NO: 857 - protein, 9 aa, Homo sapiens
QQYNSYPRT

SEQ ID NO: 858 - protein, 10 aa, Homo sapiens
QQYGSSPPLT

SEQ ID NO: 859 - protein, 9 aa, Homo sapiens
QQYYSTPPT

SEQ ID NO: 860 - protein, 8 aa, Homo sapiens
QQYNSYST

SEQ ID NO: 861 - protein, 9 aa, Homo sapiens
LQHNSYPYT

SEQ ID NO: 862 - protein, 10 aa, Homo sapiens
QQYGSSPLYT

SEQ ID NO: 863 - protein, 9 aa, Homo sapiens
QQYNNWPWT

SEQ ID NO: 864 - protein, 9 aa, Homo sapiens
LQDYNYPWT

SEQ ID NO: 865 - protein, 8 aa, Homo sapiens
QQRSNWPT

SEQ ID NO: 866 - protein, 10 aa, Homo sapiens
QQYDNLPPYT

SEQ ID NO: 867 - protein, 10 aa, Homo sapiens
QQSYSTPPYT

SEQ ID NO: 868 - protein, 9 aa, Homo sapiens
QQYGSSLYT

SEQ ID NO: 869 - protein, 9 aa, Homo sapiens
QQYGSSSWT

SEQ ID NO: 870 - protein, 10 aa, Homo sapiens
QQYGSSPLFT

SEQ ID NO: 871 - protein, 9 aa, Homo sapiens
QQYYSYPYT

SEQ ID NO: 872 - protein, 9 aa, Homo sapiens
QQYNSYPFT

SEQ ID NO: 873 - protein, 9 aa, Homo sapiens
QQYGSSPKT

SEQ ID NO: 874 - protein, 9 aa, Homo sapiens
MQALQTPPT

SEQ ID NO: 875 - protein, 9 aa, Homo sapiens
QQRSNWPYT

SEQ ID NO: 876 - protein, 10 aa, Homo sapiens
QQRSNWPPFT

SEQ ID NO: 877 - protein, 9 aa, Homo sapiens
QQSYTTPRT

SEQ ID NO: 878 - protein, 9 aa, Homo sapiens
QQYNNWPQT

SEQ ID NO: 879 - protein, 9 aa, Homo sapiens
QQYYSYPPT

SEQ ID NO: 880 - protein, 10 aa, Homo sapiens
QQYDNLPPLT

SEQ ID NO: 881 - protein, 9 aa, Homo sapiens
QQYNSYPIT

SEQ ID NO: 882 - protein, 9 aa, Homo sapiens
QQLNSYPFT

SEQ ID NO: 883 - protein, 11 aa, Homo sapiens
GTWDSSLSAGV

SEQ ID NO: 884 - protein, 11 aa, Homo sapiens
GTWDSSLSAVV

SEQ ID NO: 885 - protein, 11 aa, Homo sapiens
QVWDSSSDHVV

SEQ ID NO: 886 - protein, 10 aa, Homo sapiens
SSYTSSSTLV

SEQ ID NO: 887 - protein, 11 aa, Homo sapiens
QSYDSSLSGSV

SEQ ID NO: 888 - protein, 11 aa, Homo sapiens
QSADSSGTYVV

SEQ ID NO: 889 - protein, 11 aa, Homo sapiens
GTWDSSLSAWV

SEQ ID NO: 890 - protein, 11 aa, Homo sapiens
QSYDSSLSGWV

SEQ ID NO: 891 - protein, 11 aa, Homo sapiens
SSYTSSSTLVV

SEQ ID NO: 892 - protein, 10 aa, Homo sapiens
SSYTSSSTVV

SEQ ID NO: 893 - protein, 11 aa, Homo sapiens
NSRDSSGNHLV

SEQ ID NO: 894 - protein, 11 aa, Homo sapiens
AAWDDSLNGPV

SEQ ID NO: 895 - protein, 10 aa, Homo sapiens
SSYTSSSTWV

SEQ ID NO: 896 - protein, 11 aa, Homo sapiens
QSADSSGTYWV

SEQ ID NO: 897 - protein, 11 aa, Homo sapiens
NSRDSSGNHWV

SEQ ID NO: 898 - protein, 9 aa, Homo sapiens
QAWDSSTVV

SEQ ID NO: 899 - protein, 11 aa, Homo sapiens
NSRDSSGNHVV

SEQ ID NO: 900 - protein, 10 aa, Homo sapiens
SSYTSSSTRV

SEQ ID NO: 901 - protein, 10 aa, Homo sapiens
SSYAGSNNLV

SEQ ID NO: 902 - protein, 11 aa, Homo sapiens
GTWDSSLSVVV

SEQ ID NO: 903 - protein, 11 aa, Homo sapiens
QVWDSSSDHPV

SEQ ID NO: 904 - protein, 10 aa, Homo sapiens
CSYAGSSTLV

SEQ ID NO: 905 - protein, 11 aa, Homo sapiens
QSYDSSLSGYV

SEQ ID NO: 906 - protein, 10 aa, Homo sapiens
CSYAGSSTWV

SEQ ID NO: 907 - protein, 9 aa, Homo sapiens
MIWHSSAWV

SEQ ID NO: 908 - protein, 12 aa, Homo sapiens
QSYDSSLSGSRV

SEQ ID NO: 909 - protein, 12 aa, Homo sapiens
GTWDSSLSAGGV

SEQ ID NO: 910 - protein, 10 aa, Homo sapiens
QSADSSGTWV

SEQ ID NO: 911 - protein, 11 aa, Homo sapiens
GTWDSSLSAEV

SEQ ID NO: 912 - protein, 10 aa, Homo sapiens
CSYAGSYTWV

SEQ ID NO: 913 - protein, 11 aa, Homo sapiens
QVWDSSSDHRV

SEQ ID NO: 914 - protein, 10 aa, Homo sapiens
GTWDSSLSAV

SEQ ID NO: 915 - protein, 11 aa, Homo sapiens
QSADSSGTYRV

SEQ ID NO: 916 - protein, 11 aa, Homo sapiens
NSRDSSGNHRV

SEQ ID NO: 917 - protein, 11 aa, Homo sapiens
GTWDNSLSAGV

SEQ ID NO: 918 - protein, 12 aa, Homo sapiens
QSYDSSLSGSVV

SEQ ID NO: 919 - protein, 11 aa, Homo sapiens
GTWDSSLSAYV

SEQ ID NO: 920 - protein, 11 aa, Homo sapiens
QSYDSSLSGVV

SEQ ID NO: 921 - protein, 9 aa, Homo sapiens
QTWGTGIRV

SEQ ID NO: 922 - protein, 11 aa, Homo sapiens
AAWDDSLNGRV

SEQ ID NO: 923 - protein, 11 aa, Homo sapiens
AAWDDSLSGPV

SEQ ID NO: 924 - protein, 11 aa, Homo sapiens
SSYTSSSTLGV

SEQ ID NO: 925 - protein, 12 aa, Homo sapiens
GTWDSSLSAGRV

SEQ ID NO: 926 - protein, 11 aa, Homo sapiens
CSYAGSSTFVV

SEQ ID NO: 927 - protein, 11 aa, Homo sapiens
GTWDSSLSARV

SEQ ID NO: 928 - protein, 11 aa, Homo sapiens
AAWDDSLSGRV

SEQ ID NO: 929 - protein, 10 aa, Homo sapiens
GTWDSSLSVV

SEQ ID NO: 930 - protein, 11 aa, Homo sapiens
GTWDSSLSAAV

SEQ ID NO: 931 - protein, 11 aa, Homo sapiens
SSYTSSSTLEV

SEQ ID NO: 932 - protein, 10 aa, Homo sapiens
CSYAGSSTYV

SEQ ID NO: 933 - protein, 10 aa, Homo sapiens
SSYTSSSTPV

SEQ ID NO: 934 - protein, 11 aa, Homo sapiens
GTWDGSLSAGV

SEQ ID NO: 935 - protein, 11 aa, Homo sapiens
QVWDSSSDLVV

SEQ ID NO: 936 - protein, 11 aa, Homo sapiens
GTWDSSLSALV

SEQ ID NO: 937 - protein, 11 aa, Homo sapiens
GTWDSSLSGGV

SEQ ID NO: 938 - protein, 11 aa, Homo sapiens
QSYDSSLSGRV

SEQ ID NO: 939 - protein, 11 aa, Homo sapiens
QSYDSSLSGLV

SEQ ID NO: 940 - protein, 11 aa, Homo sapiens
GTWDSSLRAGV

SEQ ID NO: 941 - protein, 11 aa, Homo sapiens
GAWDSSLSAVV

SEQ ID NO: 942 - protein, 9 aa, Homo sapiens
SSYTSSSTV

SEQ ID NO: 943 - protein, 11 aa, Homo sapiens
GTWDSSLRAVV

SEQ ID NO: 944 - protein, 11 aa, Homo sapiens
GTWDSGLSAGV

SEQ ID NO: 945 - protein, 11 aa, Homo sapiens
GAWDSSLSAGV

SEQ ID NO: 946 - protein, 11 aa, Homo sapiens
GTWDRSLSAVV

SEQ ID NO: 947 - protein, 11 aa, Homo sapiens
QSYDSSLSGGV

SEQ ID NO: 948 - protein, 11 aa, Homo sapiens
GTWDSSLNAGV

SEQ ID NO: 949 - protein, 11 aa, Homo sapiens
GTWDSRLSAGV

SEQ ID NO: 950 - protein, 11 aa, Homo sapiens
GTWDRSLSAGV

SEQ ID NO: 951 - protein, 11 aa, Homo sapiens
QSYDSSLSGAV

SEQ ID NO: 952 - protein, 11 aa, Homo sapiens
GTWDSSLSAGG

SEQ ID NO: 953 - protein, 11 aa, Homo sapiens
GSWDSSLSAGV

SEQ ID NO: 954 - protein, 11 aa, Homo sapiens
GTWDSRLSAVV

SEQ ID NO: 955 - protein, 11 aa, Homo sapiens
GTWDSSLGAGV

SEQ ID NO: 956 - protein, 11 aa, Homo sapiens
GSWDSSLSAVV

SEQ ID NO: 957 - protein, 11 aa, Homo sapiens
VTWDSSLSAGV

SEQ ID NO: 958 - protein, 11 aa, Homo sapiens
QSYDSSLRGSV

SEQ ID NO: 959 - protein, 11 aa, Homo sapiens
GTWDSSLSAGA

SEQ ID NO: 960 - protein, 11 aa, Homo sapiens
GTWGSSLSAGV

SEQ ID NO: 961 - protein, 11 aa, Homo sapiens
QSYDSSLGGSV

SEQ ID NO: 962 - protein, 11 aa, Homo sapiens
GTWGSSLSAVV

SEQ ID NO: 963 - protein, 11 aa, Homo sapiens
GTRDSSLSAVV

SEQ ID NO: 964 - protein, 11 aa, Homo sapiens
QSYDSGLSGSV

SEQ ID NO: 965 - protein, 10 aa, Homo sapiens
GSYTSSSTLV

SEQ ID NO: 966 - protein, 11 aa, Homo sapiens
GTRDSSLSAGV

SEQ ID NO: 967 - protein, 11 aa, Homo sapiens
GTWDSSPSAGV

SEQ ID NO: 968 - protein, 11 aa, Homo sapiens
GTWDSSPSAVV

SEQ ID NO: 969 - protein, 11 aa, Homo sapiens
RTWDSSLSAGV

SEQ ID NO: 970 - protein, 10 aa, Homo sapiens
GTDSSLSAGV

SEQ ID NO: 971 - protein, 11 aa, Homo sapiens
GTWDSSLCAVV

SEQ ID NO: 972 - protein, 11 aa, Homo sapiens
GTWDSCLSAVV

SEQ ID NO: 973 - protein, 11 aa, Homo sapiens
GTWDSSLCAGV

SEQ ID NO: 974 - protein, 11 aa, Homo sapiens
GTWDSCLSAGV

SEQ ID NO: 975 - protein, 11 aa, Homo sapiens
GTWVSSLSAVV

SEQ ID NO: 976 - protein, 11 aa, Homo sapiens
GTWVSSLSAGV

SEQ ID NO: 977 - protein, 11 aa, Homo sapiens
GTCDSSLSAGV

SEQ ID NO: 978 - protein, 11 aa, Homo sapiens
QVWDSSSDHWV

SEQ ID NO: 979 - protein, 11 aa, Homo sapiens
AAWDDSLNGWV

SEQ ID NO: 980 - protein, 11 aa, Homo sapiens
GTWDSSLSVGV

SEQ ID NO: 981 - protein, 10 aa, Homo sapiens
AVRDSNYQLI

SEQ ID NO: 982 - protein, 10 aa, Homo sapiens
AVMDSNYQLI

SEQ ID NO: 983 - protein, 10 aa, Homo sapiens
AVLDSNYQLI

SEQ ID NO: 984 - protein, 10 aa, Homo sapiens
AVKDSNYQLI

SEQ ID NO: 985 - protein, 10 aa, Homo sapiens
AVVDSNYQLI

SEQ ID NO: 986 - protein, 10 aa, Homo sapiens
AVTDSNYQLI

SEQ ID NO: 987 - protein, 10 aa, Homo sapiens
AVIDSNYQLI

SEQ ID NO: 988 - protein, 10 aa, Homo sapiens
AVNTGGFKTI

SEQ ID NO: 989 - protein, 10 aa, Homo sapiens
AVNQAGTALI

SEQ ID NO: 990 - protein, 10 aa, Homo sapiens
AVNTGFQKLV

SEQ ID NO: 991 - protein, 8 aa, Homo sapiens
AVNDYKLS

SEQ ID NO: 992 - protein, 11 aa, Homo sapiens
AVNSGGYQKVT

SEQ ID NO: 993 - protein, 10 aa, Homo sapiens
AVTSGTYKYI

SEQ ID NO: 994 - protein, 9 aa, Homo sapiens
AVDSNYQLI

SEQ ID NO: 995 - protein, 10 aa, Homo sapiens
AVNTNAGKST

SEQ ID NO: 996 - protein, 9 aa, Homo sapiens
AVNRDDKII

SEQ ID NO: 997 - protein, 9 aa, Homo sapiens
AVNTGNQFY

SEQ ID NO: 998 - protein, 8 aa, Homo sapiens
AVRDDKII

SEQ ID NO: 999 - protein, 10 aa, Homo sapiens
AVGGSQGNLI

SEQ ID NO: 1000 - protein, 12 aa, Homo sapiens
AVNSGGSNYKLT

SEQ ID NO: 1001 - protein, 10 aa, Homo sapiens
AVSGSARQLT

SEQ ID NO: 1002 - protein, 10 aa, Homo sapiens
AVGDSNYQLI

SEQ ID NO: 1003 - protein, 10 aa, Homo sapiens
AVDTGRRALT

SEQ ID NO: 1004 - protein, 10 aa, Homo sapiens
AVNNAGNMLT

SEQ ID NO: 1005 - protein, 12 aa, Homo sapiens
AVNYGGSQGNLI

SEQ ID NO: 1006 - protein, 10 aa, Homo sapiens
AVYTGGFKTI

SEQ ID NO: 1007 - protein, 9 aa, Homo sapiens
AASNDYKLS

SEQ ID NO: 1008 - protein, 10 aa, Homo sapiens
AVNSGYSTLT

SEQ ID NO: 1009 - protein, 10 aa, Homo sapiens
AANQAGTALI

SEQ ID NO: 1010 - protein, 11 aa, Homo sapiens
AASGGSNYKLT

SEQ ID NO: 1011 - protein, 10 aa, Homo sapiens
AVSGGSYIPT

SEQ ID NO: 1012 - protein, 10 aa, Homo sapiens
ALMDSNYQLI

SEQ ID NO: 1013 - protein, 10 aa, Homo sapiens
ALNTGGFKTI

SEQ ID NO: 1014 - protein, 10 aa, Homo sapiens
AANFGNEKLT

SEQ ID NO: 1015 - protein, 11 aa, Homo sapiens
AASTSGTYKYI

SEQ ID NO: 1016 - protein, 8 aa, Homo sapiens
AVNTDKLI

SEQ ID NO: 1017 - protein, 8 aa, Homo sapiens
AVTGNQFY

SEQ ID NO: 1018 - protein, 11 aa, Homo sapiens
AVLNQAGTALI

SEQ ID NO: 1019 - protein, 10 aa, Homo sapiens
AASGGSYIPT

SEQ ID NO: 1020 - protein, 10 aa, Homo sapiens
AVKAAGNKLT

SEQ ID NO: 1021 - protein, 11 aa, Homo sapiens
AVNAGNNRKLI

SEQ ID NO: 1022 - protein, 11 aa, Homo sapiens
AVSGGSNYKLT

SEQ ID NO: 1023 - protein, 13 aa, Homo sapiens
AANAGGTSYGKLT

SEQ ID NO: 1024 - protein, 8 aa, Homo sapiens
ALNDYKLS

SEQ ID NO: 1025 - protein, 10 aa, Homo sapiens
AVSGGYQKVT

SEQ ID NO: 1026 - protein, 10 aa, Homo sapiens
AVDTGGFKTI

SEQ ID NO: 1027 - protein, 11 aa, Homo sapiens
AVRNTGGFKTI

SEQ ID NO: 1028 - protein, 10 aa, Homo sapiens
AVRSNDYKLS

SEQ ID NO: 1029 - protein, 10 aa, Homo sapiens
AVSSNDYKLS

SEQ ID NO: 1030 - protein, 10 aa, Homo sapiens
AVTGTASKLT

SEQ ID NO: 1031 - protein, 11 aa, Homo sapiens
AVNTGTASKLT

SEQ ID NO: 1032 - protein, 12 aa, Homo sapiens
AVAGGTSYGKLT

SEQ ID NO: 1033 - protein, 11 aa, Homo sapiens
AVTTSGTYKYI

SEQ ID NO: 1034 - protein, 10 aa, Homo sapiens
AGGGSQGNLI

SEQ ID NO: 1035 - protein, 10 aa, Homo sapiens
AVHTGGFKTI

SEQ ID NO: 1036 - protein, 9 aa, Homo sapiens
AVYNTDKLI

SEQ ID NO: 1037 - protein, 9 aa, Homo sapiens
VVNTGNQFY

SEQ ID NO: 1038 - protein, 11 aa, Homo sapiens
AVSSGSARQLT

SEQ ID NO: 1039 - protein, 10 aa, Homo sapiens
AARDSNYQLI

SEQ ID NO: 1040 - protein, 10 aa, Homo sapiens
AANNAGNMLT

SEQ ID NO: 1041 - protein, 11 aa, Homo sapiens
AVSNFGNEKLT

SEQ ID NO: 1042 - protein, 11 aa, Homo sapiens
AVSDTGGFKTI

SEQ ID NO: 1043 - protein, 12 aa, Homo sapiens
AVDRGSTLGRLY

SEQ ID NO: 1044 - protein, 11 aa, Homo sapiens
AASSGSARQLT

SEQ ID NO: 1045 - protein, 10 aa, Homo sapiens
ALGGSQGNLI

SEQ ID NO: 1046 - protein, 11 aa, Homo sapiens
AASSGGYQKVT

SEQ ID NO: 1047 - protein, 11 aa, Homo sapiens
AVSNTGGFKTI

SEQ ID NO: 1048 - protein, 13 aa, Homo sapiens
AASAGGTSYGKLT

SEQ ID NO: 1049 - protein, 13 aa, Homo sapiens
AVSAGGTSYGKLT

SEQ ID NO: 1050 - protein, 10 aa, Homo sapiens
AVMDSSYKLI

SEQ ID NO: 1051 - protein, 10 aa, Homo sapiens
AAMDSNYQLI

SEQ ID NO: 1052 - protein, 10 aa, Homo sapiens
AVSDSNYQLI

SEQ ID NO: 1053 - protein, 10 aa, Homo sapiens
AALDSNYQLI

SEQ ID NO: 1054 - protein, 10 aa, Homo sapiens
AVEDSNYQLI

SEQ ID NO: 1055 - protein, 10 aa, Homo sapiens
AASDSNYQLI

SEQ ID NO: 1056 - protein, 9 aa, Homo sapiens
AVSNDYKLS

SEQ ID NO: 1057 - protein, 11 aa, Homo sapiens
AASKGGSYIPT

SEQ ID NO: 1058 - protein, 11 aa, Homo sapiens
AVPNQAGTALI

SEQ ID NO: 1059 - protein, 10 aa, Homo sapiens
AVNFGNEKLT

SEQ ID NO: 1060 - protein, 11 aa, Homo sapiens
AVRDNYGQNFV

SEQ ID NO: 1061 - protein, 10 aa, Homo sapiens
AAYTGGFKTI

SEQ ID NO: 1062 - protein, 13 aa, Homo sapiens
AVNAGGTSYGKLT

SEQ ID NO: 1063 - protein, 11 aa, Homo sapiens
ALSGGSNYKLT

SEQ ID NO: 1064 - protein, 9 aa, Homo sapiens
ALYNFNKFY

SEQ ID NO: 1065 - protein, 10 aa, Homo sapiens
AVYSSASKII

SEQ ID NO: 1066 - protein, 10 aa, Homo sapiens
AGGTSYGKLT

SEQ ID NO: 1067 - protein, 11 aa, Homo sapiens
AVASGGSYIPT

SEQ ID NO: 1068 - protein, 10 aa, Homo sapiens
VVNTNAGKST

SEQ ID NO: 1069 - protein, 10 aa, Homo sapiens
AVNSGNTPLV

SEQ ID NO: 1070 - protein, 11 aa, Homo sapiens
AVSNQAGTALI

SEQ ID NO: 1071 - protein, 12 aa, Homo sapiens
AENSGGSNYKLT

SEQ ID NO: 1072 - protein, 10 aa, Homo sapiens
AVRGSQGNLI

SEQ ID NO: 1073 - protein, 11 aa, Homo sapiens
AASNQAGTALI

SEQ ID NO: 1074 - protein, 10 aa, Homo sapiens
AAGGSQGNLI

SEQ ID NO: 1075 - protein, 10 aa, Homo sapiens
AVQTGANNLF

SEQ ID NO: 1076 - protein, 9 aa, Homo sapiens
AVYNFNKFY

SEQ ID NO: 1077 - protein, 10 aa, Homo sapiens
AVKTSYDKVI

SEQ ID NO: 1078 - protein, 11 aa, Homo sapiens
AVFTGGGNKLT

SEQ ID NO: 1079 - protein, 9 aa, Homo sapiens
ASSLNTEAF

SEQ ID NO: 1080 - protein, 11 aa, Homo sapiens
ASSLGGNTEAF

SEQ ID NO: 1081 - protein, 10 aa, Homo sapiens
ASSPSTDTQY

SEQ ID NO: 1082 - protein, 9 aa, Homo sapiens
ASSLTDTQY

SEQ ID NO: 1083 - protein, 11 aa, Homo sapiens
ASSLGQNTEAF

SEQ ID NO: 1084 - protein, 9 aa, Homo sapiens
ASSLSYEQY

SEQ ID NO: 1085 - protein, 11 aa, Homo sapiens
ASSLQGNTEAF

SEQ ID NO: 1086 - protein, 10 aa, Homo sapiens
ASSLGGTEAF

SEQ ID NO: 1087 - protein, 11 aa, Homo sapiens
ASSLGSNQPQH

SEQ ID NO: 1088 - protein, 11 aa, Homo sapiens
ASSLDSNQPQH

SEQ ID NO: 1089 - protein, 11 aa, Homo sapiens
ASSLGGNQPQH

SEQ ID NO: 1090 - protein, 9 aa, Homo sapiens
ASSLQETQY

SEQ ID NO: 1091 - protein, 9 aa, Homo sapiens
ASSLGYEQY

SEQ ID NO: 1092 - protein, 10 aa, Homo sapiens
ASSLGNTEAF

SEQ ID NO: 1093 - protein, 11 aa, Homo sapiens
ASSLGRNTEAF

SEQ ID NO: 1094 - protein, 11 aa, Homo sapiens
ASSLGLNTEAF

SEQ ID NO: 1095 - protein, 9 aa, Homo sapiens
ASSSTDTQY

SEQ ID NO: 1096 - protein, 10 aa, Homo sapiens
ASSLSTDTQY

SEQ ID NO: 1097 - protein, 9 aa, Homo sapiens
ASSPSYEQY

SEQ ID NO: 1098 - protein, 11 aa, Homo sapiens
ASSLTGNTEAF

SEQ ID NO: 1099 - protein, 9 aa, Homo sapiens
ASSSSYEQY

SEQ ID NO: 1100 - protein, 10 aa, Homo sapiens
ASSLGNQPQH

SEQ ID NO: 1101 - protein, 11 aa, Homo sapiens
ASSLDRNTEAF

SEQ ID NO: 1102 - protein, 10 aa, Homo sapiens
ASSLGTDTQY

SEQ ID NO: 1103 - protein, 9 aa, Homo sapiens
ASSFTDTQY

SEQ ID NO: 1104 - protein, 10 aa, Homo sapiens
ASSLGGYEQY

SEQ ID NO: 1105 - protein, 9 aa, Homo sapiens
ASSLGTEAF

SEQ ID NO: 1106 - protein, 10 aa, Homo sapiens
ASSLGSYEQY

SEQ ID NO: 1107 - protein, 9 aa, Homo sapiens
ASSLYNEQF

SEQ ID NO: 1108 - protein, 10 aa, Homo sapiens
ASSLSSYEQY

SEQ ID NO: 1109 - protein, 9 aa, Homo sapiens
ASSSQETQY

SEQ ID NO: 1110 - protein, 9 aa, Homo sapiens
ASSSYNEQF

SEQ ID NO: 1111 - protein, 11 aa, Homo sapiens
ASSLTVNTEAF

SEQ ID NO: 1112 - protein, 9 aa, Homo sapiens
ASSLGETQY

SEQ ID NO: 1113 - protein, 9 aa, Homo sapiens
ASSPQETQY

SEQ ID NO: 1114 - protein, 11 aa, Homo sapiens
ASSLGGSYEQY

SEQ ID NO: 1115 - protein, 9 aa, Homo sapiens
ASSLEETQY

SEQ ID NO: 1116 - protein, 9 aa, Homo sapiens
ASSYSYEQY

SEQ ID NO: 1117 - protein, 8 aa, Homo sapiens
ASSQETQY

SEQ ID NO: 1118 - protein, 10 aa, Homo sapiens
ASSLSNQPQH

SEQ ID NO: 1119 - protein, 11 aa, Homo sapiens
ASSLGGTDTQY

SEQ ID NO: 1120 - protein, 11 aa, Homo sapiens
ASSLGVNTEAF

SEQ ID NO: 1121 - protein, 12 aa, Homo sapiens
ASSLGQGNQPQH

SEQ ID NO: 1122 - protein, 11 aa, Homo sapiens
ASSLGPNTEAF

SEQ ID NO: 1123 - protein, 9 aa, Homo sapiens
ASSLADTQY

SEQ ID NO: 1124 - protein, 11 aa, Homo sapiens
ASSLGGGTEAF

SEQ ID NO: 1125 - protein, 11 aa, Homo sapiens
ASSLAGNTEAF

SEQ ID NO: 1126 - protein, 9 aa, Homo sapiens
ASSLGDTQY

SEQ ID NO: 1127 - protein, 10 aa, Homo sapiens
ASSSSTDTQY

SEQ ID NO: 1128 - protein, 11 aa, Homo sapiens
ASSLTGGTEAF

SEQ ID NO: 1129 - protein, 10 aa, Homo sapiens
ASSPSSYEQY

SEQ ID NO: 1130 - protein, 11 aa, Homo sapiens
ASSLGQNYGYT

SEQ ID NO: 1131 - protein, 10 aa, Homo sapiens
ASSFSTDTQY

SEQ ID NO: 1132 - protein, 12 aa, Homo sapiens
ASSLGQGNTEAF

SEQ ID NO: 1133 - protein, 12 aa, Homo sapiens
ASSLGGSNQPQH

SEQ ID NO: 1134 - protein, 12 aa, Homo sapiens
ASSPGQGNQPQH

SEQ ID NO: 1135 - protein, 12 aa, Homo sapiens
ASSLGGGNQPQH

SEQ ID NO: 1136 - protein, 11 aa, Homo sapiens
ASSLAGNQPQH

SEQ ID NO: 1137 - protein, 9 aa, Homo sapiens
ASSRNTEAF

SEQ ID NO: 1138 - protein, 11 aa, Homo sapiens
ASSLRGNTEAF

SEQ ID NO: 1139 - protein, 9 aa, Homo sapiens
ASSFSYEQY

SEQ ID NO: 1140 - protein, 9 aa, Homo sapiens
ASSQGYEQY

SEQ ID NO: 1141 - protein, 11 aa, Homo sapiens
ASSLQGNQPQH

SEQ ID NO: 1142 - protein, 12 aa, Homo sapiens
ASSLAGGTDTQY

SEQ ID NO: 1143 - protein, 10 aa, Homo sapiens
ASSLLNTEAF

SEQ ID NO: 1144 - protein, 11 aa, Homo sapiens
ASSRDSNQPQH

SEQ ID NO: 1145 - protein, 12 aa, Homo sapiens
ASSLGGSTDTQY

SEQ ID NO: 1146 - protein, 9 aa, Homo sapiens
ASSFQETQY

SEQ ID NO: 1147 - protein, 10 aa, Homo sapiens
ASSLMNTEAF

SEQ ID NO: 1148 - protein, 10 aa, Homo sapiens
ASSLGGYGYT

SEQ ID NO: 1149 - protein, 11 aa, Homo sapiens
ASSLYSNQPQH

SEQ ID NO: 1150 - protein, 11 aa, Homo sapiens
ASSPGQNTEAF

SEQ ID NO: 1151 - protein, 11 aa, Homo sapiens
ASSPDRNTEAF

SEQ ID NO: 1152 - protein, 11 aa, Homo sapiens
ASSLVGNTEAF

SEQ ID NO: 1153 - protein, 12 aa, Homo sapiens
ASSLGGSSYEQY

SEQ ID NO: 1154 - protein, 11 aa, Homo sapiens
ASSPPSTDTQY

SEQ ID NO: 1155 - protein, 11 aa, Homo sapiens
ASSRQGNTEAF

SEQ ID NO: 1156 - protein, 11 aa, Homo sapiens
ASSLGQGYGYT

SEQ ID NO: 1157 - protein, 11 aa, Homo sapiens
ASSLGSSYEQY

SEQ ID NO: 1158 - protein, 9 aa, Homo sapiens
ASSRTDTQY

SEQ ID NO: 1159 - protein, 11 aa, Homo sapiens
ASSQDSNQPQH

SEQ ID NO: 1160 - protein, 11 aa, Homo sapiens
ASSLTGNQPQH

SEQ ID NO: 1161 - protein, 11 aa, Homo sapiens
ASSPGQGYEQY

SEQ ID NO: 1162 - protein, 11 aa, Homo sapiens
ASSLGSTDTQY

SEQ ID NO: 1163 - protein, 11 aa, Homo sapiens
ASSLEGNTEAF

SEQ ID NO: 1164 - protein, 12 aa, Homo sapiens
ASSLGQLNTEAF

SEQ ID NO: 1165 - protein, 11 aa, Homo sapiens
ASSLGQGYEQY

SEQ ID NO: 1166 - protein, 9 aa, Homo sapiens
ASSYTDTQY

SEQ ID NO: 1167 - protein, 11 aa, Homo sapiens
ASSQGLNTEAF

SEQ ID NO: 1168 - protein, 10 aa, Homo sapiens
ASSLDSYEQY

SEQ ID NO: 1169 - protein, 10 aa, Homo sapiens
ASSLGGQPQH

SEQ ID NO: 1170 - protein, 11 aa, Homo sapiens
ASSLTENTEAF

SEQ ID NO: 1171 - protein, 11 aa, Homo sapiens
ASSLNSNQPQH

SEQ ID NO: 1172 - protein, 11 aa, Homo sapiens
ASSLSGNTEAF

SEQ ID NO: 1173 - protein, 12 aa, Homo sapiens
ASSLGQGAYEQY

SEQ ID NO: 1174 - protein, 11 aa, Homo sapiens
ASSLEGNQPQH

SEQ ID NO: 1175 - protein, 9 aa, Homo sapiens
ASSLKETQY

SEQ ID NO: 1176 - protein, 11 aa, Homo sapiens
ASSLAGGTEAF

SEQ ID NO: 1177 - protein, 12 aa, Homo sapiens
ACDTLGDTDKLI

SEQ ID NO: 1178 - protein, 13 aa, Homo sapiens
ACDTVGGYTDKLI

SEQ ID NO: 1179 - protein, 12 aa, Homo sapiens
ACDTVGDTDKLI

SEQ ID NO: 1180 - protein, 12 aa, Homo sapiens
ACDRLGDTDKLI

SEQ ID NO: 1181 - protein, 12 aa, Homo sapiens
ACDILGDTDKLI

SEQ ID NO: 1182 - protein, 13 aa, Homo sapiens
ACDTLLGDTDKLI

SEQ ID NO: 1183 - protein, 12 aa, Homo sapiens
ACDPLGDTDKLI

SEQ ID NO: 1184 - protein, 13 aa, Homo sapiens
ACDPLLGDTDKLI

SEQ ID NO: 1185 - protein, 12 aa, Homo sapiens
ACDTVGGTDKLI

SEQ ID NO: 1186 - protein, 12 aa, Homo sapiens
ACDVLGDTDKLI

SEQ ID NO: 1187 - protein, 12 aa, Homo sapiens
ACDTLGGTDKLI

SEQ ID NO: 1188 - protein, 12 aa, Homo sapiens
ACDSLGDTDKLI

SEQ ID NO: 1189 - protein, 12 aa, Homo sapiens
ACDLLGDTDKLI

SEQ ID NO: 1190 - protein, 12 aa, Homo sapiens
ACDALGDTDKLI

SEQ ID NO: 1191 - protein, 13 aa, Homo sapiens
ACDTVGEYTDKLI

SEQ ID NO: 1192 - protein, 16 aa, Homo sapiens
ACDTAGGSSWDTRQMF

SEQ ID NO: 1193 - protein, 13 aa, Homo sapiens
ACDTVGGSTDKLI

SEQ ID NO: 1194 - protein, 13 aa, Homo sapiens
ACDSLLGDTDKLI

SEQ ID NO: 1195 - protein, 13 aa, Homo sapiens
ACDTLGDTSDKLI

SEQ ID NO: 1196 - protein, 13 aa, Homo sapiens
ACDTVGGNTDKLI

SEQ ID NO: 1197 - protein, 12 aa, Homo sapiens
ACDTLGYTDKLI

SEQ ID NO: 1198 - protein, 18 aa, Homo sapiens
ACDTVLGDTSSWDTRQMF

SEQ ID NO: 1199 - protein, 13 aa, Homo sapiens
ACDTVGTYTDKLI

SEQ ID NO: 1200 - protein, 17 aa, Homo sapiens
ACDTVLGDSSWDTRQMF

SEQ ID NO: 1201 - protein, 13 aa, Homo sapiens
ACDTVGSYTDKLI

SEQ ID NO: 1202 - protein, 13 aa, Homo sapiens
ACDALLGDTDKLI

SEQ ID NO: 1203 - protein, 13 aa, Homo sapiens
ACDTVGAYTDKLI

SEQ ID NO: 1204 - protein, 12 aa, Homo sapiens
ACDKLGDTDKLI

SEQ ID NO: 1205 - protein, 13 aa, Homo sapiens
ACDTVGGHTDKLI

SEQ ID NO: 1206 - protein, 12 aa, Homo sapiens
ACDTVGGSDKLI

SEQ ID NO: 1207 - protein, 12 aa, Homo sapiens
ACDTLGDADKLI

SEQ ID NO: 1208 - protein, 13 aa, Homo sapiens
ACDTLGGYTDKLI

SEQ ID NO: 1209 - protein, 12 aa, Homo sapiens
ACDTLGDSDKLI

SEQ ID NO: 1210 - protein, 12 aa, Homo sapiens
ACDTVGYTDKLI

SEQ ID NO: 1211 - protein, 13 aa, Homo sapiens
ACDTVGVYTDKLI

SEQ ID NO: 1212 - protein, 12 aa, Homo sapiens
ACGTLGDTDKLI

SEQ ID NO: 1213 - protein, 14 aa, Homo sapiens
ACDTVGGSYTDKLI

SEQ ID NO: 1214 - protein, 13 aa, Homo sapiens
ACDTLGAYTDKLI

SEQ ID NO: 1215 - protein, 13 aa, Homo sapiens
ACDTVGGRTDKLI

SEQ ID NO: 1216 - protein, 14 aa, Homo sapiens
ACDTLGDTGTDKLI

SEQ ID NO: 1217 - protein, 16 aa, Homo sapiens
ACDTGGYGSWDTRQMF

SEQ ID NO: 1218 - protein, 12 aa, Homo sapiens
ARDTLGDTDKLI

SEQ ID NO: 1219 - protein, 16 aa, Homo sapiens
ACDTVLGDTRYTDKLI

SEQ ID NO: 1220 - protein, 12 aa, Homo sapiens
ACDTLGVTDKLI

SEQ ID NO: 1221 - protein, 13 aa, Homo sapiens
ACDTVGGYADKLI

SEQ ID NO: 1222 - protein, 13 aa, Homo sapiens
ACDTVGGDTDKLI

SEQ ID NO: 1223 - protein, 12 aa, Homo sapiens
ACDTLGETDKLI

SEQ ID NO: 1224 - protein, 12 aa, Homo sapiens
ACDTLGDTDKLT

SEQ ID NO: 1225 - protein, 12 aa, Homo sapiens
ACDTLGDTDKPI

SEQ ID NO: 1226 - protein, 13 aa, Homo sapiens
ACDTLGTYTDKLI

SEQ ID NO: 1227 - protein, 14 aa, Homo sapiens
ACDTLGDTRTDKLI

SEQ ID NO: 1228 - protein, 17 aa, Homo sapiens
ACDTVLGDTSWDTRQMF

SEQ ID NO: 1229 - protein, 13 aa, Homo sapiens
ACDTLGEYTDKLI

SEQ ID NO: 1230 - protein, 13 aa, Homo sapiens
ACDTLGVYTDKLI

SEQ ID NO: 1231 - protein, 13 aa, Homo sapiens
ACDTLGANTDKLI

SEQ ID NO: 1232 - protein, 13 aa, Homo sapiens
ACDSVGGYTDKLI

SEQ ID NO: 1233 - protein, 13 aa, Homo sapiens
ACDTLGDTADKLI

SEQ ID NO: 1234 - protein, 12 aa, Homo sapiens
ACDTWGNTDKLI

SEQ ID NO: 1235 - protein, 14 aa, Homo sapiens
ACDTLGDTYTDKLI

SEQ ID NO: 1236 - protein, 13 aa, Homo sapiens
ACDTVGENTDKLI

SEQ ID NO: 1237 - protein, 12 aa, Homo sapiens
ACDTLGNTDKLI

SEQ ID NO: 1238 - protein, 15 aa, Homo sapiens
ACDTLLGDTYTDKLI

SEQ ID NO: 1239 - protein, 11 aa, Homo sapiens
ACDTWGTDKLI

SEQ ID NO: 1240 - protein, 14 aa, Homo sapiens
ACDTVGGLYTDKLI

SEQ ID NO: 1241 - protein, 13 aa, Homo sapiens
ACDTVGGFTDKLI

SEQ ID NO: 1242 - protein, 13 aa, Homo sapiens
ACDTLGGNTDKLI

SEQ ID NO: 1243 - protein, 13 aa, Homo sapiens
ACDTVGPYTDKLI

SEQ ID NO: 1244 - protein, 15 aa, Homo sapiens
ACDTAGGSWDTRQMF

SEQ ID NO: 1245 - protein, 13 aa, Homo sapiens
ACDPLGDYTDKLI

SEQ ID NO: 1246 - protein, 14 aa, Homo sapiens
ACDTVGGPYTDKLI

SEQ ID NO: 1247 - protein, 13 aa, Homo sapiens
ACDTLGENTDKLI

SEQ ID NO: 1248 - protein, 13 aa, Homo sapiens
ACDTVGLYTDKLI

SEQ ID NO: 1249 - protein, 14 aa, Homo sapiens
ACDTLGDTDTDKLI

SEQ ID NO: 1250 - protein, 12 aa, Homo sapiens
ACDTWGDTDKLI

SEQ ID NO: 1251 - protein, 12 aa, Homo sapiens
ACDTVGVTDKLI

SEQ ID NO: 1252 - protein, 12 aa, Homo sapiens
ACDTLGDTVKLI

SEQ ID NO: 1253 - protein, 14 aa, Homo sapiens
ACDTVGGTYTDKLI

SEQ ID NO: 1254 - protein, 13 aa, Homo sapiens
ACDTVGANTDKLI

SEQ ID NO: 1255 - protein, 14 aa, Homo sapiens
ACDTLGDSYTDKLI

SEQ ID NO: 1256 - protein, 16 aa, Homo sapiens
ACDTLLGDTRYTDKLI

SEQ ID NO: 1257 - protein, 12 aa, Homo sapiens
ACDTVLGTDKLI

SEQ ID NO: 1258 - protein, 14 aa, Homo sapiens
ACDTLGGPYTDKLI

SEQ ID NO: 1259 - protein, 13 aa, Homo sapiens
ACDTVGGLTDKLI

SEQ ID NO: 1260 - protein, 12 aa, Homo sapiens
ACDTLGDTGKLI

SEQ ID NO: 1261 - protein, 18 aa, Homo sapiens
ACDTVLGDTRSWDTRQMF

SEQ ID NO: 1262 - protein, 14 aa, Homo sapiens
ACDTLGDPYTDKLI

SEQ ID NO: 1263 - protein, 12 aa, Homo sapiens
ACDTLGATDKLI

SEQ ID NO: 1264 - protein, 13 aa, Homo sapiens
ACDTVGGGTDKLI

SEQ ID NO: 1265 - protein, 16 aa, Homo sapiens
ACDTVLGDTWDTRQMF

SEQ ID NO: 1266 - protein, 13 aa, Homo sapiens
ACDTLGDTRDKLI

SEQ ID NO: 1267 - protein, 15 aa, Homo sapiens
ACDTTGGSWDTRQMF

SEQ ID NO: 1268 - protein, 14 aa, Homo sapiens
ACDTVGGRYTDKLI

SEQ ID NO: 1269 - protein, 13 aa, Homo sapiens
ACDPLGGYTDKLI

SEQ ID NO: 1270 - protein, 14 aa, Homo sapiens
ACDTVGGGYTDKLI

SEQ ID NO: 1271 - protein, 12 aa, Homo sapiens
ACDTWGYTDKLI

SEQ ID NO: 1272 - protein, 16 aa, Homo sapiens
ACDTGGYSSWDTRQMF

SEQ ID NO: 1273 - protein, 12 aa, Homo sapiens
ACDTVGDSDKLI

SEQ ID NO: 1274 - protein, 14 aa, Homo sapiens
ACDTLLGDTTDKLI

SEQ ID NO: 1275 - protein, 14 aa, Homo sapiens
ALWEVQELGKKIKV

SEQ ID NO: 1276 - protein, 14 aa, Homo sapiens
ALWEVRELGKKIKV

SEQ ID NO: 1277 - protein, 14 aa, Homo sapiens
ALWEVLELGKKIKV

SEQ ID NO: 1278 - protein, 10 aa, Homo sapiens
ATWDGYYKKL

SEQ ID NO: 1279 - protein, 11 aa, Homo sapiens
ATWDGNYYKKL

SEQ ID NO: 1280 - protein, 14 aa, Homo sapiens
ALWEVQEFGKKIKV

SEQ ID NO: 1281 - protein, 14 aa, Homo sapiens
ALWEGQELGKKIKV

SEQ ID NO: 1282 - protein, 14 aa, Homo sapiens
ALWEAQELGKKIKV

SEQ ID NO: 1283 - protein, 9 aa, Homo sapiens
ATWDYYKKL

SEQ ID NO: 1284 - protein, 11 aa, Homo sapiens
ATWDGPYYKKL

SEQ ID NO: 1285 - protein, 14 aa, Homo sapiens
ALWDVQELGKKIKV

SEQ ID NO: 1286 - protein, 14 aa, Homo sapiens
ALWEVQELCKKIKV

SEQ ID NO: 1287 - protein, 14 aa, Homo sapiens
ALWEVQELGKIIKV

SEQ ID NO: 1288 - protein, 14 aa, Homo sapiens
ALGEVQELGKKIKV

SEQ ID NO: 1289 - protein, 14 aa, Homo sapiens
ALWEVKELGKKIKV

SEQ ID NO: 1290 - protein, 8 aa, Homo sapiens
ATWDYKKL

SEQ ID NO: 1291 - protein, 14 aa, Homo sapiens
ALWEVGELGKKIKV

SEQ ID NO: 1292 - protein, 14 aa, Homo sapiens
ALWEEQELGKKIKV

SEQ ID NO: 1293 - protein, 11 aa, Homo sapiens
ATWDGLYYKKL

SEQ ID NO: 1294 - protein, 14 aa, Homo sapiens
ALWEVQELVKKIKV

SEQ ID NO: 1295 - protein, 14 aa, Homo sapiens
ALWEVHELGKKIKV

SEQ ID NO: 1296 - protein, 10 aa, Homo sapiens
ATWDGRYKKL

SEQ ID NO: 1297 - protein, 14 aa, Homo sapiens
ALWEVEELGKKIKV

SEQ ID NO: 1298 - protein, 14 aa, Homo sapiens
ALWVVQELGKKIKV

SEQ ID NO: 1299 - protein, 7 aa, Homo sapiens
ATWDKKL

SEQ ID NO: 1300 - protein, 13 aa, Homo sapiens
ALWEEELGKKIKV

SEQ ID NO: 1301 - protein, 12 aa, Homo sapiens
ATWDGPNYYKKL

SEQ ID NO: 1302 - protein, 11 aa, Homo sapiens
ATWDGRYYKKL

SEQ ID NO: 1303 - protein, 11 aa, Homo sapiens
ATWDRNYYKKL

SEQ ID NO: 1304 - protein, 14 aa, Homo sapiens
ALWEGRELGKKIKV

SEQ ID NO: 1305 - protein, 14 aa, Homo sapiens
GLWEVQELGKKIKV

SEQ ID NO: 1306 - protein, 14 aa, Homo sapiens
ALWEVREFGKKIKV

SEQ ID NO: 1307 - protein, 14 aa, Homo sapiens
ALWEDQELGKKIKV

SEQ ID NO: 1308 - protein, 11 aa, Homo sapiens
ATWDGPGYKKL

SEQ ID NO: 1309 - protein, 14 aa, Homo sapiens
ALWEVQGLGKKIKV

SEQ ID NO: 1310 - protein, 14 aa, Homo sapiens
ALWEVQEVGKKIKV

SEQ ID NO: 1311 - protein, 10 aa, Homo sapiens
ATWDGPYKKL

SEQ ID NO: 1312 - protein, 13 aa, Homo sapiens
ALWEQELGKKIKV

SEQ ID NO: 1313 - protein, 14 aa, Homo sapiens
ALWELQELGKKIKV

SEQ ID NO: 1314 - protein, 14 aa, Homo sapiens
ALWEVQVLGKKIKV

SEQ ID NO: 1315 - protein, 14 aa, Homo sapiens
ALCEVQELGKKIKV

SEQ ID NO: 1316 - protein, 14 aa, Homo sapiens
ALREVQELGKKIKV

SEQ ID NO: 1317 - protein, 10 aa, Homo sapiens
ALWEVRYKKL

SEQ ID NO: 1318 - protein, 14 aa, Homo sapiens
ALWETQELGKKIKV

SEQ ID NO: 1319 - protein, 11 aa, Homo sapiens
ALWEVNYYKKL

SEQ ID NO: 1320 - protein, 12 aa, Homo sapiens
ATWDGLNYYKKL

SEQ ID NO: 1321 - protein, 14 aa, Homo sapiens
ALWESQELGKKIKV

SEQ ID NO: 1322 - protein, 10 aa, Homo sapiens
ATWDSYYKKL

SEQ ID NO: 1323 - protein, 13 aa, Homo sapiens
ALWEVELGKKIKV

SEQ ID NO: 1324 - protein, 14 aa, Homo sapiens
ALWEVSELGKKIKV

SEQ ID NO: 1325 - protein, 14 aa, Homo sapiens
ALWGVQELGKKIKV

SEQ ID NO: 1326 - protein, 9 aa, Homo sapiens
ATWDGYKKL

SEQ ID NO: 1327 - protein, 11 aa, Homo sapiens
ATWDGHYYKKL

SEQ ID NO: 1328 - protein, 14 aa, Homo sapiens
ALWEVQELGKKIRV

SEQ ID NO: 1329 - protein, 14 aa, Homo sapiens
ALWEVQELGKKINV

SEQ ID NO: 1330 - protein, 14 aa, Homo sapiens
ALWEPQELGKKIKV

SEQ ID NO: 1331 - protein, 13 aa, Homo sapiens
ALWEVQLGKKIKV

SEQ ID NO: 1332 - protein, 14 aa, Homo sapiens
ALWEVPELGKKIKV

SEQ ID NO: 1333 - protein, 8 aa, Homo sapiens
ATWDGKKL

SEQ ID NO: 1334 - protein, 11 aa, Homo sapiens
ATWDRPYYKKL

SEQ ID NO: 1335 - protein, 14 aa, Homo sapiens
ALWEVRELGKIIKV

SEQ ID NO: 1336 - protein, 14 aa, Homo sapiens
ALGEVRELGKKIKV

SEQ ID NO: 1337 - protein, 14 aa, Homo sapiens
SLWEVQELGKKIKV

SEQ ID NO: 1338 - protein, 14 aa, Homo sapiens
ALLEVQELGKKIKV

SEQ ID NO: 1339 - protein, 13 aa, Homo sapiens
ALWVQELGKKIKV

SEQ ID NO: 1340 - protein, 14 aa, Homo sapiens
ALWEVVELGKKIKV

SEQ ID NO: 1341 - protein, 10 aa, Homo sapiens
ATWDGHYKKL

SEQ ID NO: 1342 - protein, 11 aa, Homo sapiens
ATWDTTGWFKI

SEQ ID NO: 1343 - protein, 10 aa, Homo sapiens
ATWDGLYKKL

SEQ ID NO: 1344 - protein, 13 aa, Homo sapiens
ALWEVELGKKIKV

SEQ ID NO: 1345 - protein, 14 aa, Homo sapiens
ASWEVQELGKKIKV

SEQ ID NO: 1346 - protein, 14 aa, Homo sapiens
ALWEARELGKKIKV

SEQ ID NO: 1347 - protein, 14 aa, Homo sapiens
AVWEVQELGKKIKV

SEQ ID NO: 1348 - protein, 14 aa, Homo sapiens
ALWEVQDLGKKIKV

SEQ ID NO: 1349 - protein, 10 aa, Homo sapiens
ALWEVYYKKL

SEQ ID NO: 1350 - protein, 10 aa, Homo sapiens
ATWDRYYKKL

SEQ ID NO: 1351 - protein, 11 aa, Homo sapiens
ATWDRRYYKKL

SEQ ID NO: 1352 - protein, 10 aa, Homo sapiens
ATWDNYYKKL

SEQ ID NO: 1353 - protein, 14 aa, Homo sapiens
ALWEVLEFGKKIKV

SEQ ID NO: 1354 - protein, 14 aa, Homo sapiens
ALWEVQELGKKVKV

SEQ ID NO: 1355 - protein, 14 aa, Homo sapiens
ALWEVQELRKKIKV

SEQ ID NO: 1356 - protein, 14 aa, Homo sapiens
ALWEVQELGKKIEV

SEQ ID NO: 1357 - protein, 13 aa, Homo sapiens
ALWEGELGKKIKV

SEQ ID NO: 1358 - protein, 10 aa, Homo sapiens
ATWDRRYKKL

SEQ ID NO: 1359 - protein, 11 aa, Homo sapiens
ATWDGYYYKKL

SEQ ID NO: 1360 - protein, 14 aa, Homo sapiens
ALWEVRELVKKIKV

SEQ ID NO: 1361 - protein, 14 aa, Homo sapiens
ALWEVRELCKKIKV

SEQ ID NO: 1362 - protein, 13 aa, Homo sapiens
ALWEVQEGKKIKV

SEQ ID NO: 1363 - protein, 10 aa, Homo sapiens
ATWENYYKKL

SEQ ID NO: 1364 - protein, 12 aa, Homo sapiens
ATWDGPGYYKKL

SEQ ID NO: 1365 - protein, 11 aa, Homo sapiens
ATWDRLYYKKL

SEQ ID NO: 1366 - protein, 10 aa, Homo sapiens
ATWDVYYKKL

SEQ ID NO: 1367 - protein, 11 aa, Homo sapiens
ATWDGDYYKKL

SEQ ID NO: 1368 - protein, 14 aa, Homo sapiens
ALWEAQEFGKKIKV

SEQ ID NO: 1369 - protein, 14 aa, Homo sapiens
ALWDVRELGKKIKV

SEQ ID NO: 1370 - protein, 11 aa, Homo sapiens
ATWDRPGYKKL

SEQ ID NO: 1371 - protein, 14 aa, Homo sapiens
ALWEGGELGKKIKV

SEQ ID NO: 1372 - protein, 13 aa, Homo sapiens
ALWEKELGKKIKV

(19) United States
(12) Patent Application Publication
(10) Pub. No.: US 2022/0148690 A1
(43) Pub. Date: May 12, 2022

(54) IMMUNOREPERTOIRE WELLNESS ASSESSMENT SYSTEMS AND METHODS

(71) Applicant: iRepertoire, Inc., Huntsville, AL (US)

(72) Inventor: JIAN HAN, (US)

(21) Appl. No.: 17/612,137

(22) PCT Filed: May 18, 2020
(86) PCT No.: PCT/US2020/033451

§ 371 Date: Nov. 17, 2021

Related U.S. Application Data
(60) Provisional application No. 62/849,587, filed on May 17, 2019.

(51) Int. Cl. G16H 10/40; G06K 7/14

(52) U.S. Cl. CPC G16H 10/40; G06K 7/1417

(57) ABSTRACT

The present disclosure relates to systems and methods for assessing the immunorepertoire and wellness of an individual. This disclosure contemplates an individual submitting: (a) identifying information (such as family medical history, age, gender, and other identifying information) to a database on or accessible by a server by connecting a device, such as a smartphone, to a web application; and (b) a blood sample for immune repertoire processing and submission of the resulting data to the database. The data are processed by a server, which accesses a database. The individual may then access a customized report using a web application accessible by a smartphone or other Internet-connected device. The customized report displays the individuals immunorepertoire indexes. Three immunorepertoire indexes disclosed herein include the: (1) clonotype index; (2) essential index; and (3) diversity index. In certain embodiments, the customized report comprises a graphical representation of the individuals immunorepertoire, with the size of a unique clonotype corresponding with the frequency of such clonotype.

### BACKGROUND OF INVENTION

Diagnostic tests are currently available and performed on a regular basis to detect the presence or absence of a normal state in an individual. These tests, however, do not provide a clear assessment of the immunorepertoire in an individual or insight into how such individual's immunorepertoire is indicative of the presence or absence of wellness. A need, therefore, exists for systems and methods to provide individuals with a means of assessing and displaying their immunorepertoire in a manner that may assist with an assessment of the health of such individual.

### SUMMARY OF THE INVENTION

In some embodiments, the present disclosure relates to a method of presenting a user's immunorepertoire profile to the user, comprising the steps of: obtaining a blood sample from the user; determining at least one index for selected from the group consisting of the clonotype index, essential index, and diversity index to produce an immunorepertoire profile for the blood sample for the user; and outputting information to the user pertaining to the user's immunorepertoire profile. In some embodiments, the method further comprises the step of obtaining a set of characteristic data associated with the user, wherein the characteristic data associated with the user comprises the user's age and gender. In some embodiments, the characteristic data further comprises the presence of any disease. In some embodiments, the blood sample comprises whole blood. In some embodiments, the blood sample comprises a dried blood spot. In some embodiments, the, method comprises the additional steps of: providing the user with a kit comprising a blood collection card, wherein the blood collection card comprises at least one blood collection area and a QR code; and scanning the QR code by the user to associate the blood sample with the user's account on a software application. In some embodiments, the step of outputting information to the user is performed using a software application.

In some embodiments, the present disclosure relates to a method of presenting a user's immunorepertoire profile to the user, comprising the steps of: providing the user with a kit comprising a blood collection card, wherein the blood collection card comprises at least one blood collection area and a QR code; scanning the QR code by the user to associate the blood sample with the user's account on a software application; obtaining a set of characteristic data associated with the user, wherein the characteristic data associated with the user comprises the user's age, gender and the presence or absence of any disease; obtaining a blood sample from the user; determining at least one index for selected from the group consisting of the clonotype index, essential index, and diversity index to produce an immunorepertoire profile for the blood sample for the user; and outputting information to the user pertaining to the user's immunorepertoire profile using a software application.

### BRIEF DESCRIPTION OF THE DRAWINGS

FIG. 1 is a flowchart depicting the process by which a user submits: (a) identifying information to a database by connecting a device to a web application; and (b) a blood sample for immune repertoire processing and submission of the resulting data to the database.

FIG. 2 is a flowchart depicting the process by which a user's identifying information and immune repertoire data are processed by a server, referencing a database and incorporating the resulting information into the database, with the resulting clonotype index, diversity index and essential index report made available for display to the user.

FIG. 3 is a flowchart depicting the process by which a user may access their clonotype index, diversity index and essential index report by connecting to a database via a web application using a device.

### DETAILED DESCRIPTION

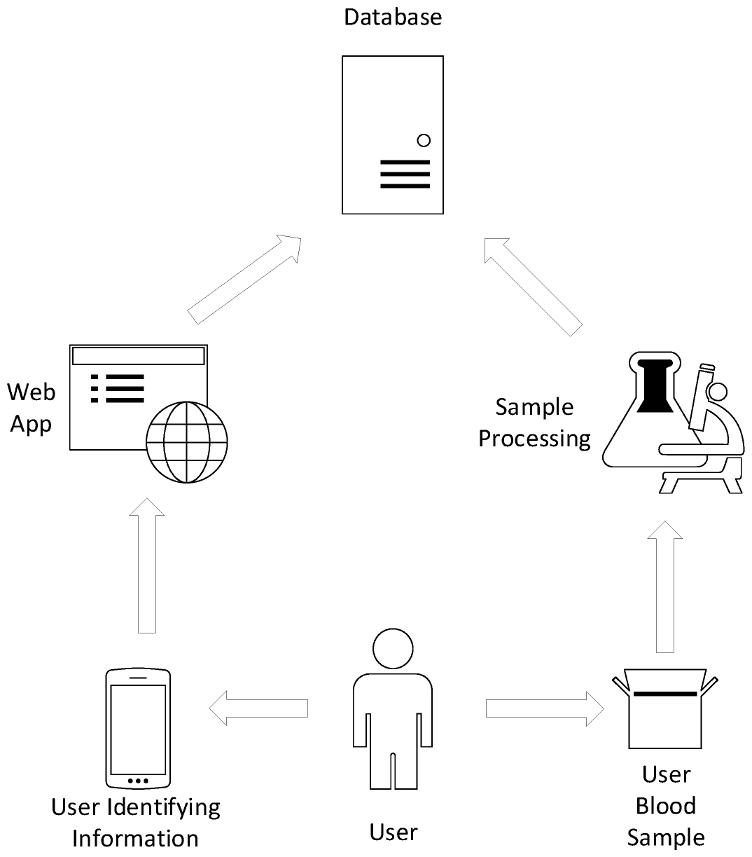

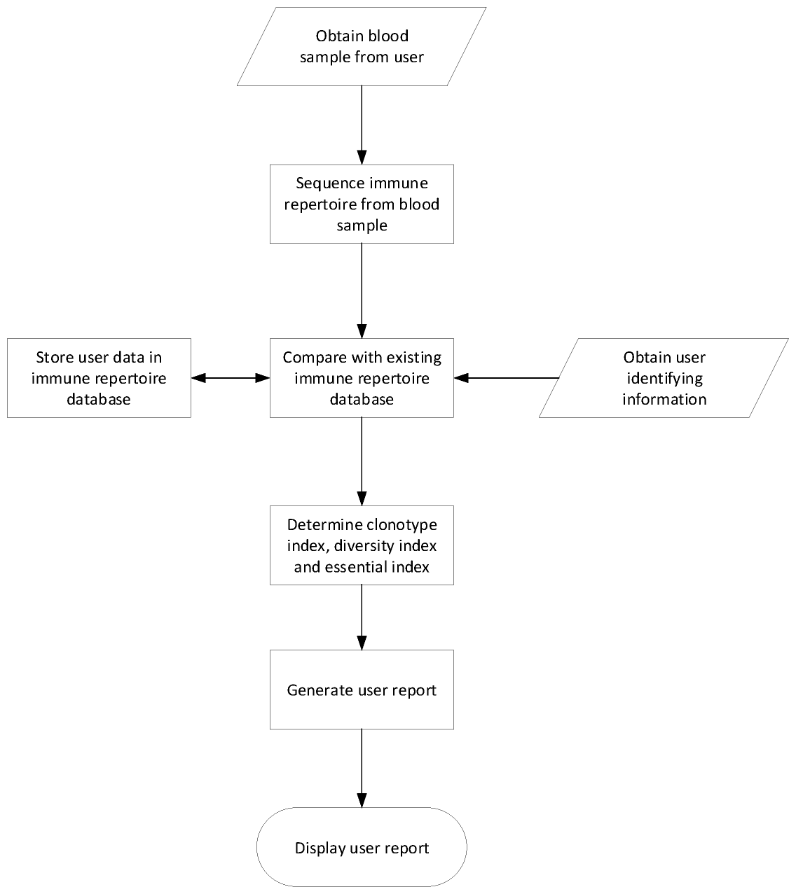

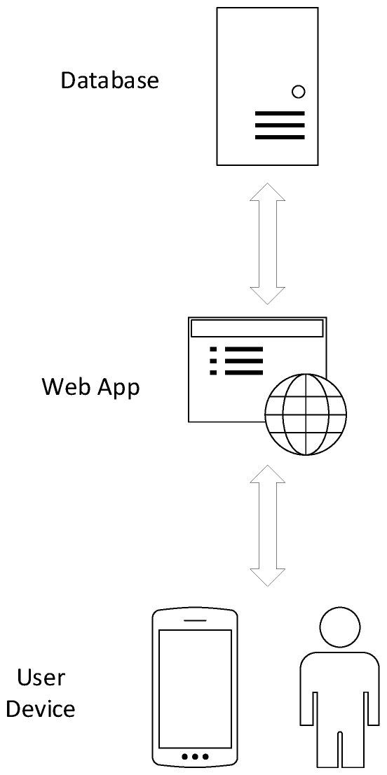

This disclose relates to systems and methods for assessing the immunorepertoire and wellness of an individual. As depicted in FIG. 1, this disclosure contemplates an individual submitting: (a) identifying information (such as family medical history, age, gender, and other identifying information) to a database on or accessible by a server by connecting a device, such as a smartphone, to a web application; and (b) a blood sample for immune repertoire processing and submission of the resulting data to the database. The data are processed by a server, which accesses the database, as depicted in FIG. 2, to create a custom report for the user. The individual may then access a customized report using a web application accessible by a smartphone or other Internet-connected device, as depicted in FIG. 3. The customized report displays the individual's immunorepertoire indexes. Three immunorepertoire indexes disclosed herein include the: (1) clonotype index; (2) essential index; and (3) diversity index. In certain embodiments, the customized report comprises a graphical representation of the individual's immunorepertoire, with the size of a unique clonotype corresponding with the frequency of such clonotype.

In some embodiments, the blood sample may be collected by a user by using a kit comprising a lancet and a sterile blood collection card. The blood collection card may comprise materials suitable for absorbing blood, including but not limited to paper and card stock. A user may use the lancet to draw blood, for example from one of the user's fingertips. The blood collection card comprises one or more blood collection areas on which the user may place a sample of blood and where such blood may dry. The blood collection card may further comprise a QR code, which the user may scan using a smartphone or other device to associate the QR code and the blood sample with the user's account on a software application. The user may then send the blood collection card for rehydration, processing and determination of the user's clonotype index, essential index, and/or diversity index to generate a user report which is stored on a database. The user may then access his or her user report stored on the database using the software application via an internet connected device.

### Clonotype Index

The first index disclosed herein is referred to as the clonotype index. The clonotype index for an individual is obtained by measuring the total number of unique clonotypes in an individual's sample containing lymphocytes, such as a blood sample, and dividing the number of unique clonotypes by the number of unit reads for such sample. As used herein, “blood sample” means peripheral blood, a dried blood spot, cord blood, or other sample containing blood.

### Essential Index

The second index disclosed herein is referred to as the essential index. In one embodiment, the essential index is the number of the top 1000 public CDR3s (pCDR3s) in 100,000 of an individual's reads. pCDR3s are CDR3s present in more than one individual. For purposes of determining the top 1000 pCDR3s, the pCDR3s of a cohort of individuals (index pool) is determined and ranked. In other embodiments, fewer than the top 1000 pCDRs are assessed. In other embodiments, more than the top 1000 pCDR3s are assessed. In other embodiments, fewer than the 100,000 reads are taken for an individual. In other embodiments, more than the 100,000 reads are taken for an individual.

In one aspect of the present disclosure, the immunorepertoire of an individual is considered normal if the individual's essential index meets or exceeds a minimum percentage, whereas the immunorepertoire of the individual is considered abnormal of the individual's normality index is below such minimum percentage. In one embodiment, the minimum percentage is 35%.

The CDR3 expressed by individuals exhibits tremendous diversity, with up to 10¹⁵ unique CDR3 possible. As such, CDR3 may be used as a basis for immune system diversity. Based on a sampling of 75 million CDR3, the inventor has determined that approximately 81% of randomly-selected CDR3 are unique to a given individual and are not shared among multiple individuals.

The method of the present disclosure may be performed using the following steps to identify a normal immune status or an abnormal immune status in an individual, the method comprising the steps of: (a) amplifying polynucleotides from a population of white blood cells from an individual in a reaction mix comprising target-specific nested primers to produce a set of first amplicons, at least a portion of the target-specific nested primers comprising additional nucleotides which, during amplification, serve as a template for incorporating into the first amplicons a binding site for at least one common primer; (b) transferring a portion of the first reaction mix containing the first amplicons to a second reaction mix comprising at least one common primer; (c) amplifying, using the at least one common primer, the first amplicons to produce a set of second amplicons; (d) sequencing the second amplicons to identify CDR3 sequences in the subpopulation of white blood cells, and (e) identifying CDR3 sequences which constitute pCDR3s; (f) calculating the essential index based on the individual's pCDR3s; and (g) identifying whether the essential index is normal or abnormal, wherein a normal state is characterized by the presence of a minimum percentage of pCDR3 and an abnormal state is characterized by the absence of a minimum percentage of pCDR3.

In certain embodiments, the sequencing includes about 100,000 reads taken per sample. In certain embodiments, the reads are performed multiple times, for example about 10 to 100 times, using random selection. The number of an individual's pCDR3 in the top 1000 pCDR3s of the reference pool provide a percentage, referred to as the “essential index,” which is a number between 0% and 100%. For example, if an individual's sample contains 200 of the top 1000 pCDR3 sequences, then the individual's essential index is 0.20 or 20%. In other embodiments at least 10,000 reads are taken. In other embodiments, more than 100,000 reads are taken. In other embodiments, the reads are performed less than 10 times. In other embodiments, the reads are performed more than 100 times.

In certain embodiments, the index pool is composed of about 1000 individuals. In other embodiments, the index pool contains between 100 and 1000 individuals. In other embodiments, the index pool contains fewer than 100 individuals. In other embodiments, the index pool contains more than 1000 individuals. Relative to the individual, the individuals may be age-matched, gender-matched, healthy, disease-matched, and/or other criteria commonly known in the art when controlling for variables. In certain embodiments, the index pool is composed of healthy controls. In other embodiments, the index pool is composed of a mix of healthy controls and individuals with one or more disease states. In other embodiments, the index pool is composed of individuals with one or more particular disease states.

In certain embodiments, the CDR3 sequences shared by the index pool (i.e., the pCDR3) are determined by comparing each sample from the index pool and identifying those CDR3s that are shared by at least 50% of the individuals tested in such reference pool. In certain embodiments, the pCDR3 includes about the top 1000 shared CDR3 sequences. In other embodiments, the pCDR3 include at least 100 CDR3 sequences. In other embodiments, the pCDR3 includes more than 1000 CDR3 sequences.

It has previously been difficult to assess the immune system in a broad manner, because the number and variety of cells in a human or animal immune system is so large that sequencing of more than a small subset of cells has been almost impossible. The inventor developed a semi-quantitative PCR method (arm-PCR, described in more detail in U.S. Patent Application Publication Number 20090253183), which provides increased sensitivity and specificity over previously-available methods, while producing semi-quantitative results. It is this ability to increase specificity and sensitivity, and thereby increase the number of targets detectable within a single sample that makes the method ideal for detecting relative numbers of clonotypes of the immunorepertoire. The inventor has more recently discovered that using this sequencing method allows comparison of an individual's CDR3 sequences to those commonly shared by an index group, which has led to the development of the present method. The method may be used to evaluate the diversity of the immunorepertoire of subjects relative to an index pool of individuals. For example, the inventor has demonstrated that the presence of disease correlates with decreased immunorepertoire diversity, for example a decrease in the diversity of CDR3 sequences, which can be readily detected using the method of the present disclosure. This method may therefore be useful as an initial diagnostic indicator, much as cell counts and biochemical tests are currently used in clinical practice, of normal versus abnormal immunorepertoire diversity.

Clonotypes (i.e., clonal types) of an immunorepertoire are determined by the rearrangement of Variable(V), Diverse(D) and Joining(J) gene segments through somatic recombination in the early stages of immunoglobulin (Ig) and T cell receptor (TCR) production of the immune system. The V(D)J rearrangement can be amplified and detected from T cell receptor alpha, beta, gamma, and delta chains, as well as from immunoglobulin heavy chain (IgH) and light chains (IgK, IgL). Cells may be obtained from an individual by obtaining peripheral blood, lymphoid tissue, cancer tissue, or tissue or fluids from other organs and/or organ systems, for example. Techniques for obtaining these samples, such as blood samples, are known to those of skill in the art. Cell counts may be extrapolated from the number of sequences detected by PCR amplification and sequencing.

The CDR3 region, comprising about 30-90 nucleotides, encompasses the junction of the recombined variable (V), diversity (D) and joining (J) segments of the gene. It encodes the binding specificity of the receptor and is useful as a sequence tag to identify unique V(D)J rearrangements.

Wang et al. disclosed that PCR may be used to obtain quantitative or semi-quantitative assessments of the numbers of target molecules in a specimen (Wang, M. et al, “Quantitation of mRNA by the polymerase chain reaction,” (1989) Proc. Nat'l. Acad. Sci. 86: 9717-9721). Particularly effective methods for achieving quantitative amplification have been described previously by the inventor. One such method is known as arm-PCR, which is described in United States Patent Application Publication Number 20090253183A1.

Aspects of the present disclosure include arm-PCR amplification of CDR3 from T cells, B cells, and/or subsets of T or B cells. Such cell types may be sorted and isolated using techniques known in the art including, but not limited to, FACS sorting and magnetic bead sorting. The term “population” of cells, as used herein, therefore encompasses what are generally referred to as either “populations” or “sub-populations” of cells. Large numbers of amplified products may then be efficiently sequenced using next-generation sequencing using platforms such as 454 or Illumina, for example.

The arm-PCR method provides highly sensitive, semi-quantitative amplification of multiple polynucleotides in one reaction. The arm-PCR method may also be performed by automated methods in a closed cassette system (iCubate®, Huntsville, Ala.), which is beneficial in the present method because the repertoires of various T and B cells, for example, are so large. In the arm-PCR method, target numbers are increased in a reaction driven by DNA polymerase, which is the result of target-specific primers being introduced into the reaction. An additional result of this amplification reaction is the introduction of binding sites for common primers which will be used in a subsequent amplification by transferring a portion of the first reaction mix containing the first set of amplicons to a second reaction mix comprising common primers. “At least one common primer,” as used herein, refers to at least one primer that will bind to such a binding site, and includes pairs of primers, such as forward and reverse primers. This transfer may be performed either by recovering a portion of the reaction mix from the first amplification reaction and introducing that sample into a second reaction tube or chamber, or by removing a portion of the liquid from the completed first amplification, leaving behind a portion, and adding fresh reagents into the tube in which the first amplification was performed. In either case, additional buffers, polymerase, etc., may then be added in conjunction with the common primers to produce amplified products for detection. The amplification of target molecules using common primers gives a semi-quantitative result wherein the quantitative numbers of targets amplified in the first amplification are amplified using common, rather than target-specific primers—making it possible to produce significantly higher numbers of targets for detection and to determine the relative amounts of the cells comprising various rearrangements within an individual blood sample. Also, combining the second reaction mix with a portion of the first reaction mix allows for higher concentrations of target-specific primers to be added to the first reaction mix, resulting in greater sensitivity in the first amplification reaction. It is the combination of specificity and sensitivity, along with the ability to achieve quantitative results by use of a method such as the arm-PCR method, which allows a sufficiently sensitive and quantitative assessment of the CDR3 expressed in a population of cells to produce a normality index that is of diagnostic use.

Clonal expansion due to recognition of antigen results in a larger population of cells that recognize that antigen, potentially including antibody-producing B cells or receptor-bearing T cells. This may cause the reads taken pursuant to the method disclosed herein to be biased in favor of the antigen-specific expansion, thereby reducing the percentage of pCDR3 sequences detected. Therefore, a relatively low normality index, for example one below the minimum percentage, may be indicative of the expansion of a particular population of cells that is prevalent in individuals who have been diagnosed with a particular disease or in individuals recently-vaccinated against a particular antigen.

Primers for amplifying and sequencing variable regions of immune system cells are available commercially, and have been described in publication such as the inventor's published patent applications WO2009137255 and US201000021896A1.

There are several commercially available high-throughput sequencing technologies, such as Hoffman-LaRoche, Inc.'s 454® sequencing system. In the 454® sequencing method, for example, the A and B adaptor are linked onto PCR products either during PCR or ligated on after the PCR reaction. The adaptors are used for amplification and sequencing steps. When done in conjunction with the arm-PCR technique, A and B adaptors may be used as common primers (which are sometimes referred to as “communal primers” or “superprimers”) in the amplification reactions. After A and B adaptors have been physically attached to a sample library (such as PCR amplicons), a single-stranded DNA library is prepared using techniques known to those of skill in the art. The single-stranded DNA library is immobilized onto specifically-designed DNA capture beads. Each bead carries a unique singled-stranded DNA library fragment. The bead-bound library is emulsified with amplification reagents in a water-in-oil mixture, producing microreactors, each containing just one bead with one unique sample-library fragment. Each unique sample library fragment is amplified within its own microreactor, excluding competing or contaminating sequences. Amplification of the entire fragment collection is done in parallel. For each fragment, this results in copy numbers of several million per bead. Subsequently, the emulsion PCR is broken while the amplified fragments remain bound to their specific beads. The clonally amplified fragments are enriched and loaded onto a PicoTiterPlate® device for sequencing. The diameter of the PicoTiterPlate® wells allows for only one bead per well. After addition of sequencing enzymes, the fluidics subsystem of the sequencing instrument flows individual nucleotides in a fixed order across the hundreds of thousands of wells each containing a single bead. Addition of one (or more) nucleotide(s) complementary to the template strand results in a chemilluminescent signal recorded by a CCD camera within the instrument. The combination of signal intensity and positional information generated across the PicoTiterPlate® device allows the software to determine the sequence of more than 1,000,000 individual reads, each is up to about 450 base pairs, with the GS FLX system.

Having obtained the sequences using a quantitative and/or semi-quantitative method, it is then possible to calculate the normality index, for example, by determining the percentage of pCDR3 represented by a predetermined number of reads of an individual sample. Each individual's normality index may be compared to a predetermined threshold to determine whether the individual's normality index falls within the normal range, and therefore is normal, or below the threshold, and thereby is abnormal.

The method of the present disclosure provides a physician with an additional clinical test for diagnostic purposes to determine whether an individual's immunorepertoire is abnormal. Further, the method of the present disclosure, particularly if used in an automated system such as that described by the inventor in U.S. Patent Application Publication Number 201000291668A1, may be used to analyze samples from multiple individuals, with detection of the amplified targets sequences being accomplished by the use of one or more microarrays.

### Examples

### Individual Samples

Whole blood samples (40 ml) collected in sodium heparin or peripheral blood mononuclear cells (PBMCs) were obtained from 1100 individuals, representing a mixed population of both healthy individuals and those with disease. The 1100 individuals were placed randomly into 11 different groups with 100 samples per group.

### RNA Extraction and Repertoire Amplification

RNA extraction was performed using the RNeasy Mini Kit (Qiagen) according to the manufacturer's protocol. For each target, a set of nested sequence-specific primers (Forward-out, Fo; Forward-in, Fi; Reverse-out, Ro; and Reverse-in, Ri) was designed using primer software available at www.irepertoire.com. A pair of common sequence tags was linked to all internal primers (Fi and Ri). Once these tag sequences were incorporated into the PCR products in the first few amplification cycles, the exponential phase of the amplification was carried out with a pair of communal primers. In the first round of amplification, only sequence-specific nested primers were used. The nested primers were then removed by exonuclease digestion and the first-round PCR products were used as templates for a second round of amplification by adding communal primers and a mixture of fresh enzyme and dNTP. Each distinct barcode tag was introduced into amplicon from the same sample through PCR primer.

### Sequencing

Barcode tagged amplicon products from different samples were pooled together and loaded into a 2% agarose gel. Following electrophoresis, DNA fragments were purified from DNA band corresponding to 250-500 bp fragments extracted from agarose gel. DNA was sequenced using the 454 GS FLX system with titanium kits (SeqWright, Inc.).

### Sequencing Data Analysis

Sequences for each sample were sorted out according to barcode tag. Following sequence separation, sequence analysis was performed in a manner similar to the approach reported by Wang et al. (Wang C, et al. High throughput sequencing reveals a complex pattern of dynamic interrelationships among human T cell subsets. Proc Natl Acad Sci USA 107(4): 1518-1523). Briefly, germline V and J reference sequences, which were downloaded from the IMGT server (http://www.imgt.org), were mapped onto sequence reads using the program IRmap. The boundaries defining CDR3 region in reference sequences were mirrored onto sequencing reads through mapping information. The enclosed CDR3 regions in sequencing reads were extracted and translated into amino acid sequence.

Table 1 below lists exemplary pCDR3 from cord blood. Table 2 below lists exemplary pCDR3 from adult blood.

TABLE 1
IgH:

| | | | Adults | Adult | |
|---|---|---|---|---|---|
| | CB with | CB total | with | total | Adult |
| CDR3 | CDR3 | reads | CDR3 | reads | rank |
| * | 30 | 10026640 | 222 | 5712880 | 0 |
| ARDSSSWYYFDY | 30 | 57228 | 36 | 526 | 51 |
| ARDSSGWYYFDY | 30 | 45778 | 67 | 1697 | 5 |
| ARDAFDI | 30 | 32844 | 51 | 1157 | 18 |
| ARDSSSFDY | 30 | 30408 | 9 | 141 | 1483 |
| ARGYCSSTSCYDAFDI | 30 | 28254 | 10 | 119 | 1267 |
| ARGYSSSWYFDY | 30 | 26430 | 17 | 469 | 327 |
| AREYSSSFDY | 30 | 22984 | 15 | 265 | 475 |
| ARVGYSSSWYYFDY | 30 | 20807 | 12 | 223 | 786 |
| ARGLDY | 30 | 18643 | 60 | 1756 | 10 |
| ARGDAFDI | 30 | 16700 | 46 | 1027 | 20 |
| ARGYSSSWYDY | 30 | 16486 | 14 | 242 | 544 |
| ARGSSSFDY | 30 | 16382 | 13 | 269 | 629 |
| ARGYCSGGSCYYFDY | 30 | 16346 | 19 | 394 | 254 |
| ARGDY | 30 | 15855 | 53 | 1292 | 15 |
| ARGSGSYFDY | 30 | 15592 | 14 | 241 | 545 |
| ARVYSSSWYYFDY | 30 | 14856 | 22 | 380 | 177 |
| ARGVDY | 30 | 10891 | 44 | 597 | 24 |
| ARDPDY | 30 | 10676 | 66 | 1426 | 8 |
| ARGYSGYDFDY | 30 | 8668 | 9 | 60 | 1664 |
| ARDSSSWYFDY | 29 | 49116 | 19 | 250 | 273 |
| ARDSSSSFDY | 29 | 48306 | 21 | 452 | 192 |
| ARGYSSSWYYFDY | 29 | 46153 | 46 | 944 | 21 |
| ARDLDY | 29 | 42355 | 124 | 4569 | 1 |
| ARDSGSYYFDY | 29 | 35688 | 33 | 578 | 64 |
| ARYSSSWYYFDY | 29 | 29284 | 27 | 466 | 100 |
| ARDSGSYFDY | 29 | 22525 | 16 | 229 | 410 |
| ARGYSSSWYWFDP | 29 | 16637 | 20 | 314 | 231 |
| ARGGSYFDY | 29 | 16227 | 17 | 196 | 356 |
| ARDHSSSWYYFDY | 29 | 15470 | 10 | 169 | 1187 |
| ARAYSSSWYYFDY | 29 | 15437 | 24 | 827 | 130 |
| ARGFDY | 29 | 15010 | 69 | 1410 | 4 |
| ARGYSSGWYYFDY | 29 | 13979 | 42 | 778 | 29 |
| ARDSSSWYAFDI | 29 | 12573 | | | |
| ARGYSSSWYAFDI | 29 | 11493 | | | |
| ARDSSSWYYYYYGMDV | 29 | 11166 | 14 | 451 | 521 |
| ARGYSSGWYDY | 29 | 11077 | 17 | 406 | 331 |
| ARGGSSWYYFDY | 29 | 10975 | 14 | 238 | 546 |
| ARGGYYFDY | 29 | 10059 | 40 | 689 | 35 |
| ARGDSSSWYYFDY | 29 | 9658 | 19 | 200 | 277 |
| ARGGYSSSWYYFDY | 29 | 9543 | 21 | 296 | 204 |
| ARVYSSSWYFDY | 29 | 9032 | 2 | 14 | 90579 |
| ARDRGSYYFDY | 29 | 8794 | 15 | 366 | 452 |
| ARVSSSWYYFDY | 29 | 8369 | 20 | 280 | 236 |
| ARDSGSYYYYYGMDV | 29 | 8303 | 7 | 77 | 2773 |
| ARDGGSYFDY | 29 | 7555 | 13 | 151 | 691 |
| ARYYYDSSGYYYFDY | 29 | 7540 | 23 | 337 | 159 |
| ARGSSGWYYFDY | 29 | 7516 | 34 | 367 | 60 |
| ARGYYDSSGYYYFDY | 29 | 7279 | 30 | 450 | 77 |
| ARGDYYFDY | 29 | 6895 | 26 | 482 | 107 |
| ARGSSSWYDY | 29 | 6595 | 4 | 43 | 9913 |
| ARDRSGSYFDY | 29 | 6129 | 8 | 124 | 1919 |
| ARVYSSGWYYFDY | 29 | 5245 | 21 | 306 | 201 |
| TTLDY | 29 | 3812 | 12 | 207 | 794 |
| ARDSGSWYYFDY | 29 | 795 | 2 | 2 | 127432 |
| ARDCSSTSCYDY | 28 | 37873 | 6 | 110 | 3441 |
| ARDFDY | 28 | 25373 | 103 | 3898 | 2 |
| ARDDY | 28 | 21352 | 72 | 2383 | 3 |
| ARDDAFDI | 28 | 20888 | 44 | 1082 | 23 |
| AKDSSSWYYFDY | 28 | 20174 | 13 | 143 | 696 |
| ARDCSGGSCYFDY | 28 | 19329 | 9 | 301 | 1360 |
| ARDSSSYYFDY | 28 | 18735 | 4 | 51 | 9439 |
| ARGSSSWYYFDY | 28 | 18642 | 27 | 449 | 101 |
| ARYSSSWYFDY | 28 | 17220 | 12 | 171 | 825 |
| ARGYSSSWFDY | 28 | 15163 | 10 | 142 | 1235 |
| ARGYCSGGSCYFDY | 28 | 14989 | 28 | 479 | 88 |
| AKDSSSWYFDY | 28 | 14415 | 5 | 85 | 5237 |
| ARDCSSTSCFDY | 28 | 14167 | 8 | 110 | 1977 |
| ARGSGSYYFDY | 28 | 13865 | 13 | 304 | 619 |
| AREDY | 28 | 13371 | 67 | 1612 | 6 |
| ARDLGY | 28 | 13309 | 64 | 1334 | 9 |
| ARDYYDSSGYYYFDY | 28 | 12647 | 39 | 705 | 39 |
| ARVDY | 28 | 12619 | 56 | 1206 | 13 |
| ARGYCSSTSCYDY | 28 | 12371 | 7 | 125 | 2481 |
| ARSYSSSWYYFDY | 28 | 11675 | 22 | 239 | 181 |
| ARGSSSWYFDY | 28 | 11539 | 5 | 69 | 5556 |
| TTVDY | 28 | 11285 | 28 | 780 | 84 |
| ARDSGSYYYFDY | 28 | 11098 | 3 | 33 | 20173 |
| ARDRGSYFDY | 28 | 10779 | 16 | 487 | 372 |
| ARGDYFDY | 28 | 10435 | 23 | 551 | 151 |
| ARGYCSSTSCYFDY | 28 | 10252 | 13 | 155 | 688 |
| ARDRDAFDI | 28 | 9986 | 29 | 481 | 83 |
| ARAYSSSWYFDY | 28 | 9879 | 7 | 93 | 2667 |
| ARGGYSSSWYWFDP | 28 | 9361 | 4 | 63 | 8795 |
| ARDYDY | 28 | 9013 | 29 | 698 | 79 |
| ARDLDAFDI | 28 | 8944 | 22 | 305 | 179 |
| AREGY | 28 | 8903 | 39 | 1036 | 36 |
| ARGSGSYYYFDY | 28 | 7808 | 6 | 112 | 3422 |
| ARGSGSYYYYYGMDV | 28 | 7742 | 10 | 83 | 1306 |
| ARELDY | 28 | 7647 | 37 | 806 | 45 |
| ARGGDYFDY | 28 | 7419 | 19 | 572 | 248 |
| ARGYSSSYYFDY | 28 | 7257 | 7 | 129 | 2458 |
| ARGVGATDY | 28 | 6490 | 10 | 192 | 1153 |
| ARDGAFDI | 28 | 6441 | 20 | 288 | 235 |
| ARRGYSSSWYYFDY | 28 | 6258 | 13 | 176 | 673 |
| ARGADY | 28 | 6218 | 17 | 287 | 340 |
| ARGYSSSWYNWFDP | 28 | 6091 | 8 | 173 | 1797 |
| ARDSSSWYYYYGMDV | 28 | 6031 | 11 | 69 | 1062 |
| ARVYSSSFDY | 28 | 5592 | 3 | 40 | 18740 |

IgK:

| | | | Adults | Adult | |
|---|---|---|---|---|---|
| | CB with | CB total | with | total | Adult |
| CDR3 | CDR3 | reads | CDR3 | reads | rank |
| * | 12 | 6170981 | 222 | 14114525 | 0 |
| QQSYSTPYT | 12 | 3597815 | 222 | 2163695 | 6 |
| QQYDNLPLT | 12 | 2066846 | 222 | 3317473 | 1 |
| QQSYSTPRT | 12 | 1807334 | 222 | 2952854 | 3 |
| QQYGSSPRT | 12 | 1762387 | 222 | 3303511 | 2 |
| QQYGSSPYT | 12 | 1694095 | 222 | 1815657 | 9 |
| QQSYSTPWT | 12 | 1571128 | 222 | 1695770 | 10 |
| QQYGSSPWT | 12 | 1441308 | 222 | 2609412 | 5 |
| QQYDNLPYT | 12 | 1267862 | 222 | 1296252 | 15 |
| QQSYSTPFT | 12 | 1186065 | 222 | 714272 | 42 |
| QQYGSSPLT | 12 | 1028477 | 222 | 1867304 | 8 |
| QQSYSTPLT | 12 | 966727 | 222 | 1303006 | 14 |
| LQHNSYPWT | 12 | 944829 | 222 | 866476 | 27 |
| QQYGSSPT | 12 | 847464 | 222 | 573641 | 49 |
| QQSYSTPPT | 12 | 782033 | 222 | 963899 | 23 |
| QQYNNWPPWT | 12 | 760699 | 222 | 2005449 | 7 |
| QQYGSSPPYT | 12 | 691757 | 222 | 972444 | 22 |
| QQYNNWPPYT | 12 | 680244 | 222 | 1272554 | 16 |
| QQYYSYPRT | 12 | 664430 | 222 | 1067391 | 20 |
| QQYNNWPRT | 12 | 663030 | 222 | 1672121 | 11 |
| QQSYSTPIT | 12 | 644068 | 222 | 499000 | 62 |
| LQHNSYPYT | 12 | 643754 | 222 | 394879 | 77 |
| QQYDNLPIT | 12 | 635718 | 222 | 850500 | 30 |
| QQYGSSPFT | 12 | 612647 | 222 | 855515 | 29 |
| QQYGSSPIT | 12 | 556768 | 222 | 832529 | 33 |
| QQYNNWPPLT | 12 | 548699 | 222 | 1203855 | 17 |
| QQYDNLPFT | 12 | 548132 | 222 | 640004 | 46 |
| QQRSNWPLT | 12 | 547580 | 222 | 1578220 | 12 |
| LQHNSYPRT | 12 | 544829 | 222 | 782633 | 35 |
| QQYNSYST | 12 | 523209 | 222 | 405406 | 76 |
| QQYNSYWT | 12 | 499060 | 222 | 737411 | 38 |
| QQYYSTPYT | 12 | 495875 | 222 | 2681529 | 4 |
| QQYGSSPPWT | 12 | 487256 | 222 | 752147 | 37 |
| QQYNSYPYT | 12 | 485682 | 222 | 1129544 | 18 |
| QQYNNWPLT | 12 | 480104 | 222 | 1031985 | 21 |
| QQYYSYPYT | 12 | 465884 | 222 | 333528 | 87 |
| QQYNSYSWT | 12 | 453212 | 222 | 876221 | 26 |
| QQYNSYPLT | 12 | 447639 | 222 | 1068076 | 19 |
| QQYYSTPLT | 12 | 433426 | 222 | 862723 | 28 |
| QQRSNWPPIT | 12 | 426878 | 222 | 896207 | 24 |
| QQYNNWPYT | 12 | 398122 | 222 | 514100 | 60 |
| QQYYSYPWT | 12 | 393570 | 222 | 445511 | 69 |
| QQYNSYSRT | 12 | 391904 | 222 | 836163 | 32 |
| LQHNSYPLT | 12 | 382836 | 222 | 564345 | 51 |
| QQRSNWPPYT | 12 | 370863 | 222 | 753751 | 36 |
| QQYNSYSYT | 12 | 370287 | 222 | 471334 | 66 |
| QQYYSYPLT | 12 | 361244 | 222 | 525580 | 57 |
| QQRSNWPPLT | 12 | 356112 | 222 | 815084 | 34 |
| QQYGSSRT | 12 | 355708 | 222 | 430612 | 71 |
| QQYGSSPPT | 12 | 330406 | 222 | 540517 | 54 |
| QQYGSSPQT | 12 | 321515 | 222 | 726922 | 39 |
| QQYGSSPPIT | 12 | 316377 | 222 | 514147 | 59 |
| QQYNSYPWT | 12 | 315545 | 222 | 1438088 | 13 |
| QQYYSTPWT | 12 | 313675 | 222 | 482125 | 64 |
| QQRSNWPPT | 12 | 312412 | 222 | 893836 | 25 |
| QQLNSYPLT | 12 | 301627 | 222 | 718912 | 41 |
| QQYYSYPFT | 12 | 297741 | 222 | 214945 | 131 |
| MQALQTPYT | 12 | 278214 | 222 | 525224 | 58 |
| QQYDNLPPT | 12 | 263221 | 222 | 679053 | 43 |
| QQYNNWPPIT | 12 | 258796 | 222 | 674010 | 44 |
| QQSYSTPPYT | 12 | 254243 | 222 | 343511 | 83 |
| QQYGSSPMYT | 12 | 249635 | 222 | 257209 | 109 |
| QQYGSSLT | 12 | 245653 | 222 | 278642 | 101 |
| QQYGSSPPLT | 12 | 245266 | 222 | 419721 | 74 |
| QQSYSTPHT | 12 | 231726 | 222 | 279756 | 100 |
| QQRSNWPIT | 12 | 230867 | 222 | 546612 | 52 |
| QQYGSSPGT | 12 | 228566 | 222 | 719365 | 40 |
| QQRSNWPPWT | 12 | 225414 | 222 | 659585 | 45 |
| QQYGSSLWT | 12 | 224617 | 222 | 597010 | 48 |
| QQRSNWPRT | 12 | 221567 | 222 | 842959 | 31 |
| QQYDNLPRT | 12 | 220911 | 222 | 536453 | 55 |
| QQSYSTPT | 12 | 217458 | 222 | 230933 | 121 |
| QQYDNLPPLT | 12 | 212553 | 222 | 288444 | 96 |
| QQSYSTPQT | 12 | 211802 | 222 | 475924 | 65 |
| MQALQTPLT | 12 | 210576 | 222 | 543098 | 53 |
| QQYGSSLYT | 12 | 209150 | 222 | 340898 | 84 |
| QQRSNWPT | 12 | 205730 | 222 | 347410 | 81 |
| QQYGSSYT | 12 | 204769 | 222 | 157581 | 160 |
| QQYDNLPPYT | 12 | 203318 | 222 | 346088 | 82 |
| QQYYSTPRT | 12 | 202753 | 222 | 535684 | 56 |
| QQSYSTPYS | 12 | 197767 | 222 | 73774 | 276 |
| QQSYSTPCS | 12 | 197090 | 88 | 55434 | 14335 |
| QQSYSTPPWT | 12 | 196715 | 222 | 280381 | 99 |
| QQANSFPLT | 12 | 193699 | 222 | 498947 | 63 |
| QQLNSYPFT | 12 | 193197 | 222 | 280897 | 98 |
| QQLNSYPYT | 12 | 187840 | 222 | 221395 | 126 |
| QQYYSFPYT | 12 | 186828 | 222 | 102339 | 210 |
| MQALQTPWT | 12 | 186378 | 222 | 513198 | 61 |
| QQYGSSFT | 12 | 183276 | 222 | 146862 | 171 |
| QQLNSYPRT | 12 | 182332 | 222 | 453795 | 67 |
| QQSYSTLWT | 12 | 179915 | 222 | 431092 | 70 |
| LQHNSYPPT | 12 | 178978 | 222 | 264619 | 106 |
| QQRSNWPPFT | 12 | 176371 | 222 | 300165 | 92 |
| QQRSNWYT | 12 | 176056 | 221 | 159336 | 625 |
| QQYNSYPIT | 12 | 174782 | 222 | 281622 | 97 |
| QQYNSYPFT | 12 | 173418 | 222 | 314885 | 88 |
| QQYYSTPPT | 12 | 172158 | 222 | 412231 | 75 |
| QQYYSTPFT | 12 | 171804 | 222 | 238699 | 118 |
| MQALQTPRT | 12 | 171478 | 222 | 635277 | 47 |

IgL:

| | | | Adults | Adult | |
|---|---|---|---|---|---|
| | CB with | CB total | with | total | Adult |
| CDR3 | CDR3 | reads | CDR3 | reads | rank |
| * | 12 | 3459321 | 222 | 8615976 | 0 |
| GTWDSSLSAVV | 12 | 2953276 | 222 | 2542422 | 2 |
| SSYTSSSTLV | 12 | 2920518 | 222 | 921747 | 4 |
| GTWDSSLSAGV | 12 | 2233556 | 222 | 2656155 | 1 |
| SSYTSSSTVV | 12 | 1762737 | 222 | 460353 | 10 |
| QSYDSSLSGSV | 12 | 1355919 | 222 | 865170 | 5 |
| SSYTSSSTWV | 12 | 1296277 | 222 | 358782 | 13 |
| SSYTSSSTLVV | 12 | 1153503 | 222 | 477560 | 9 |
| QVWDSSSDHVV | 12 | 1140242 | 222 | 1009126 | 3 |
| NSRDSSGNHLV | 12 | 938981 | 222 | 449929 | 11 |
| QSADSSGTYVV | 12 | 874443 | 222 | 761696 | 6 |
| SSYTSSSTYV | 12 | 853080 | 221 | 118239 | 103 |
| SSYAGSNNLV | 12 | 762549 | 222 | 300511 | 19 |
| NSRDSSGNHVV | 12 | 710309 | 222 | 319786 | 17 |
| QSYDSSLSGWV | 12 | 594366 | 222 | 566201 | 8 |
| AAWDDSLNGPV | 12 | 580611 | 222 | 422893 | 12 |
| QSYDSSLSGYV | 12 | 555044 | 222 | 230769 | 23 |
| NSRDSSGNHWV | 12 | 520365 | 222 | 348395 | 15 |
| CSYAGSSTLV | 12 | 478979 | 222 | 245335 | 22 |
| GTWDSSLSAWV | 12 | 478468 | 222 | 692370 | 7 |
| AAWDDSLNGWV | 12 | 460547 | 221 | 434855 | 97 |
| QVWDSSSDHPV | 12 | 421063 | 222 | 247647 | 21 |
| SSYTSSSTRV | 12 | 419596 | 222 | 301059 | 18 |
| QVWDSSSDHWV | 12 | 398316 | 221 | 463184 | 96 |
| QSYDSSLSGVV | 12 | 394146 | 222 | 122678 | 38 |
| GTWDSSLSAYV | 12 | 386293 | 222 | 122965 | 37 |
| QAWDSSTVV | 12 | 321889 | 222 | 345793 | 16 |
| CSYAGSSTWV | 12 | 310112 | 222 | 228385 | 24 |
| CSYAGSYTLV | 12 | 303202 | 221 | 105549 | 105 |
| CSYAGSSTYV | 12 | 289523 | 222 | 70022 | 50 |
| CSYAGSYTYV | 12 | 272377 | 217 | 66936 | 238 |
| SSYTSSSTV | 12 | 258531 | 222 | 48280 | 60 |
| SSYAGSNNVV | 12 | 234719 | 220 | 68664 | 151 |
| CSYAGSYTWV | 12 | 233216 | 222 | 167248 | 30 |
| AAWDDSLNGVV | 12 | 229228 | 221 | 166275 | 100 |
| QSADSSGTYWV | 12 | 223892 | 222 | 355314 | 14 |
| CSYAGSSTVV | 12 | 218058 | 219 | 89456 | 181 |
| QSADSSGTWV | 12 | 216189 | 222 | 169113 | 28 |
| QVWDSSSDHYV | 12 | 197702 | 220 | 92091 | 143 |
| GTWDSSLSAV | 12 | 197402 | 222 | 144293 | 32 |
| SSYTSSSTLYV | 12 | 186672 | 220 | 72660 | 149 |
| QSYDSSLSVV | 12 | 186100 | 221 | 141414 | 102 |
| CSYAGSSTFVV | 12 | 185876 | 222 | 100283 | 44 |
| MIWHSSAWV | 12 | 176053 | 222 | 213760 | 25 |
| SSYAGSNNYV | 12 | 174082 | 215 | 51916 | 278 |
| NSRDSSNHYV | 12 | 171415 | 213 | 36728 | 332 |
| AAWDDSLSGWV | 12 | 158114 | 220 | 119456 | 142 |
| SSYTSSSVV | 12 | 157208 | 216 | 56559 | 258 |
| AAWDDSLNGYV | 12 | 154209 | 220 | 78472 | 145 |
| CSYAGSSTYVV | 12 | 146130 | 213 | 43536 | 329 |
| NSRDSSGNHRV | 12 | 145505 | 222 | 133334 | 34 |
| SSYTSSSTPVV | 12 | 145361 | 221 | 166806 | 99 |
| SSYTSSSTYVV | 12 | 137193 | 217 | 48266 | 240 |
| SSYTSSSTHVV | 12 | 136872 | 221 | 73256 | 112 |
| GTWDSSLSWV | 12 | 128153 | 222 | 260170 | 20 |
| QSYDSSLSGSVV | 12 | 124130 | 222 | 124175 | 36 |
| VLYMGSGISV | 12 | 123003 | 216 | 101152 | 256 |
| SSYTSSSTLGV | 12 | 115203 | 222 | 116304 | 42 |
| SSYAGSNNFVV | 12 | 114402 | 219 | 63294 | 190 |
| QSYDSSLSGYVV | 12 | 113810 | 221 | 98251 | 107 |
| SAWDSSLSAWV | 12 | 112755 | 198 | 240087 | 606 |
| CSYAGSYTVV | 12 | 106798 | 209 | 35558 | 405 |
| QVWDSSSDHRV | 12 | 105093 | 222 | 153345 | 31 |
| AAWDDSLSGPV | 12 | 104328 | 222 | 116392 | 41 |
| SSYTSSSTLDVV | 12 | 103308 | 217 | 46750 | 241 |
| QSADSSGTYRV | 12 | 102653 | 222 | 134024 | 33 |
| SSYTSSSTL | 12 | 102244 | 220 | 55442 | 154 |
| AAWDDSLNGRV | 12 | 101243 | 222 | 118407 | 40 |
| LLSYSGARV | 12 | 100450 | 204 | 20782 | 489 |
| VLYMGSGIWV | 12 | 100325 | 217 | 160495 | 237 |
| SSYTSSSTLEV | 12 | 98847 | 222 | 76247 | 49 |
| AAWDDSLSGRV | 12 | 98649 | 222 | 95437 | 46 |
| QSADSSGTYV | 12 | 96375 | 221 | 90631 | 109 |
| QAWDSSTAV | 12 | 93094 | 221 | 89724 | 110 |
| QSADSSGTVV | 12 | 92313 | 219 | 73742 | 186 |
| CSYAGSSTFV | 12 | 91979 | 220 | 47970 | 159 |
| CSYAGSYTFVV | 12 | 86793 | 210 | 38132 | 384 |
| AAWDDSLSGVV | 12 | 83414 | 217 | 51968 | 239 |
| GTWDSSLSVV | 12 | 82160 | 222 | 84274 | 47 |
| QSYDSSNVV | 12 | 80990 | 216 | 48988 | 259 |
| CSYAGSYTFV | 12 | 80764 | 213 | 26386 | 339 |
| QTWGTGIQV | 12 | 80345 | 219 | 68461 | 187 |
| SSYAGSNNFV | 12 | 76267 | 215 | 24579 | 284 |
| QSYDSSLSDVV | 12 | 75572 | 213 | 43040 | 331 |
| SSYAGSNNWV | 12 | 75523 | 214 | 57075 | 301 |
| GTWDSSLSAEV | 12 | 74284 | 222 | 168903 | 29 |
| QSYDSSLSGSRV | 12 | 73352 | 222 | 183383 | 26 |
| MIWHSSAVV | 12 | 73152 | 220 | 78518 | 144 |
| CSYAGSYV | 12 | 71679 | 189 | 15732 | 761 |
| GTWDSSLSVWV | 12 | 68501 | 221 | 154114 | 101 |
| ETWDSNTRV | 12 | 67024 | 214 | 93555 | 299 |
| GTWDSSLSAGGV | 12 | 66383 | 222 | 171020 | 27 |
| GTWDSSLSDVV | 12 | 66328 | 217 | 34774 | 244 |
| QSYDSSNQV | 12 | 65311 | 213 | 43168 | 330 |
| CSYAGSYTV | 12 | 63716 | 180 | 12446 | 924 |
| SSYTSSSTPYV | 12 | 63648 | 220 | 52526 | 157 |
| SSYTSSSTPV | 12 | 62711 | 222 | 66743 | 51 |
| GTWDSSLSAGRV | 12 | 62397 | 222 | 109508 | 43 |
| NSRDSSGNHLVV | 12 | 60317 | 219 | 46450 | 194 |

TRA:

| | | | Adults | Adult | |
|---|---|---|---|---|---|
| | CB with | CB total | with | total | Adult |
| CDR3 | CDR3 | reads | CDR3 | reads | rank |
| * | 12 | 5706530 | 222 | 31630222 | 0 |
| VVSDRGSTLGRLY | 12 | 101761 | 218 | 420007 | 238 |
| AVNTGGFKTI | 12 | 67618 | 222 | 198140 | 8 |
| AVNDYKLS | 12 | 57875 | 222 | 125653 | 11 |
| AVNQAGTALI | 12 | 53821 | 222 | 173459 | 9 |
| AVNTGFQKLV | 12 | 52553 | 222 | 126814 | 10 |
| AVNSGGYQKVT | 12 | 52104 | 222 | 121406 | 12 |
| AVNTNAGKST | 12 | 41151 | 222 | 99401 | 15 |
| AATDSWGKLQ | 12 | 33128 | 208 | 20479 | 775 |
| AVRDTGGFKTI | 12 | 31718 | 220 | 46581 | 145 |
| AVMDSSYKLI | 12 | 29126 | 221 | 618800 | 70 |
| AVDTGRRALT | 12 | 28633 | 222 | 79774 | 23 |
| AVNRDDKII | 12 | 28598 | 222 | 98854 | 16 |
| AVNSGYSTLT | 12 | 27663 | 222 | 69962 | 28 |
| AVRDTGRRALT | 12 | 26874 | 221 | 31472 | 122 |
| AVNTGNQFY | 12 | 26839 | 222 | 98561 | 17 |
| AVTGNQFY | 12 | 26234 | 222 | 57859 | 37 |
| VVNTNAGKST | 12 | 24281 | 221 | 54397 | 88 |
| AVDNYGQNFV | 12 | 23930 | 220 | 104758 | 142 |
| AVKAAGNKLT | 12 | 23078 | 222 | 54908 | 40 |
| AVYNFNKFY | 12 | 23061 | 221 | 46850 | 96 |
| AVDSNYQLI | 12 | 23035 | 222 | 99802 | 14 |
| AVSNDYKLS | 12 | 22888 | 221 | 92075 | 76 |
| VVNQAGTALI | 12 | 22323 | 221 | 42907 | 104 |
| AASNDYKLS | 12 | 21842 | 222 | 71143 | 27 |
| AVRDNYGQNFV | 12 | 21076 | 221 | 76903 | 80 |
| AVSGSARQLT | 12 | 20928 | 222 | 84917 | 21 |
| AVNYGGSQGNLI | 12 | 20567 | 222 | 76167 | 25 |
| AVNNAGNMLT | 12 | 20421 | 222 | 76805 | 24 |
| AASGGSYIPT | 12 | 19916 | 222 | 55206 | 39 |
| AVNSGNTPLV | 12 | 19818 | 221 | 53299 | 89 |
| AVRNTGGFKTI | 12 | 19796 | 222 | 50632 | 47 |
| AVSGGSYIPT | 12 | 19425 | 222 | 65402 | 31 |
| AVFSGGYNKLI | 12 | 19180 | 206 | 22775 | 884 |
| AVSGGYQKVT | 12 | 18706 | 222 | 52480 | 45 |
| AASTGGFKTI | 12 | 18376 | 219 | 53810 | 195 |
| AVRDQAGTALI | 12 | 18360 | 212 | 22551 | 563 |
| AASKGGSYIPT | 12 | 17748 | 221 | 84938 | 77 |
| ALNTGGFKTI | 12 | 17696 | 222 | 64776 | 33 |
| AVMDSNYQLI | 12 | 16827 | 222 | 2419871 | 2 |
| AVNSGGSNYKLT | 12 | 16633 | 222 | 87485 | 20 |
| AVNTDKLI | 12 | 16320 | 222 | 61383 | 36 |
| AVRDDKII | 12 | 15520 | 222 | 98215 | 18 |
| ALYNFNKFY | 12 | 15469 | 221 | 57631 | 84 |
| AVSGNTPLV | 12 | 15430 | 219 | 45088 | 197 |
| AASTSGTYKYI | 12 | 15306 | 222 | 61388 | 35 |
| AVYTGGFKTI | 12 | 15243 | 222 | 71325 | 26 |
| AVASGGSYIPT | 12 | 15231 | 221 | 54756 | 87 |
| AVDTGGFKTI | 12 | 15177 | 222 | 51993 | 46 |
| AVTSGTYKYI | 12 | 15156 | 222 | 114374 | 13 |
| AVSDTGGFKTI | 12 | 15053 | 222 | 34204 | 62 |
| AVYSSASKII | 12 | 14972 | 221 | 55926 | 85 |
| AVIKAAGNKLT | 12 | 14919 | 218 | 41000 | 245 |
| VVNDYKLS | 12 | 14851 | 213 | 32473 | 491 |
| AVLNQAGTALI | 12 | 14636 | 222 | 56941 | 38 |
| AANDYKLS | 12 | 14583 | 219 | 51774 | 196 |
| AVRGGSYIPT | 12 | 14298 | 220 | 35627 | 161 |
| LVGDTGRRALT | 12 | 14256 | 201 | 13886 | 1203 |
| AVRDDYKLS | 12 | 14170 | 219 | 36066 | 202 |
| AVDDYKLS | 12 | 14168 | 218 | 48006 | 243 |
| ALNDYKLS | 12 | 14163 | 222 | 52490 | 44 |
| AVRSNDYKLS | 12 | 14087 | 222 | 45064 | 48 |
| AVRSGGSYIPT | 12 | 14009 | 218 | 30467 | 257 |
| AVRDSNYQLI | 12 | 13985 | 222 | 2484161 | 1 |
| AVGGSQGNLI | 12 | 13817 | 222 | 87944 | 19 |
| AVTGGGNKLT | 12 | 13738 | 221 | 46436 | 99 |
| VVNTGGFKTI | 12 | 13680 | 218 | 27505 | 268 |
| AVPNQAGTALI | 12 | 13633 | 221 | 83407 | 78 |
| VVNTGFQKLV | 12 | 13598 | 215 | 33475 | 388 |
| AANTGGFKTI | 12 | 13545 | 220 | 53160 | 143 |
| AVNFGNEKLT | 12 | 13310 | 221 | 78840 | 79 |
| AVRMDSSYKLI | 12 | 13300 | 220 | 35663 | 160 |
| AVSSNDYKLS | 12 | 12986 | 222 | 44704 | 49 |
| AVSNQAGTALI | 12 | 12762 | 221 | 52716 | 90 |
| AVTGGFKTI | 12 | 12711 | 218 | 27004 | 269 |
| AVEDTGGFKTI | 12 | 12710 | 210 | 23583 | 657 |
| AVGSSNTGKLI | 12 | 12669 | 209 | 56824 | 713 |
| AASNQAGTALI | 12 | 12585 | 221 | 50554 | 93 |
| AANFGNEKLT | 12 | 12435 | 222 | 62582 | 34 |
| AVHGSSNTGKLI | 12 | 12423 | 209 | 51848 | 714 |
| AANQAGTALI | 12 | 12401 | 222 | 67275 | 29 |
| AVNAGNNRKLI | 12 | 12187 | 222 | 54337 | 41 |
| AVSGGYNKLI | 12 | 12128 | 198 | 14223 | 1411 |
| AGGSQGNLI | 12 | 12125 | 220 | 48617 | 144 |
| AVGSNDYKLS | 12 | 12050 | 221 | 36521 | 112 |
| AVYNNNDMR | 12 | 11972 | 218 | 31554 | 254 |
| AVANQAGTALI | 12 | 11821 | 220 | 36119 | 159 |
| AVDRGSTLGRLY | 12 | 11694 | 222 | 33576 | 63 |
| AVTTDSWGKLQ | 12 | 11642 | 205 | 78122 | 933 |
| ALDTGRRALT | 12 | 11586 | 219 | 35213 | 204 |
| AVNSNSGYALN | 12 | 11269 | 213 | 40209 | 490 |
| AVSSGGYQKVT | 12 | 11067 | 221 | 34524 | 114 |
| AATSGTYKYI | 12 | 11013 | 217 | 31529 | 304 |
| AVNNQAGTALI | 12 | 10970 | 218 | 35422 | 250 |
| AVEDTGRRALT | 12 | 10963 | 205 | 19566 | 939 |
| AVRGSQGNLI | 12 | 10808 | 221 | 51440 | 92 |
| AVNNYGQNFV | 12 | 10750 | 220 | 42657 | 150 |
| AVNNFNKFY | 12 | 10646 | 218 | 31132 | 255 |
| AVRDSGGYQKVT | 12 | 10537 | 214 | 22536 | 435 |

TRB:

| | | | Adults | Adult | |
|---|---|---|---|---|---|
| | CB with | CB total | with | total | Adult |
| CDR3 | CDR3 | reads | CDR3 | reads | rank |
| * | 30 | 10045379 | 222 | 3232641 | 0 |
| ASSLGQNTEAF | 30 | 93056 | 206 | 10690 | 5 |
| ASSLAGGTDTQY | 30 | 88853 | 173 | 5647 | 64 |
| ASSLGYEQY | 30 | 82685 | 199 | 10688 | 13 |
| ASSLQNTEAF | 30 | 81161 | 141 | 4719 | 228 |
| ASSLADTQY | 30 | 69188 | 181 | 7614 | 45 |
| ASSLGNTEAF | 30 | 62782 | 198 | 10130 | 14 |
| ASSLGGTEAF | 30 | 58963 | 203 | 10995 | 8 |
| ASSLGTGGYEQY | 30 | 54512 | 118 | 2311 | 549 |
| ASSLQGNQPQH | 30 | 52206 | 173 | 6309 | 63 |
| ASSFTDTQY | 30 | 51198 | 192 | 7701 | 25 |
| ASSLTDTQY | 30 | 50876 | 207 | 12809 | 4 |
| ASSQETQY | 30 | 50375 | 184 | 7316 | 39 |
| ASSLSYEQY | 30 | 46016 | 205 | 12321 | 6 |
| ASSLEETQY | 30 | 45458 | 184 | 8580 | 37 |
| ASSLGGYEQY | 30 | 44934 | 192 | 7268 | 26 |
| ASSLAGGPDTQY | 30 | 43546 | 78 | 1353 | 1927 |
| ASSLTGNTEAF | 30 | 43428 | 196 | 9003 | 20 |
| ASSLGGTDTQY | 30 | 41732 | 184 | 6508 | 41 |
| ASSLNTEAF | 30 | 41459 | 212 | 13823 | 1 |
| ASSSSYEQY | 30 | 39636 | 193 | 12057 | 21 |
| ASSLDTYEQY | 30 | 39387 | 78 | 1312 | 1931 |
| ASSPSTDTQY | 30 | 38520 | 208 | 11606 | 3 |
| ASSLGQGYEQY | 30 | 37663 | 166 | 7473 | 87 |
| ASSLTGGYEQY | 30 | 37280 | 92 | 1394 | 1241 |
| ASSLGTDTQY | 30 | 36873 | 192 | 8503 | 24 |
| ASSLAGTDTQY | 30 | 35543 | 149 | 4325 | 167 |
| ASSLDSSYEQY | 30 | 35499 | 136 | 3246 | 276 |
| ASSLDSNQPQH | 30 | 35364 | 201 | 9697 | 10 |
| ASSLSTDTQY | 30 | 34516 | 197 | 12688 | 18 |
| SARQGNQPQH | 30 | 34339 | 96 | 2252 | 1051 |
| ASSLDSYEQY | 30 | 34277 | 166 | 5026 | 90 |
| ASSSTDTQY | 30 | 33841 | 197 | 12875 | 17 |
| ASSLGGNQPQH | 30 | 32531 | 201 | 9444 | 11 |
| ASSLDSTDTQY | 30 | 32313 | 122 | 3154 | 463 |
| ASSLTSGTDTQY | 30 | 32211 | 94 | 1798 | 1146 |
| ASSLGTEAF | 30 | 31798 | 191 | 9278 | 27 |
| ASSYSYEQY | 30 | 31559 | 184 | 7369 | 38 |
| ASSQGYEQY | 30 | 31387 | 173 | 6693 | 62 |
| ASSLQGNTEAF | 30 | 30158 | 205 | 9290 | 7 |
| ASSLTGGTEAF | 30 | 29963 | 178 | 5589 | 50 |
| ASSLGYGYT | 30 | 29830 | 160 | 5835 | 113 |
| ASSLGGNTEAF | 30 | 29808 | 209 | 78580 | 2 |
| ASSLQGYEQY | 30 | 29048 | 133 | 3091 | 309 |
| ASSLGQGTDTQY | 30 | 29009 | 140 | 3385 | 239 |
| ASSLGETQY | 30 | 28449 | 187 | 8315 | 34 |
| ASSLTGGTDTQY | 30 | 28091 | 104 | 1984 | 849 |
| ASSLLAGGTDTQY | 30 | 28007 | 124 | 3805 | 416 |
| ASSLLAGTDTQY | 30 | 27588 | 69 | 1389 | 2597 |
| ASSLAYEQY | 30 | 27429 | 158 | 5202 | 123 |
| ASSSQETQY | 30 | 26896 | 190 | 7476 | 31 |
| ASSPSYEQY | 30 | 26730 | 196 | 22288 | 19 |
| ASSLGGEQY | 30 | 26639 | 144 | 5224 | 203 |
| ASSLTSTDTQY | 30 | 26468 | 99 | 2016 | 976 |
| ASSLGDTQY | 30 | 25457 | 178 | 6761 | 48 |
| ASSLSSYEQY | 30 | 24961 | 190 | 8112 | 30 |
| ASSYTDTQY | 30 | 24354 | 166 | 5723 | 88 |
| ASSLASTDTQY | 30 | 24231 | 124 | 2879 | 421 |
| ASSLGQNYGYT | 30 | 24043 | 177 | 7841 | 52 |
| ASSYSTDTQY | 30 | 23958 | 122 | 3020 | 464 |
| ASSLAGGSYEQY | 30 | 23501 | 149 | 3581 | 170 |
| ASSRTDTQY | 30 | 23466 | 167 | 9327 | 80 |
| ASSYTYEQY | 30 | 23005 | 71 | 961 | 2482 |
| ASSLAGYEQY | 30 | 22571 | 158 | 3810 | 125 |
| ASSFYNEQF | 30 | 22314 | 139 | 5282 | 243 |
| ASSLTGYEQY | 30 | 21854 | 117 | 2682 | 562 |
| ASSRDTYEQY | 30 | 21685 | 72 | 1335 | 2354 |
| ASRQGNQPQH | 30 | 21336 | 87 | 1998 | 1428 |
| ASSLAGGQETQY | 30 | 21319 | 115 | 2739 | 590 |
| ASSLGSYEQY | 30 | 21315 | 191 | 6203 | 28 |
| ASSSYEQY | 30 | 20503 | 125 | 3569 | 401 |
| ASSLAGNTEAF | 30 | 20132 | 179 | 5619 | 47 |
| ASSPQETQY | 30 | 19624 | 186 | 8112 | 35 |
| SARLAGGTDTQY | 30 | 19578 | 56 | 1085 | 4265 |
| ASSLTTDTQY | 30 | 19151 | 137 | 3180 | 265 |
| ASSLDRNTEAF | 30 | 18871 | 193 | 7350 | 23 |
| ASSPGQNTEAF | 30 | 18604 | 170 | 5238 | 72 |
| ASSYSNQPQH | 30 | 18470 | 148 | 5663 | 175 |
| ASSQDRGYEQY | 30 | 18068 | 112 | 1997 | 655 |
| ASSLGGSNQPQH | 30 | 17971 | 176 | 5953 | 55 |
| ASSLTGNQPQH | 30 | 17702 | 167 | 5567 | 82 |
| ASSLAGGTEAF | 30 | 17572 | 163 | 5934 | 98 |
| ASSLTGYGYT | 30 | 17419 | 106 | 2265 | 785 |
| ASSYSSYEQY | 30 | 16930 | 116 | 2641 | 572 |
| ASSGQGNQPQH | 30 | 16905 | 110 | 2475 | 686 |
| ASSLANYGYT | 30 | 16718 | 109 | 2223 | 716 |
| ASSLAGAYEQY | 30 | 16662 | 120 | 2930 | 492 |
| ASSLKETQY | 30 | 16445 | 163 | 6186 | 97 |
| ASSPGQGAYEQY | 30 | 16393 | 151 | 4101 | 161 |
| ASSLGGSTDTQY | 30 | 16234 | 173 | 4748 | 67 |
| ASSLDRDTEAF | 30 | 16174 | 131 | 2258 | 336 |
| ASSFSNQPQH | 30 | 16061 | 161 | 5761 | 109 |
| ASSRTGGYEQY | 30 | 15546 | 91 | 1588 | 1278 |
| ASRDSNQPQH | 30 | 15312 | 128 | 5396 | 359 |
| ASSLLAGGYNEQF | 30 | 14845 | 110 | 2395 | 687 |
| ASSLQGAYEQY | 30 | 14841 | 98 | 1825 | 1012 |
| ASSLGVNTEAF | 30 | 14681 | 183 | 5966 | 42 |
| ASSLGLNTEAF | 30 | 14660 | 198 | 7059 | 16 |
| ASSLLAGGPDTQY | 30 | 14571 | 53 | 921 | 4864 |

TRD:

| | | | Adults | Adult | |
|---|---|---|---|---|---|
| | CB with | CB total | with | total | Adult |
| CDR3 | CDR3 | reads | CDR3 | reads | rank |
| * | 30 | 227046 | 222 | 542467 | 0 |
| ACDWGSSWDTRQMF | 26 | 62862 | 2 | 31 | 16447 |
| ACDTGGYTDKLI | 25 | 30924 | 16 | 296 | 396 |
| ACDTGGYADKLI | 24 | 4681 | 2 | 28 | 16836 |
| ACDTGGYSWDTRQMF | 23 | 11459 | 4 | 165 | 3705 |
| ACDILGDTDKLI | 22 | 11178 | 83 | 6717 | 5 |
| ACDVLGDTDKLI | 22 | 9191 | 76 | 5983 | 10 |
| ACDWGSSWDTRQMS | 22 | 196 | 1 | 1 | 556003 |
| ACDWGSS*DTRQMF | 22 | 111 | | | |
| ACDILGDTAQLF | 21 | 4661 | 14 | 296 | 493 |
| ACDILGDTLTAQLF | 21 | 4225 | 6 | 297 | 1913 |
| ACDTAGGYSWDTRQMF | 21 | 3921 | 23 | 1420 | 161 |
| ACDVLGDTAQLF | 21 | 3778 | 9 | 846 | 977 |
| ACDTGGYLTAQLF | 21 | 3053 | 2 | 47 | 14836 |
| ACGWGSSWDTRQMF | 21 | 228 | | | |
| ACDILGDTWDTRQMF | 20 | 3600 | 4 | 83 | 3957 |
| ACDTVLGDTSSWDTRQMF | 20 | 1380 | 51 | 2342 | 22 |
| ACDWGGSWDTRQMF | 20 | 375 | | | |
| ACDRGSSWDTRQMF | 20 | 287 | | | |
| ACDWGSSWDTRQML | 20 | 229 | | | |
| ACDWGSPWDTRQMF | 20 | 213 | | | |
| ACDTGGYWDTRQMF | 19 | 7611 | | | |
| ACDTWGMTAQLF | 19 | 5958 | 4 | 96 | 3895 |
| ACDTVLGDTWDTRQMF | 19 | 5546 | 30 | 1163 | 89 |
| ACDTWDTRQMF | 19 | 3762 | 15 | 673 | 426 |
| ACDTWGSSWDTRQMF | 19 | 2952 | 7 | 106 | 1645 |
| ACDTWGYTDKLI | 19 | 2808 | 29 | 5325 | 95 |
| ACDTVLGDSSWDTRQMF | 19 | 2355 | 50 | 2193 | 24 |
| ACDVLGDTWDTRQMF | 19 | 1873 | 4 | 40 | 4368 |
| ACDWGSSWGTRQMF | 19 | 246 | | | |
| ACDWGSSWDTRQVF | 19 | 225 | | | |
| ACDWGSSWDARQMF | 19 | 204 | | | |
| ACDWGSSWDTRRMF | 19 | 127 | | | |
| ACDTWGTAQLF | 18 | 4135 | 10 | 174 | 888 |
| ACDTVGDTDKLI | 18 | 3924 | 95 | 9152 | 3 |
| ACDTGGYSSWDTRQMF | 18 | 2948 | 29 | 1743 | 96 |
| ACDTLGDTLTAQLF | 18 | 2561 | 11 | 1464 | 693 |
| ACDTGGYGSWDTRQMF | 18 | 974 | 42 | 1005 | 41 |
| ACDWGSSRDTRQMF | 18 | 202 | | | |
| ARDWGSSWDTRQMF | 18 | 183 | | | |
| ACVWGSSWDTRQMF | 18 | 169 | | | |
| ACDTGGLTAQLF | 17 | 4806 | | | |
| ACDTGGSWDTRQMF | 17 | 3287 | 1 | 1 | 702983 |
| ACDWGTWDTRQMF | 17 | 3130 | | | |
| ACDILGDLTAQLF | 17 | 2853 | 6 | 129 | 2028 |
| ACDTVLGDSWDTRQMF | 17 | 2453 | 19 | 762 | 253 |
| ACDWGSSWDT*QMF | 17 | 100 | | | |
| ACDTGGYTDKPI | 17 | 83 | | | |
| ACDWGSSWDTQQMF | 17 | 52 | | | |
| ACDWGCSWDTRQMF | 17 | 50 | | | |
| ACDYWGSSWDTRQMF | 16 | 2786 | 1 | 1 | 791893 |
| ACDLLGDTDKLI | 16 | 2082 | 66 | 4529 | 13 |
| ACDSTGGSWDTRQMF | 16 | 1822 | 20 | 2027 | 226 |
| ACDTLGDTDKLI | 16 | 1790 | 141 | 29665 | 1 |
| ACDVLGDSSWDTRQMF | 16 | 1657 | 13 | 418 | 542 |
| ACDWGNSWDTRQMF | 16 | 898 | | | |
| ACDWGSSWDTRQTF | 16 | 142 | | | |
| ACDWGSAWDTRQMF | 16 | 138 | | | |
| ACDWGSSCDTRQMF | 16 | 137 | 1 | 1 | 1117353 |
| ACGTGGYTDKLI | 16 | 123 | 1 | 3 | 327812 |
| ACDAGGYTDKLI | 16 | 118 | | | |
| ACDWGSSWDTRLMF | 16 | 82 | | | |
| ACDGGSSWDTRQMF | 16 | 74 | 1 | 18 | 109500 |
| GCDWGSSWDTRQMF | 16 | 64 | | | |
| ACDWESSWDTRQMF | 16 | 57 | | | |
| ACDLGSSWDTRQMF | 16 | 54 | | | |
| ACDWGSSWDTREMF | 16 | 50 | | | |
| ACDVLGDTLTAQLF | 15 | 2866 | 6 | 94 | 2097 |
| ACDTAGGSWDTRQMF | 15 | 2377 | 33 | 4457 | 68 |
| ACDVLGDLTAQLF | 15 | 2023 | 5 | 66 | 2872 |
| ACDNTGGYSWDTRQMF | 15 | 1300 | 7 | 129 | 1603 |
| ACDTVGGYSWDTRQMF | 15 | 1174 | 4 | 36 | 4421 |
| AFTGGYWDTRQMF | 15 | 1148 | 1 | 2 | 364662 |
| ACDTAGGYWDTRQMF | 15 | 1084 | 8 | 169 | 1280 |
| AFTGGYTDKLI | 15 | 1073 | 4 | 78 | 4003 |
| ACDTVGDTLTAQLF | 15 | 904 | 6 | 178 | 1980 |
| ACDTWGLTAQLF | 15 | 842 | | | |
| ACDWGIRSWDTRQMF | 15 | 566 | | | |
| ACDTLGDSSWDTRQMF | 15 | 552 | 26 | 1994 | 120 |
| ACDTGVYTDKLI | 15 | 458 | 2 | 2 | 34255 |
| ACDSGGYTDKLI | 15 | 377 | 2 | 2 | 28498 |
| ACDTGGYSDKLI | 15 | 243 | 1 | 6 | 244415 |
| TCDWGSSWDTRQMF | 15 | 111 | | | |
| ACDTGGHTDKLI | 15 | 100 | 2 | 2 | 28566 |
| ARDTGGYTDKLI | 15 | 99 | 2 | 3 | 27952 |
| ACDWGSSWDTRQLF | 15 | 82 | | | |
| ACDWGSSWDTRQIF | 15 | 79 | | | |
| ACDWGSSWDSRQMF | 15 | 72 | | | |
| ACDTGGCTDKLI | 15 | 67 | | | |
| ACDWGSCWDTRQMF | 15 | 60 | | | |
| ACDWRSSWDTRQMF | 15 | 51 | | | |
| AFDWGSSWDTRQMF | 15 | 45 | 1 | 1 | 722530 |
| ACDWGSSWDTRQMV | 15 | 43 | | | |
| ACDTGGYAAQLF | 14 | 1693 | | | |
| ACDILGDSSWDTRQMF | 14 | 1635 | 14 | 244 | 502 |
| AFTGGYSWDTRQMF | 14 | 1583 | 2 | 155 | 12100 |
| AFGGYTDKLI | 14 | 1428 | 5 | 65478 | 2442 |
| ACDTGGYASWDTRQMF | 14 | 1339 | 14 | 576 | 470 |
| ACDILGDTTAQLF | 14 | 899 | 2 | 36 | 15889 |

TRG:

| | | | Adults | Adult | |
|---|---|---|---|---|---|
| | CB with | CB total | with | total | Adult |
| CDR3 | CDR3 | reads | CDR3 | reads | rank |
| * | 12 | 527036 | 222 | 10972664 | 0 |
| ALWEVQELGKKIKV | 12 | 19295 | 220 | 930787 | 1 |
| ATWDTTGWFKI | 12 | 12462 | 193 | 21628 | 68 |
| ATWDYYKKL | 12 | 10231 | 214 | 45235 | 9 |
| ATWDGYYKKL | 12 | 9663 | 217 | 62279 | 4 |
| ATWDGNYYKKL | 12 | 5790 | 216 | 159487 | 5 |
| ATWDGPYYKKL | 12 | 5403 | 214 | 36592 | 10 |
| ATWDYKKL | 12 | 5064 | 212 | 40158 | 16 |
| ATWDGYKKL | 12 | 4690 | 197 | 39035 | 52 |
| ATWDGRYKKL | 12 | 4455 | 209 | 40700 | 22 |
| ATWDGPNYYKKL | 12 | 4077 | 205 | 24307 | 27 |
| ATWDGPYKKL | 12 | 3716 | 201 | 25216 | 37 |
| ATWDGLYYKKL | 12 | 3521 | 211 | 49650 | 19 |
| ATWDKKL | 12 | 3367 | 206 | 24521 | 25 |
| ATWDGRYYKKL | 12 | 2958 | 204 | 26665 | 28 |
| ATWDGHYKKL | 12 | 2727 | 193 | 28319 | 67 |
| ATWDRPYYKKL | 12 | 2491 | 195 | 16983 | 60 |
| ALWEVNYYKKL | 12 | 2438 | 199 | 35940 | 45 |
| ATWDSSDWIKT | 12 | 2105 | 171 | 8512 | 158 |
| ATWDGPGYYKKL | 12 | 2103 | 187 | 17680 | 90 |
| ALWEVYYKKL | 12 | 2091 | 189 | 26888 | 75 |
| ATWNYYKKL | 12 | 1753 | 171 | 10768 | 157 |
| ATWDGSDWIKT | 12 | 1713 | 160 | 9519 | 218 |
| ATWDGSSDWIKT | 12 | 1629 | 172 | 13463 | 147 |
| ATWDGGYKKL | 12 | 1621 | 162 | 46994 | 205 |
| ATWENYYKKL | 12 | 1587 | 187 | 61856 | 89 |
| ATWDDYKKL | 12 | 1582 | 185 | 10485 | 100 |
| ATWGYYKKL | 12 | 1527 | 181 | 27458 | 117 |
| ATWDGLYKKL | 12 | 1520 | 193 | 13754 | 69 |
| ATWDATGWFKI | 12 | 1496 | 92 | 4298 | 968 |
| ATWDGRNYYKKL | 12 | 1412 | 183 | 11839 | 108 |
| ATWDGRKKL | 12 | 1367 | 168 | 12786 | 172 |
| ATWDGPGWFKI | 12 | 1342 | 110 | 2909 | 663 |
| ATWDSYKKL | 12 | 1318 | 172 | 12452 | 148 |
| ATWDRLYYKKL | 12 | 1286 | 187 | 15783 | 91 |
| ATWDGLGYKKL | 12 | 1279 | 170 | 11679 | 162 |
| ATWDGFYYKKL | 12 | 1131 | 158 | 8467 | 231 |
| ATWDLYYKKL | 12 | 1056 | 167 | 5780 | 179 |
| ATWDGYSSDWIKT | 12 | 995 | 135 | 5093 | 394 |
| ATWDGGYYKKL | 12 | 989 | 166 | 9291 | 185 |
| ATWVNYYKKL | 12 | 915 | 159 | 4927 | 224 |
| ATWDGPSDWIKT | 12 | 865 | 130 | 39243 | 439 |
| ATWYYKKL | 12 | 813 | 146 | 2795 | 304 |
| ATWDSNYYKKL | 12 | 800 | 182 | 10529 | 113 |
| ATWDGTYYKKL | 12 | 795 | 155 | 5227 | 250 |
| ATWDGRDYYKKL | 12 | 723 | 122 | 2533 | 524 |
| ATWDGQNYYKKL | 12 | 692 | 168 | 25721 | 170 |
| ATWDEKL | 12 | 639 | 133 | 104546 | 410 |
| ATWDSTGWFKI | 12 | 392 | 102 | 1197 | 787 |
| ATCDYYKKL | 12 | 117 | 144 | 2727 | 320 |
| ATWDGPGYKKL | 11 | 3095 | 202 | 29370 | 34 |
| ATWDGPKKL | 11 | 2768 | 183 | 17810 | 107 |
| ATWDRNYYKKL | 11 | 2540 | 204 | 22662 | 29 |
| ATWDSYYKKL | 11 | 1996 | 199 | 14283 | 48 |
| ATWDGKKL | 11 | 1970 | 195 | 37653 | 59 |
| ATWDRYKKL | 11 | 1872 | 180 | 10769 | 120 |
| ATWDGDYYKKL | 11 | 1871 | 187 | 9735 | 93 |
| ATWDGPTGWFKI | 11 | 1634 | 113 | 4826 | 633 |
| ATWDRRYYKKL | 11 | 1614 | 189 | 9538 | 77 |
| ATWDGPRYKKL | 11 | 1607 | 176 | 10753 | 134 |
| ATWDGWFKI | 11 | 1559 | 87 | 2372 | 1098 |
| ATWDDYYKKL | 11 | 1539 | 171 | 23523 | 155 |
| ATWDGSYYKKL | 11 | 1509 | 176 | 9305 | 135 |
| ATWDRPNYYKKL | 11 | 1463 | 179 | 12300 | 128 |
| ATWDRYYKKL | 11 | 1351 | 189 | 13497 | 76 |
| ATWDGNYKKL | 11 | 1339 | 168 | 10179 | 173 |
| ATWDRPGYKKL | 11 | 1304 | 186 | 6708 | 96 |
| ATWDGSNYYKKL | 11 | 1287 | 180 | 7079 | 121 |
| ATWDGRGYKKL | 11 | 1279 | 183 | 17925 | 106 |
| ATWDGPEKL | 11 | 1165 | 154 | 10024 | 255 |
| ATWDGQGYKKL | 11 | 1145 | 131 | 4387 | 428 |
| ATWDGLNYYKKL | 11 | 1100 | 199 | 31905 | 46 |
| ATWDENYYKKL | 11 | 1086 | 165 | 12485 | 190 |
| ATWDGYYYKKL | 11 | 1072 | 188 | 10589 | 85 |
| ATWDGDKKL | 11 | 1058 | 137 | 6488 | 372 |
| ATWDGPNYKKL | 11 | 1044 | 165 | 6346 | 191 |
| ATWDGYTTGWFKI | 11 | 1043 | 118 | 2684 | 572 |
| ATWGNYYKKL | 11 | 1017 | 161 | 9120 | 216 |
| ATWDVYYKKL | 11 | 966 | 187 | 10452 | 92 |
| ATWDGTGWFKI | 11 | 937 | 91 | 3791 | 990 |
| ALWEDYYKKL | 11 | 927 | 169 | 17684 | 166 |
| ATWDRRDYKKL | 11 | 792 | 141 | 9128 | 341 |
| ATWDPYYKKL | 11 | 790 | 145 | 5164 | 314 |
| ATWDGIYYKKL | 11 | 784 | 181 | 123310 | 116 |
| ATWDGRDYKKL | 11 | 778 | 154 | 5940 | 256 |
| ATWDGQYYKKL | 11 | 764 | 156 | 7615 | 243 |
| ATWDGVYYKKL | 11 | 756 | 153 | 3657 | 264 |
| ATWGYKKL | 11 | 730 | 166 | 11484 | 184 |
| ALWEVKKL | 11 | 720 | 133 | 8450 | 411 |
| ATWDGSYKKL | 11 | 683 | 136 | 2687 | 384 |
| ATWEYYKKL | 11 | 674 | 168 | 4061 | 174 |
| ALWEVGYKKL | 11 | 654 | 139 | 37591 | 354 |
| ATWDRGYYKKL | 11 | 641 | 131 | 5797 | 427 |
| ATWDGPHYYKKL | 11 | 598 | 115 | 3461 | 604 |
| ATWDRRGKL | 11 | 585 | 112 | 3956 | 641 |
| ATWDGRGYYKKL | 11 | 551 | 153 | 15770 | 261 |
| ATWDRRNYYKKL | 11 | 536 | 134 | 4647 | 406 |
| ATWDRPRYKKL | 11 | 521 | 143 | 3349 | 328 |
| ATWDGPVYKKL | 11 | 494 | 135 | 1501 | 396 |

TABLE 2
IgH:

| | Adults | Adult | | | |
|---|---|---|---|---|---|
| | with | total | CB with | CB total | |
| CDR3 | CDR3 | reads | CDR3 | reads | CB rank |
| * | 222 | 5712880 | 30 | 10026640 | 0 |
| ARDLDY | 124 | 4569 | 29 | 42355 | 23 |
| ARDFDY | 103 | 3898 | 28 | 25373 | 56 |
| ARDDY | 72 | 2383 | 28 | 21352 | 57 |
| ARGFDY | 69 | 1410 | 29 | 15010 | 31 |
| ARDSSGWYYFDY | 67 | 1697 | 30 | 45778 | 2 |
| AREDY | 67 | 1612 | 28 | 13371 | 69 |
| ARGNWFDP | 66 | 1871 | 23 | 8492 | 696 |
| ARDPDY | 66 | 1426 | 30 | 10676 | 18 |
| ARDLGY | 64 | 1334 | 28 | 13309 | 70 |
| ARGLDY | 60 | 1756 | 30 | 18643 | 9 |
| ARIGYSSSSFDY | 59 | 1855 | 6 | 576 | 56616 |
| ARGDWFDP | 56 | 1405 | 24 | 5362 | 532 |
| ARVDY | 56 | 1206 | 28 | 12619 | 72 |
| ARDYYYYGMDV | 55 | 1046 | 23 | 4552 | 738 |
| ARGDY | 53 | 1292 | 30 | 15855 | 14 |
| ARDYYYGMDV | 52 | 1300 | 24 | 6651 | 507 |
| ARDPFDY | 51 | 1420 | 22 | 8275 | 957 |
| ARDAFDI | 51 | 1157 | 30 | 32844 | 3 |
| ARGHYGMDV | 49 | 1547 | 12 | 659 | 12760 |
| ARGDAFDI | 46 | 1027 | 30 | 16700 | 10 |
| ARGYSSSWYYFDY | 46 | 944 | 29 | 46153 | 22 |
| ARDYGMDV | 44 | 1260 | 22 | 5829 | 965 |
| ARDDAFDI | 44 | 1082 | 28 | 20888 | 58 |
| ARGVDY | 44 | 597 | 30 | 10891 | 17 |
| ARNFDY | 43 | 1144 | 26 | 5055 | 269 |
| ARDGDY | 42 | 989 | 23 | 9133 | 695 |
| ARGIDY | 42 | 873 | 26 | 7492 | 236 |
| ARGYYGMDV | 42 | 814 | 18 | 3700 | 2851 |
| ARGYSSGWYYFDY | 42 | 778 | 29 | 13979 | 32 |
| AREFDY | 42 | 774 | 24 | 7718 | 501 |
| ARGDYYYGMDV | 42 | 604 | 25 | 2839 | 453 |
| ARGRYYFDY | 41 | 656 | 27 | 9468 | 128 |
| ARLDY | 40 | 968 | 26 | 5641 | 258 |
| AKDLDY | 40 | 754 | 26 | 9153 | 226 |
| ARGGYYFDY | 40 | 689 | 29 | 10059 | 38 |
| AREGY | 39 | 1036 | 28 | 8903 | 86 |
| ARGYYYYGMDV | 39 | 883 | 26 | 4359 | 279 |
| ARGWFDP | 39 | 864 | 25 | 6521 | 364 |
| ARDYYDSSGYYYFDY | 39 | 705 | 28 | 12647 | 71 |
| ARGYYYGMDV | 38 | 1115 | 19 | 8478 | 2199 |
| ARDSDY | 38 | 924 | 25 | 4994 | 389 |
| ARGYGMDV | 38 | 851 | 19 | 1663 | 2483 |
| ARDRGYFDY | 38 | 820 | 27 | 5660 | 169 |
| ARGSDY | 38 | 483 | 26 | 7157 | 241 |
| ARELDY | 37 | 806 | 28 | 7647 | 89 |
| ARGYYFDY | 37 | 803 | 27 | 10998 | 123 |
| ARGLYYFDY | 37 | 623 | 26 | 4863 | 274 |
| ARGHYGLDV | 36 | 3024 | 1 | 1 | 33137471 |
| ARDFGY | 36 | 1236 | 21 | 1751 | 1562 |
| ARDNWFDP | 36 | 766 | 22 | 3211 | 1058 |
| ARDSSSWYYFDY | 36 | 526 | 30 | 57228 | 1 |
| ARDYGGNSGWFDP | 35 | 1202 | 7 | 214 | 47174 |
| ARDSYGMDV | 35 | 1037 | 19 | 1662 | 2484 |
| ARDGY | 34 | 987 | 27 | 8840 | 133 |
| ARDYGDYYFDY | 34 | 873 | 27 | 12127 | 121 |
| ARDIDY | 34 | 855 | 21 | 5601 | 1314 |
| ARDRGWFDP | 34 | 654 | 16 | 798 | 5434 |
| ARVFDY | 34 | 607 | 24 | 5463 | 530 |
| ARDLGDY | 34 | 481 | 22 | 11468 | 953 |
| ARGSSGWYYFDY | 34 | 367 | 29 | 7516 | 47 |
| ARGRWFDP | 33 | 740 | 21 | 4432 | 1334 |
| ARGEYYFDY | 33 | 658 | 25 | 6612 | 362 |
| ARDYYGMDV | 33 | 621 | 22 | 2692 | 1091 |
| ARDSGSYYFDY | 33 | 578 | 29 | 35688 | 24 |
| ARDYGDYFDY | 32 | 684 | 26 | 11763 | 215 |
| ARAFDY | 32 | 675 | 26 | 7346 | 239 |
| ARGRNWFDP | 32 | 447 | 20 | 2198 | 1875 |
| ARDYYGDYYFDY | 31 | 1313 | 13 | 746 | 10260 |
| ARDRWFDP | 31 | 593 | 21 | 4881 | 1319 |
| ARGDYYYYGMDV | 31 | 587 | 23 | 5430 | 717 |
| ARDVDY | 31 | 522 | 27 | 8311 | 136 |
| ARDGFDY | 30 | 1322 | 22 | 5083 | 977 |
| ARSFDY | 30 | 754 | 25 | 13721 | 333 |
| AKDDY | 30 | 640 | 27 | 4412 | 186 |
| ARDYYDSSGYFDY | 30 | 529 | 26 | 7488 | 237 |
| ASLDY | 30 | 474 | 26 | 4229 | 285 |
| ARGYYDSSGYYYFDY | 30 | 450 | 29 | 7279 | 48 |
| ARIGYSSSSLDY | 29 | 1076 | 1 | 12 | 5051038 |
| ARDYDY | 29 | 698 | 28 | 9013 | 84 |
| ARGAFDI | 29 | 603 | 27 | 12216 | 120 |
| ARVGY | 29 | 548 | 23 | 4096 | 753 |
| ARDYYFDY | 29 | 491 | 22 | 4040 | 1011 |
| ARDRDAFDI | 29 | 481 | 28 | 9986 | 81 |
| TTVDY | 28 | 780 | 28 | 11285 | 76 |
| ARDPGDY | 28 | 613 | 25 | 4575 | 395 |
| ARGGDY | 28 | 571 | 25 | 7198 | 352 |
| ARDRGY | 28 | 555 | 26 | 4258 | 282 |
| ARGYCSGGSCYFDY | 28 | 479 | 28 | 14989 | 65 |
| ARDRDY | 28 | 473 | 27 | 4100 | 192 |
| ARGGY | 28 | 456 | 27 | 8380 | 135 |
| ARAYSSGWYYFDY | 28 | 448 | 28 | 5171 | 100 |
| ARDPGY | 28 | 433 | 23 | 4824 | 728 |
| ARGGWFDP | 27 | 1043 | 27 | 3664 | 197 |
| ARGPPFDY | 27 | 876 | 15 | 1873 | 5963 |
| ARGGAFDI | 27 | 680 | 27 | 4606 | 182 |
| ARGPFDY | 27 | 618 | 21 | 6165 | 1306 |
| ARGYDY | 27 | 615 | 23 | 2962 | 806 |
| ARDSSGVVYFDY | 27 | 566 | 26 | 18269 | 211 |

IgK:

| | Adults | Adult | | | |
|---|---|---|---|---|---|
| | with | total | CB with | CB total | |
| CDR3 | CDR3 | reads | CDR3 | reads | CB rank |
| * | 222 | 14114525 | 12 | 6170981 | 0 |
| QQYDNLPLT | 222 | 3317473 | 12 | 2066846 | 2 |
| QQYGSSPRT | 222 | 3303511 | 12 | 1762387 | 4 |
| QQSYSTPRT | 222 | 2952854 | 12 | 1807334 | 3 |
| QQYYSTPYT | 222 | 2681529 | 12 | 495875 | 31 |
| QQYGSSPWT | 222 | 2609412 | 12 | 1441308 | 7 |
| QQSYSTPYT | 222 | 2163695 | 12 | 3597815 | 1 |
| QQYNNWPPWT | 222 | 2005449 | 12 | 760699 | 15 |
| QQYGSSPLT | 222 | 1867304 | 12 | 1028477 | 10 |
| QQYGSSPYT | 222 | 1815657 | 12 | 1694095 | 5 |
| QQSYSTPWT | 222 | 1695770 | 12 | 1571128 | 6 |
| QQYNNWPRT | 222 | 1672121 | 12 | 663030 | 19 |
| QQRSNWPLT | 222 | 1578220 | 12 | 547580 | 27 |
| QQYNSYPWT | 222 | 1438088 | 12 | 315545 | 52 |
| QQSYSTPLT | 222 | 1303006 | 12 | 966727 | 11 |
| QQYDNLPYT | 222 | 1296252 | 12 | 1267862 | 8 |
| QQYNNWPPYT | 222 | 1272554 | 12 | 680244 | 17 |
| QQYNNWPPLT | 222 | 1203855 | 12 | 548699 | 25 |
| QQYNSYPYT | 222 | 1129544 | 12 | 485682 | 33 |
| QQYNSYPLT | 222 | 1068076 | 12 | 447639 | 37 |
| QQYYSYPRT | 222 | 1067391 | 12 | 664430 | 18 |
| QQYNNWPLT | 222 | 1031985 | 12 | 480104 | 34 |
| QQYGSSPPYT | 222 | 972444 | 12 | 691757 | 16 |
| QQSYSTPPT | 222 | 963899 | 12 | 782033 | 14 |
| QQRSNWPPIT | 222 | 896207 | 12 | 426878 | 39 |
| QQRSNWPPT | 222 | 893836 | 12 | 312412 | 54 |
| QQYNSYSWT | 222 | 876221 | 12 | 453212 | 36 |
| LQHNSYPWT | 222 | 866476 | 12 | 944829 | 12 |
| QQYYSTPLT | 222 | 862723 | 12 | 433426 | 38 |
| QQYGSSPFT | 222 | 855515 | 12 | 612647 | 23 |
| QQYDNLPIT | 222 | 850500 | 12 | 635718 | 22 |
| QQRSNWPRT | 222 | 842959 | 12 | 221567 | 69 |
| QQYNSYSRT | 222 | 836163 | 12 | 391904 | 42 |
| QQYGSSPIT | 222 | 832529 | 12 | 556768 | 24 |
| QQRSNWPPLT | 222 | 815084 | 12 | 356112 | 47 |
| LQHNSYPRT | 222 | 782633 | 12 | 544829 | 28 |
| QQRSNWPPYT | 222 | 753751 | 12 | 370863 | 44 |
| QQYGSSPPWT | 222 | 752147 | 12 | 487256 | 32 |
| QQYNSYWT | 222 | 737411 | 12 | 499060 | 30 |
| QQYGSSPQT | 222 | 726922 | 12 | 321515 | 50 |
| QQYGSSPGT | 222 | 719365 | 12 | 228566 | 66 |
| QQLNSYPLT | 222 | 718912 | 12 | 301627 | 55 |
| QQSYSTPFT | 222 | 714272 | 12 | 1186065 | 9 |
| QQYDNLPPT | 222 | 679053 | 12 | 263221 | 58 |
| QQYNNWPPIT | 222 | 674010 | 12 | 258796 | 59 |
| QQRSNWPPWT | 222 | 659585 | 12 | 225414 | 67 |
| QQYDNLPFT | 222 | 640004 | 12 | 548132 | 26 |
| MQALQTPRT | 222 | 635277 | 12 | 171478 | 98 |
| QQYGSSLWT | 222 | 597010 | 12 | 224617 | 68 |
| QQYGSSPT | 222 | 573641 | 12 | 847464 | 13 |
| MQGTHWPYT | 222 | 568891 | 12 | 102612 | 145 |
| LQHNSYPLT | 222 | 564345 | 12 | 382836 | 43 |
| QQRSNWPIT | 222 | 546612 | 12 | 230867 | 65 |
| MQALQTPLT | 222 | 543098 | 12 | 210576 | 74 |
| QQYGSSPPT | 222 | 540517 | 12 | 330406 | 49 |
| QQYDNLPRT | 222 | 536453 | 12 | 220911 | 70 |
| QQYYSTPRT | 222 | 535684 | 12 | 202753 | 79 |
| QQYYSYPLT | 222 | 525580 | 12 | 361244 | 46 |
| MQALQTPYT | 222 | 525224 | 12 | 278214 | 57 |
| QQYGSSPPIT | 222 | 514147 | 12 | 316377 | 51 |
| QQYNNWPYT | 222 | 514100 | 12 | 398122 | 40 |
| MQALQTPWT | 222 | 513198 | 12 | 186378 | 87 |
| QQSYSTPIT | 222 | 499000 | 12 | 644068 | 20 |
| QQANSFPLT | 222 | 498947 | 12 | 193699 | 83 |
| QQYYSTPVVT | 222 | 482125 | 12 | 313675 | 53 |
| QQSYSTPQT | 222 | 475924 | 12 | 211802 | 73 |
| QQYNSYSYT | 222 | 471334 | 12 | 370287 | 45 |
| QQLNSYPRT | 222 | 453795 | 12 | 182332 | 89 |
| LQDYNYPRT | 222 | 447454 | 12 | 110349 | 138 |
| QQYYSYPWT | 222 | 445511 | 12 | 393570 | 41 |
| QQSYSTLWT | 222 | 431092 | 12 | 179915 | 90 |
| QQYGSSRT | 222 | 430612 | 12 | 355708 | 48 |
| QQRSNWPWT | 222 | 429330 | 12 | 163059 | 101 |
| QQYNSYPRT | 222 | 426472 | 12 | 123957 | 127 |
| QQYGSSPPLT | 222 | 419721 | 12 | 245266 | 63 |
| QQYYSTPPT | 222 | 412231 | 12 | 172158 | 96 |
| QQYNSYST | 222 | 405406 | 12 | 523209 | 29 |
| LQHNSYPYT | 222 | 394879 | 12 | 643754 | 21 |
| QQYGSSPLYT | 222 | 379237 | 12 | 152432 | 106 |
| QQYNNWPWT | 222 | 378298 | 12 | 150588 | 109 |
| LQDYNYPWT | 222 | 362599 | 12 | 137381 | 117 |
| QQRSNWPT | 222 | 347410 | 12 | 205730 | 76 |
| QQYDNLPPYT | 222 | 346088 | 12 | 203318 | 78 |
| QQSYSTPPYT | 222 | 343511 | 12 | 254243 | 60 |
| QQYGSSLYT | 222 | 340898 | 12 | 209150 | 75 |
| QQYGSSSWT | 222 | 336774 | 12 | 111473 | 137 |
| QQYGSSPLFT | 222 | 335296 | 12 | 82549 | 167 |
| QQYYSYPYT | 222 | 333528 | 12 | 465884 | 35 |
| QQYNSYPFT | 222 | 314885 | 12 | 173418 | 95 |
| QQYGSSPKT | 222 | 313692 | 12 | 105756 | 141 |
| MQALQTPPT | 222 | 306536 | 12 | 111848 | 136 |
| QQRSNWPYT | 222 | 305522 | 12 | 148716 | 112 |
| QQRSNWPPFT | 222 | 300165 | 12 | 176371 | 92 |
| QQSYTTPRT | 222 | 292603 | 12 | 109 | 5993 |
| QQYNNWPQT | 222 | 292444 | 12 | 74218 | 175 |
| QQYYSYPPT | 222 | 291224 | 12 | 161728 | 103 |
| QQYDNLPPLT | 222 | 288444 | 12 | 212553 | 72 |
| QQYNSYPIT | 222 | 281622 | 12 | 174782 | 94 |
| QQLNSYPFT | 222 | 280897 | 12 | 193197 | 84 |

IgL:

| | Adults | Adult | | | |
|---|---|---|---|---|---|
| | with | total | CB with | CB total | |
| CDR3 | CDR3 | reads | CDR3 | reads | CB rank |
| * | 222 | 8615976 | 12 | 3459321 | 0 |
| GTWDSSLSAGV | 222 | 2656155 | 12 | 2233556 | 3 |
| GTWDSSLSAVV | 222 | 2542422 | 12 | 2953276 | 1 |
| QVWDSSSDHVV | 222 | 1009126 | 12 | 1140242 | 8 |
| SSYTSSSTLV | 222 | 921747 | 12 | 2920518 | 2 |
| QSYDSSLSGSV | 222 | 865170 | 12 | 1355919 | 5 |
| QSADSSGTYVV | 222 | 761696 | 12 | 874443 | 10 |
| GTWDSSLSAWV | 222 | 692370 | 12 | 478468 | 19 |
| QSYDSSLSGWV | 222 | 566201 | 12 | 594366 | 14 |
| SSYTSSSTLVV | 222 | 477560 | 12 | 1153503 | 7 |
| SSYTSSSTVV | 222 | 460353 | 12 | 1762737 | 4 |
| NSRDSSGNHLV | 222 | 449929 | 12 | 938981 | 9 |
| AAWDDSLNGPV | 222 | 422893 | 12 | 580611 | 15 |
| SSYTSSSTWV | 222 | 358782 | 12 | 1296277 | 6 |
| QSADSSGTYWV | 222 | 355314 | 12 | 223892 | 35 |
| NSRDSSGNHWV | 222 | 348395 | 12 | 520365 | 17 |
| QAWDSSTVV | 222 | 345793 | 12 | 321889 | 26 |
| NSRDSSGNHVV | 222 | 319786 | 12 | 710309 | 13 |
| SSYTSSSTRV | 222 | 301059 | 12 | 419596 | 22 |
| SSYAGSNNLV | 222 | 300511 | 12 | 762549 | 12 |
| GTWDSSLSWV | 222 | 260170 | 12 | 128153 | 54 |
| QVWDSSSDHPV | 222 | 247647 | 12 | 421063 | 21 |
| CSYAGSSTLV | 222 | 245335 | 12 | 478979 | 18 |
| QSYDSSLSGYV | 222 | 230769 | 12 | 555044 | 16 |
| CSYAGSSTWV | 222 | 228385 | 12 | 310112 | 27 |
| MIWHSSAWV | 222 | 213760 | 12 | 176053 | 43 |
| QSYDSSLSGSRV | 222 | 183383 | 12 | 73352 | 86 |
| GTWDSSLSAGGV | 222 | 171020 | 12 | 66383 | 91 |
| QSADSSGTWV | 222 | 169113 | 12 | 216189 | 37 |
| GTWDSSLSAEV | 222 | 168903 | 12 | 74284 | 85 |
| CSYAGSYTWV | 222 | 167248 | 12 | 233216 | 33 |
| QVWDSSSDHRV | 222 | 153345 | 12 | 105093 | 62 |
| GTWDSSLSAV | 222 | 144293 | 12 | 197402 | 39 |
| QSADSSGTYRV | 222 | 134024 | 12 | 102653 | 65 |
| NSRDSSGNHRV | 222 | 133334 | 12 | 145505 | 50 |
| GTWDNSLSAGV | 222 | 125120 | 12 | 1826 | 937 |
| QSYDSSLSGSVV | 222 | 124175 | 12 | 124130 | 55 |
| GTWDSSLSAYV | 222 | 122965 | 12 | 386293 | 25 |
| QSYDSSLSGVV | 222 | 122678 | 12 | 394146 | 24 |
| QTWGTGIRV | 222 | 121836 | 12 | 53275 | 105 |
| AAWDDSLNGRV | 222 | 118407 | 12 | 101243 | 67 |
| AAWDDSLSGPV | 222 | 116392 | 12 | 104328 | 63 |
| SSYTSSSTLGV | 222 | 116304 | 12 | 115203 | 57 |
| GTWDSSLSAGRV | 222 | 109508 | 12 | 62397 | 97 |
| CSYAGSSTFVV | 222 | 100283 | 12 | 185876 | 42 |
| GTWDSSLSARV | 222 | 97939 | 12 | 48442 | 112 |
| AAWDDSLSGRV | 222 | 95437 | 12 | 98649 | 71 |
| GTWDSSLSVV | 222 | 84274 | 12 | 82160 | 78 |
| GTWDSSLSAAV | 222 | 80962 | 12 | 48410 | 113 |
| SSYTSSSTLEV | 222 | 76247 | 12 | 98847 | 70 |
| CSYAGSSTYV | 222 | 70022 | 12 | 289523 | 29 |
| SSYTSSSTPV | 222 | 66743 | 12 | 62711 | 96 |
| GTWDGSLSAGV | 222 | 65246 | 12 | 3998 | 631 |
| QVWDSSSDLVV | 222 | 62924 | 12 | 24039 | 179 |
| GTWDSSLSALV | 222 | 61750 | 12 | 17853 | 229 |
| GTWDSSLSGGV | 222 | 56973 | 12 | 8842 | 391 |
| QSYDSSLSGRV | 222 | 55285 | 12 | 22055 | 191 |
| QSYDSSLSGLV | 222 | 55179 | 12 | 37883 | 134 |
| GTWDSSLRAGV | 222 | 51662 | 12 | 4629 | 589 |
| GAWDSSLSAVV | 222 | 51434 | 12 | 10459 | 334 |
| SSYTSSSTV | 222 | 48280 | 12 | 258531 | 31 |
| GTWDSSLRAVV | 222 | 41554 | 12 | 5890 | 504 |
| GTWDSGLSAGV | 222 | 37642 | 12 | 6122 | 487 |
| GAWDSSLSAGV | 222 | 35882 | 12 | 8303 | 407 |
| GTWDRSLSAVV | 222 | 31235 | 12 | 2689 | 762 |
| QSYDSSLSGGV | 222 | 30858 | 12 | 13372 | 280 |
| GTWDSSLNAGV | 222 | 29122 | 12 | 1476 | 1040 |
| GTWDSRLSAGV | 222 | 26248 | 12 | 2905 | 733 |
| GTWDRSLSAGV | 222 | 23871 | 12 | 2066 | 870 |
| QSYDSSLSGAV | 222 | 21879 | 12 | 17026 | 238 |
| GTWDSSLSAGG | 222 | 20912 | 12 | 5451 | 532 |
| GSWDSSLSAGV | 222 | 16501 | 12 | 4389 | 605 |
| GTWDSRLSAVV | 222 | 16331 | 12 | 3763 | 647 |
| GTWDSSLGAGV | 222 | 15684 | 12 | 6008 | 494 |
| GSWDSSLSAVV | 222 | 13998 | 12 | 5736 | 514 |
| VTWDSSLSAGV | 222 | 13890 | 12 | 1152 | 1177 |
| QSYDSSLRGSV | 222 | 12406 | 12 | 3042 | 721 |
| GTWDSSLSAGA | 222 | 10881 | 12 | 7208 | 446 |
| GTWGSSLSAGV | 222 | 9534 | 12 | 7748 | 429 |
| QSYDSSLGGSV | 222 | 9188 | 12 | 3552 | 673 |
| GTWGSSLSAVV | 222 | 8825 | 12 | 9863 | 353 |
| GTRDSSLSAVV | 222 | 8319 | 12 | 7057 | 452 |
| QSYDSGLSGSV | 222 | 7643 | 12 | 3184 | 705 |
| GSYTSSSTLV | 222 | 7633 | 12 | 9135 | 378 |
| GTRDSSLSAGV | 222 | 7393 | 12 | 5342 | 543 |
| GTWDSSPSAGV | 222 | 6757 | 12 | 5042 | 565 |
| GTWDSSPSAVV | 222 | 6121 | 12 | 6465 | 472 |
| RTWDSSLSAGV | 222 | 5674 | 12 | 3656 | 662 |
| GT*DSSLSAGV | 222 | 5195 | 12 | 4499 | 592 |
| GTWDSSLCAVV | 222 | 4936 | 12 | 3956 | 633 |
| GTWDSCLSAVV | 222 | 4896 | 12 | 3885 | 635 |
| GTWDSSLCAGV | 222 | 4488 | 12 | 2689 | 761 |
| GTWDSCLSAGV | 222 | 4260 | 12 | 2970 | 729 |
| GTWVSSLSAVV | 222 | 3976 | 12 | 3447 | 679 |
| GTWVSSLSAGV | 222 | 3611 | 12 | 2808 | 750 |
| GTCDSSLSAGV | 222 | 2722 | 12 | 2530 | 794 |
| QVWDSSSDHWV | 221 | 463184 | 12 | 398316 | 23 |
| AAWDDSLNGWV | 221 | 434855 | 12 | 460547 | 20 |
| GTWDSSLSVGV | 221 | 262755 | 12 | 7110 | 448 |

TRA:

| | Adults | Adult | | | |
|---|---|---|---|---|---|
| | with | total | CB with | CB total | |
| CDR3 | CDR3 | reads | CDR3 | reads | CB rank |
| * | 222 | 31630222 | 12 | 5706530 | 0 |
| AVRDSNYQLI | 222 | 2484161 | 12 | 13985 | 63 |
| AVMDSNYQLI | 222 | 2419871 | 12 | 16827 | 39 |
| AVLDSNYQLI | 222 | 974620 | 12 | 4757 | 384 |
| AVKDSNYQLI | 222 | 637149 | 12 | 6050 | 253 |
| AVVDSNYQLI | 222 | 438460 | 12 | 1764 | 1351 |
| AVTDSNYQLI | 222 | 437348 | 12 | 2877 | 815 |
| AVIDSNYQLI | 222 | 206157 | 11 | 592 | 4186 |
| AVNTGGFKTI | 222 | 198140 | 12 | 67618 | 2 |
| AVNQAGTALI | 222 | 173459 | 12 | 53821 | 4 |
| AVNTGFQKLV | 222 | 126814 | 12 | 52553 | 5 |
| AVNDYKLS | 222 | 125653 | 12 | 57875 | 3 |
| AVNSGGYQKVT | 222 | 121406 | 12 | 52104 | 6 |
| AVTSGTYKYI | 222 | 114374 | 12 | 15156 | 49 |
| AVDSNYQLI | 222 | 99802 | 12 | 23035 | 21 |
| AVNTNAGKST | 222 | 99401 | 12 | 41151 | 7 |
| AVNRDDKII | 222 | 98854 | 12 | 28598 | 12 |
| AVNTGNQFY | 222 | 98561 | 12 | 26839 | 15 |
| AVRDDKII | 222 | 98215 | 12 | 15520 | 42 |
| AVGGSQGNLI | 222 | 87944 | 12 | 13817 | 64 |
| AVNSGGSNYKLT | 222 | 87485 | 12 | 16633 | 40 |
| AVSGSARQLT | 222 | 84917 | 12 | 20928 | 26 |
| AVGDSNYQLI | 222 | 83755 | 11 | 4125 | 2172 |
| AVDTGRRALT | 222 | 79774 | 12 | 28633 | 11 |
| AVNNAGNMLT | 222 | 76805 | 12 | 20421 | 28 |
| AVNYGGSQGNLI | 222 | 76167 | 12 | 20567 | 27 |
| AVYTGGFKTI | 222 | 71325 | 12 | 15243 | 46 |
| AASNDYKLS | 222 | 71143 | 12 | 21842 | 24 |
| AVNSGYSTLT | 222 | 69962 | 12 | 27663 | 13 |
| AANQAGTALI | 222 | 67275 | 12 | 12401 | 80 |
| AASGGSNYKLT | 222 | 67247 | 12 | 7443 | 183 |
| AVSGGSYIPT | 222 | 65402 | 12 | 19425 | 32 |
| ALMDSNYQLI | 222 | 65061 | 11 | 2670 | 2312 |
| ALNTGGFKTI | 222 | 64776 | 12 | 17696 | 38 |
| AANFGNEKLT | 222 | 62582 | 12 | 12435 | 78 |
| AASTSGTYKYI | 222 | 61388 | 12 | 15306 | 45 |
| AVNTDKLI | 222 | 61383 | 12 | 16320 | 41 |
| AVTGNQFY | 222 | 57859 | 12 | 26234 | 16 |
| AVLNQAGTALI | 222 | 56941 | 12 | 14636 | 54 |
| AASGGSYIPT | 222 | 55206 | 12 | 19916 | 29 |
| AVKAAGNKLT | 222 | 54908 | 12 | 23078 | 19 |
| AVNAGNNRKLI | 222 | 54337 | 12 | 12187 | 81 |
| AVSGGSNYKLT | 222 | 54001 | 12 | 10310 | 100 |
| AANAGGTSYGKLT | 222 | 53565 | 12 | 6163 | 245 |
| ALNDYKLS | 222 | 52490 | 12 | 14163 | 60 |
| AVSGGYQKVT | 222 | 52480 | 12 | 18706 | 34 |
| AVDTGGFKTI | 222 | 51993 | 12 | 15177 | 48 |
| AVRNTGGFKTI | 222 | 50632 | 12 | 19796 | 31 |
| AVRSNDYKLS | 222 | 45064 | 12 | 14087 | 61 |
| AVSSNDYKLS | 222 | 44704 | 12 | 12986 | 72 |
| AVTGTASKLT | 222 | 44385 | 12 | 8110 | 161 |
| AVNTGTASKLT | 222 | 44042 | 12 | 8547 | 147 |
| AVAGGTSYGKLT | 222 | 42600 | 12 | 4880 | 370 |
| AVTTSGTYKYI | 222 | 42488 | 12 | 9417 | 118 |
| AGGGSQGNLI | 222 | 42001 | 12 | 6018 | 254 |
| AVHTGGFKTI | 222 | 41198 | 12 | 9301 | 121 |
| AVYNTDKLI | 222 | 39547 | 12 | 8052 | 165 |
| VVNTGNQFY | 222 | 38222 | 12 | 1828 | 1316 |
| AVSSGSARQLT | 222 | 37918 | 12 | 9037 | 128 |
| AARDSNYQLI | 222 | 36714 | 12 | 2595 | 921 |
| AANNAGNMLT | 222 | 36366 | 12 | 6348 | 236 |
| AVSNFGNEKLT | 222 | 35927 | 12 | 8851 | 134 |
| AVSDTGGFKTI | 222 | 34204 | 12 | 15053 | 50 |
| AVDRGSTLGRLY | 222 | 33576 | 12 | 11694 | 87 |
| AASSGSARQLT | 222 | 32033 | 12 | 7516 | 182 |
| ALGGSQGNLI | 222 | 31359 | 12 | 2995 | 775 |
| AASSGGYQKVT | 222 | 30644 | 12 | 6639 | 217 |
| AVSNTGGFKTI | 222 | 28991 | 12 | 6433 | 226 |
| AASAGGTSYGKLT | 222 | 27852 | 12 | 3493 | 635 |
| AVSAGGTSYGKLT | 222 | 27808 | 12 | 2730 | 870 |
| AVMDSSYKLI | 221 | 618800 | 12 | 29126 | 10 |
| AAMDSNYQLI | 221 | 467502 | 12 | 3852 | 541 |
| AVSDSNYQLI | 221 | 438680 | 12 | 5139 | 337 |
| AALDSNYQLI | 221 | 307036 | 11 | 2192 | 2482 |
| AVEDSNYQLI | 221 | 127093 | 12 | 3511 | 630 |
| AASDSNYQLI | 221 | 93800 | 10 | 2592 | 4709 |
| AVSNDYKLS | 221 | 92075 | 12 | 22888 | 22 |
| AASKGGSYIPT | 221 | 84938 | 12 | 17748 | 37 |
| AVPNQAGTALI | 221 | 83407 | 12 | 13633 | 67 |
| AVNFGNEKLT | 221 | 78840 | 12 | 13310 | 70 |
| AVRDNYGQNFV | 221 | 76903 | 12 | 21076 | 25 |
| AAYTGGFKTI | 221 | 74430 | 12 | 5396 | 311 |
| AVNAGGTSYGKLT | 221 | 74088 | 12 | 6634 | 218 |
| ALSGGSNYKLT | 221 | 61394 | 12 | 3392 | 668 |
| ALYNFNKFY | 221 | 57631 | 12 | 15469 | 43 |
| AVYSSASKII | 221 | 55926 | 12 | 14972 | 51 |
| AGGTSYGKLT | 221 | 55857 | 12 | 7436 | 184 |
| AVASGGSYIPT | 221 | 54756 | 12 | 15231 | 47 |
| VVNTNAGKST | 221 | 54397 | 12 | 24281 | 17 |
| AVNSGNTPLV | 221 | 53299 | 12 | 19818 | 30 |
| AVSNQAGTALI | 221 | 52716 | 12 | 12762 | 73 |
| AENSGGSNYKLT | 221 | 52328 | 12 | 3155 | 737 |
| AVRGSQGNLI | 221 | 51440 | 12 | 10808 | 95 |
| AASNQAGTALI | 221 | 50554 | 12 | 12585 | 77 |
| AAGGSQGNLI | 221 | 49041 | 12 | 5279 | 320 |
| AVQTGANNLF | 221 | 48165 | 12 | 9895 | 102 |
| AVYNFNKFY | 221 | 46850 | 12 | 23061 | 20 |
| AVKTSYDKVI | 221 | 46512 | 12 | 6139 | 247 |
| AVFTGGGNKLT | 221 | 46491 | 12 | 8507 | 150 |

TRB:

| | Adults | Adult | | | |
|---|---|---|---|---|---|
| | with | total | CB with | CB total | |
| CDR3 | CDR3 | reads | CDR3 | reads | CB rank |
| * | 222 | 3232641 | 30 | 10045379 | 0 |
| ASSLNTEAF | 212 | 13823 | 30 | 41459 | 19 |
| ASSLGGNTEAF | 209 | 78580 | 30 | 29808 | 42 |
| ASSPSTDTQY | 208 | 11606 | 30 | 38520 | 22 |
| ASSLTDTQY | 207 | 12809 | 30 | 50876 | 11 |
| ASSLGQNTEAF | 206 | 10690 | 30 | 93056 | 1 |
| ASSLSYEQY | 205 | 12321 | 30 | 46016 | 13 |
| ASSLQGNTEAF | 205 | 9290 | 30 | 30158 | 39 |
| ASSLGGTEAF | 203 | 10995 | 30 | 58963 | 7 |
| ASSLGSNQPQH | 202 | 9827 | 29 | 21250 | 203 |
| ASSLDSNQPQH | 201 | 9697 | 30 | 35364 | 28 |
| ASSLGGNQPQH | 201 | 9444 | 30 | 32531 | 33 |
| ASSLQETQY | 200 | 13863 | 29 | 36845 | 183 |
| ASSLGYEQY | 199 | 10688 | 30 | 82685 | 3 |
| ASSLGNTEAF | 198 | 10130 | 30 | 62782 | 6 |
| ASSLGRNTEAF | 198 | 8136 | 30 | 14092 | 101 |
| ASSLGLNTEAF | 198 | 7059 | 30 | 14660 | 97 |
| ASSSTDTQY | 197 | 12875 | 30 | 33841 | 32 |
| ASSLSTDTQY | 197 | 12688 | 30 | 34516 | 29 |
| ASSPSYEQY | 196 | 22288 | 30 | 26730 | 51 |
| ASSLTGNTEAF | 196 | 9003 | 30 | 43428 | 17 |
| ASSSSYEQY | 193 | 12057 | 30 | 39636 | 20 |
| ASSLGNQPQH | 193 | 8119 | 29 | 29007 | 187 |
| ASSLDRNTEAF | 193 | 7350 | 30 | 18871 | 75 |
| ASSLGTDTQY | 192 | 8503 | 30 | 36873 | 25 |
| ASSFTDTQY | 192 | 7701 | 30 | 51198 | 10 |
| ASSLGGYEQY | 192 | 7268 | 30 | 44934 | 15 |
| ASSLGTEAF | 191 | 9278 | 30 | 31798 | 36 |
| ASSLGSYEQY | 191 | 6203 | 30 | 21315 | 69 |
| ASSLYNEQF | 190 | 9347 | 29 | 31384 | 184 |
| ASSLSSYEQY | 190 | 8112 | 30 | 24961 | 55 |
| ASSSQETQY | 190 | 7476 | 30 | 26896 | 50 |
| ASSSYNEQF | 189 | 9418 | 28 | 24165 | 478 |
| ASSLTVNTEAF | 189 | 5874 | 30 | 12387 | 108 |
| ASSLGETQY | 187 | 8315 | 30 | 28449 | 45 |
| ASSPQETQY | 186 | 8112 | 30 | 19624 | 72 |
| ASSLGGSYEQY | 185 | 5108 | 29 | 21499 | 201 |
| ASSLEETQY | 184 | 8580 | 30 | 45458 | 14 |
| ASSYSYEQY | 184 | 7369 | 30 | 31559 | 37 |
| ASSQETQY | 184 | 7316 | 30 | 50375 | 12 |
| ASSLSNQPQH | 184 | 6955 | 29 | 11332 | 273 |
| ASSLGGTDTQY | 184 | 6508 | 30 | 41732 | 18 |
| ASSLGVNTEAF | 183 | 5966 | 30 | 14681 | 96 |
| ASSLGQGNQPQH | 183 | 5711 | 29 | 36855 | 182 |
| ASSLGPNTEAF | 181 | 12069 | 28 | 7027 | 657 |
| ASSLADTQY | 181 | 7614 | 30 | 69188 | 5 |
| ASSLGGGTEAF | 180 | 5012 | 30 | 12047 | 109 |
| ASSLAGNTEAF | 179 | 5619 | 30 | 20132 | 71 |
| ASSLGDTQY | 178 | 6761 | 30 | 25457 | 54 |
| ASSSSTDTQY | 178 | 6751 | 29 | 20877 | 204 |
| ASSLTGGTEAF | 178 | 5589 | 30 | 29963 | 40 |
| ASSPSSYEQY | 177 | 7892 | 29 | 21349 | 202 |
| ASSLGQNYGYT | 177 | 7841 | 30 | 24043 | 58 |
| ASSFSTDTQY | 177 | 6025 | 29 | 25647 | 192 |
| ASSLGQGNTEAF | 177 | 5516 | 27 | 17849 | 941 |
| ASSLGGSNQPQH | 176 | 5953 | 30 | 17971 | 79 |
| ASSPGQGNQPQH | 175 | 4817 | 30 | 14469 | 99 |
| ASSLGGGNQPQH | 175 | 4399 | 29 | 11008 | 279 |
| ASSLAGNQPQH | 174 | 6410 | 28 | 10609 | 564 |
| ASSRNTEAF | 174 | 6391 | 29 | 9856 | 295 |
| ASSLRGNTEAF | 174 | 4979 | 29 | 12454 | 256 |
| ASSFSYEQY | 173 | 10491 | 29 | 28905 | 188 |
| ASSQGYEQY | 173 | 6693 | 30 | 31387 | 38 |
| ASSLQGNQPQH | 173 | 6309 | 30 | 52206 | 9 |
| ASSLAGGTDTQY | 173 | 5647 | 30 | 88853 | 2 |
| ASSLLNTEAF | 173 | 5533 | 27 | 8387 | 1022 |
| ASSRDSNQPQH | 173 | 5055 | 29 | 16493 | 221 |
| ASSLGGSTDTQY | 173 | 4748 | 30 | 16234 | 89 |
| ASSFQETQY | 172 | 8095 | 29 | 30678 | 185 |
| ASSLMNTEAF | 171 | 8072 | 26 | 10161 | 1567 |
| ASSLGGYGYT | 171 | 5920 | 29 | 50056 | 179 |
| ASSLYSNQPQH | 170 | 7229 | 27 | 15394 | 950 |
| ASSPGQNTEAF | 170 | 5238 | 30 | 18604 | 76 |
| ASSPDRNTEAF | 170 | 4900 | 29 | 5976 | 394 |
| ASSLVGNTEAF | 170 | 4820 | 28 | 7707 | 633 |
| ASSLGGSSYEQY | 169 | 8757 | 28 | 13691 | 517 |
| ASSPPSTDTQY | 169 | 6162 | 29 | 16783 | 219 |
| ASSRQGNTEAF | 169 | 4929 | 30 | 8618 | 136 |
| ASSLGQGYGYT | 168 | 4742 | 29 | 23156 | 197 |
| ASSLGSSYEQY | 168 | 4545 | 28 | 15272 | 510 |
| ASSRTDTQY | 167 | 9327 | 30 | 23466 | 61 |
| ASSQDSNQPQH | 167 | 6131 | 29 | 20395 | 207 |
| ASSLTGNQPQH | 167 | 5567 | 30 | 17702 | 80 |
| ASSPGQGYEQY | 167 | 5302 | 28 | 16918 | 498 |
| ASSLGSTDTQY | 167 | 5111 | 29 | 19179 | 212 |
| ASSLEGNTEAF | 167 | 5038 | 29 | 8260 | 333 |
| ASSLGQLNTEAF | 167 | 4706 | 26 | 5229 | 1742 |
| ASSLGQGYEQY | 166 | 7473 | 30 | 37663 | 23 |
| ASSYTDTQY | 166 | 5723 | 30 | 24354 | 56 |
| ASSQGLNTEAF | 166 | 5404 | 30 | 8352 | 140 |
| ASSLDSYEQY | 166 | 5026 | 30 | 34277 | 31 |
| ASSLGGQPQH | 166 | 3130 | 30 | 13357 | 103 |
| ASSLTENTEAF | 165 | 6466 | 29 | 4460 | 432 |
| ASSLNSNQPQH | 165 | 6102 | 29 | 7244 | 367 |
| ASSLSGNTEAF | 165 | 4195 | 28 | 10208 | 577 |
| ASSLGQGAYEQY | 164 | 4663 | 28 | 21213 | 480 |
| ASSLEGNQPQH | 163 | 6241 | 28 | 6889 | 662 |
| ASSLKETQY | 163 | 6186 | 30 | 16445 | 87 |
| ASSLAGGTEAF | 163 | 5934 | 30 | 17572 | 81 |

TRD:

| | Adults | Adult | | | |
|---|---|---|---|---|---|
| | with | total | CB with | CB total | |
| CDR3 | CDR3 | reads | CDR3 | reads | CB rank |
| * | 222 | 542467 | 30 | 227046 | 0 |
| ACDTLGDTDKLI | 141 | 29665 | 16 | 1790 | 53 |
| ACDTVGGYTDKLI | 110 | 15252 | 8 | 250 | 392 |
| ACDTVGDTDKLI | 95 | 9152 | 18 | 3924 | 34 |
| ACDRLGDTDKLI | 86 | 4481 | 3 | 64 | 2272 |
| ACDILGDTDKLI | 83 | 6717 | 22 | 11178 | 5 |
| ACDTLLGDTDKLI | 83 | 6272 | 5 | 641 | 822 |
| ACDPLGDTDKLI | 79 | 9247 | 4 | 157 | 1216 |
| ACDPLLGDTDKLI | 79 | 5313 | 3 | 108 | 2033 |
| ACDTVGGTDKLI | 79 | 4649 | 6 | 103 | 681 |
| ACDVLGDTDKLI | 76 | 5983 | 22 | 9191 | 6 |
| ACDTLGGTDKLI | 73 | 2992 | 3 | 4 | 2952 |
| ACDSLGDTDKLI | 69 | 2796 | 4 | 7 | 1527 |
| ACDLLGDTDKLI | 66 | 4529 | 16 | 2082 | 51 |
| ACDALGDTDKLI | 61 | 1839 | 11 | 67 | 220 |
| ACDTVGEYTDKLI | 56 | 9482 | 3 | 5 | 2725 |
| ACDTAGGSSWDTRQMF | 55 | 2546 | 14 | 737 | 100 |
| ACDTVGGSTDKLI | 54 | 2614 | | | |
| ACDSLLGDTDKLI | 52 | 4681 | 5 | 63 | 960 |
| ACDTLGDTSDKLI | 52 | 2673 | | | |
| ACDTVGGNTDKLI | 51 | 5007 | 2 | 26 | 8860 |
| ACDTLGYTDKLI | 51 | 3930 | 5 | 281 | 841 |
| ACDTVLGDTSSWDTRQMF | 51 | 2342 | 20 | 1380 | 16 |
| ACDTVGTYTDKLI | 50 | 3525 | | | |
| ACDTVLGDSSWDTRQMF | 50 | 2193 | 19 | 2355 | 27 |
| ACDTVGSYTDKLI | 49 | 3478 | 1 | 44 | 37807 |
| ACDALLGDTDKLI | 49 | 1466 | 3 | 118 | 1995 |
| ACDTVGAYTDKLI | 48 | 4388 | 1 | 24 | 69120 |
| ACDKLGDTDKLI | 48 | 3339 | 10 | 513 | 252 |
| ACDTVGGHTDKLI | 48 | 2831 | | | |
| ACDTVGGSDKLI | 47 | 3635 | 2 | 36 | 7865 |
| ACDTLGDADKLI | 47 | 1034 | 1 | 7 | 120797 |
| ACDTLGGYTDKLI | 46 | 76073 | 2 | 126 | 3898 |
| ACDTLGDSDKLI | 46 | 5325 | 3 | 3 | 3020 |
| ACDTVGYTDKLI | 46 | 1146 | 12 | 409 | 172 |
| ACDTVGVYTDKLI | 45 | 2536 | 2 | 87 | 4619 |
| ACGTLGDTDKLI | 45 | 120 | 4 | 12 | 1457 |
| ACDTVGGSYTDKLI | 44 | 2647 | 4 | 348 | 1154 |
| ACDTLGAYTDKLI | 43 | 2780 | 1 | 1 | 255402 |
| ACDTVGGRTDKLI | 42 | 2901 | | | |
| ACDTLGDTGTDKLI | 42 | 1600 | | | |
| ACDTGGYGSWDTRQMF | 42 | 1005 | 18 | 974 | 37 |
| ARDTLGDTDKLI | 42 | 98 | 3 | 3 | 3281 |
| ACDTVLGDTRYTDKLI | 41 | 1762 | 1 | 11 | 105467 |
| ACDTLGVTDKLI | 40 | 1400 | 1 | 2 | 166231 |
| ACDTVGGYADKLI | 40 | 872 | 5 | 132 | 912 |
| ACDTVGGDTDKLI | 39 | 1262 | 1 | 1 | 191717 |
| ACDTLGETDKLI | 39 | 1217 | | | |
| ACDTLGDTDKLT | 39 | 99 | 2 | 2 | 11117 |
| ACDTLGDTDKPI | 39 | 89 | 3 | 4 | 2884 |
| ACDTLGTYTDKLI | 38 | 7485 | 1 | 1 | 443491 |
| ACDTLGDTRTDKLI | 38 | 5111 | 1 | 180 | 14010 |
| ACDTVLGDTSWDTRQMF | 38 | 1484 | 10 | 409 | 259 |
| ACDTLGEYTDKLI | 38 | 1299 | 2 | 49 | 6656 |
| ACDTLGVYTDKLI | 37 | 3448 | 2 | 86 | 4643 |
| ACDTLGANTDKLI | 37 | 1328 | 1 | 71 | 21758 |
| ACDSVGGYTDKLI | 37 | 990 | 2 | 2 | 11889 |
| ACDTLGDTADKLI | 36 | 7244 | | | |
| ACDTWGNTDKLI | 36 | 2252 | 6 | 502 | 615 |
| ACDTLGDTYTDKLI | 36 | 1021 | 5 | 179 | 884 |
| ACDTVGENTDKLI | 36 | 888 | | | |
| ACDTLGNTDKLI | 35 | 3570 | 3 | 6 | 2698 |
| ACDTLLGDTYTDKLI | 35 | 2449 | 2 | 23 | 9120 |
| ACDTWGTDKLI | 35 | 1744 | 11 | 1964 | 198 |
| ACDTVGGLYTDKLI | 35 | 1581 | 3 | 74 | 2205 |
| ACDTVGGFTDKLI | 34 | 1021 | | | |
| ACDTLGGNTDKLI | 34 | 976 | | | |
| ACDTVGPYTDKLI | 34 | 964 | | | |
| ACDTAGGSWDTRQMF | 33 | 4457 | 15 | 2377 | 68 |
| ACDPLGDYTDKLI | 33 | 3116 | | | |
| ACDTVGGPYTDKLI | 33 | 2733 | 3 | 76 | 2191 |
| ACDTLGENTDKLI | 33 | 2672 | | | |
| ACDTVGLYTDKLI | 33 | 1847 | | | |
| ACDTLGDTDTDKLI | 33 | 1312 | 2 | 90 | 4524 |
| ACDTWGDTDKLI | 33 | 719 | 5 | 428 | 827 |
| ACDTVGVTDKLI | 33 | 285 | 5 | 7 | 1049 |
| ACDTLGDTVKLI | 33 | 43 | 1 | 1 | 265200 |
| ACDTVGGTYTDKLI | 32 | 9193 | | | |
| ACDTVGANTDKLI | 32 | 709 | | | |
| ACDTLGDSYTDKLI | 31 | 4062 | | | |
| ACDTLLGDTRYTDKLI | 31 | 3983 | 2 | 52 | 6400 |
| ACDTVLGTDKLI | 31 | 1731 | 2 | 98 | 4323 |
| ACDTLGGPYTDKLI | 31 | 1271 | 1 | 259 | 13410 |
| ACDTVGGLTDKLI | 31 | 479 | | | |
| ACDTLGDTGKLI | 31 | 270 | | | |
| ACDTVLGDTRSWDTRQMF | 30 | 7074 | 11 | 989 | 201 |
| ACDTLGDPYTDKLI | 30 | 4955 | | | |
| ACDTLGATDKLI | 30 | 1756 | | | |
| ACDTVGGGTDKLI | 30 | 1720 | | | |
| ACDTVLGDTWDTRQMF | 30 | 1163 | 19 | 5546 | 23 |
| ACDTLGDTRDKLI | 30 | 1076 | | | |
| ACDTTGGSWDTRQMF | 30 | 647 | 12 | 328 | 173 |
| ACDTVGGRYTDKLI | 30 | 510 | 1 | 1 | 312790 |
| ACDPLGGYTDKLI | 30 | 468 | 1 | 13 | 100760 |
| ACDTVGGGYTDKLI | 29 | 7000 | 4 | 92 | 1293 |
| ACDTWGYTDKLI | 29 | 5325 | 19 | 2808 | 26 |
| ACDTGGYSSWDTRQMF | 29 | 1743 | 18 | 2948 | 35 |
| ACDTVGDSDKLI | 29 | 1306 | 3 | 9 | 2616 |
| ACDTLLGDTTDKLI | 29 | 1116 | | | |

TRG:

| | Adults | Adult | | | |
|---|---|---|---|---|---|
| | with | total | CB with | CB total | |
| CDR3 | CDR3 | reads | CDR3 | reads | CB rank |
| * | 222 | 10972664 | 12 | 527036 | 0 |
| ALWEVQELGKKIKV | 220 | 930787 | 12 | 19295 | 1 |
| ALWEVRELGKKIKV | 219 | 325307 | 10 | 2791 | 107 |
| ALWEVLELGKKIKV | 217 | 143247 | 8 | 708 | 298 |
| ATWDGYYKKL | 217 | 62279 | 12 | 9663 | 4 |
| ATWDGNYYKKL | 216 | 159487 | 12 | 5790 | 5 |
| ALWEVQEFGKKIKV | 216 | 22110 | 10 | 249 | 176 |
| ALWEGQELGKKIKV | 215 | 52599 | 9 | 694 | 205 |
| ALWEAQELGKKIKV | 214 | 177886 | 9 | 957 | 190 |
| ATWDYYKKL | 214 | 45235 | 12 | 10231 | 3 |
| ATWDGPYYKKL | 214 | 36592 | 12 | 5403 | 6 |
| ALWDVQELGKKIKV | 214 | 15436 | 9 | 161 | 267 |
| ALWEVQELCKKIKV | 214 | 7557 | 8 | 48 | 435 |
| ALWEVQELGKIIKV | 213 | 15574 | 4 | 16 | 2230 |
| ALGEVQELGKKIKV | 213 | 12633 | 10 | 123 | 183 |
| ALWEVKELGKKIKV | 212 | 96792 | 9 | 1205 | 186 |
| ATWDYKKL | 212 | 40158 | 12 | 5064 | 7 |
| ALWEVGELGKKIKV | 211 | 91481 | 9 | 629 | 208 |
| ALWEEQELGKKIKV | 211 | 58792 | 6 | 267 | 744 |
| ATWDGLYYKKL | 211 | 49650 | 12 | 3521 | 12 |
| ALWEVQELVKKIKV | 211 | 8768 | 7 | 67 | 638 |
| ALWEVHELGKKIKV | 209 | 123803 | 7 | 699 | 463 |
| ATWDGRYKKL | 209 | 40700 | 12 | 4455 | 9 |
| ALWEVEELGKKIKV | 208 | 77355 | 8 | 519 | 310 |
| ALWVVQELGKKIKV | 208 | 5707 | 5 | 58 | 1369 |
| ATWDKKL | 206 | 24521 | 12 | 3367 | 13 |
| ALWEEELGKKIKV | 205 | 94022 | 8 | 1336 | 289 |
| ATWDGPNYYKKL | 205 | 24307 | 12 | 4077 | 10 |
| ATWDGRYYKKL | 204 | 26665 | 12 | 2958 | 14 |
| ATWDRNYYKKL | 204 | 22662 | 11 | 2540 | 52 |
| ALWEGRELGKKIKV | 204 | 8113 | 7 | 101 | 627 |
| GLWEVQELGKKIKV | 204 | 4567 | 7 | 26 | 661 |
| ALWEVREFGKKIKV | 203 | 5839 | 7 | 26 | 660 |
| ALWEDQELGKKIKV | 202 | 80424 | 7 | 177 | 566 |
| ATWDGPGYKKL | 202 | 29370 | 11 | 3095 | 50 |
| ALWEVQGLGKKIKV | 202 | 9169 | 7 | 228 | 537 |
| ALWEVQEVGKKIKV | 202 | 3725 | 8 | 42 | 438 |
| ATWDGPYKKL | 201 | 25216 | 12 | 3716 | 11 |
| ALWEQELGKKIKV | 200 | 68666 | 10 | 4073 | 106 |
| ALWELQELGKKIKV | 200 | 40369 | 7 | 609 | 468 |
| ALWEVQVLGKKIKV | 200 | 7298 | 6 | 29 | 980 |
| ALCEVQELGKKIKV | 200 | 3696 | 8 | 47 | 436 |
| ALREVQELGKKIKV | 200 | 3325 | 9 | 66 | 279 |
| ALWEVRYKKL | 199 | 75533 | 10 | 1666 | 110 |
| ALWETQELGKKIKV | 199 | 68450 | 8 | 460 | 319 |
| ALWEVNYYKKL | 199 | 35940 | 12 | 2438 | 17 |
| ATWDGLNYYKKL | 199 | 31905 | 11 | 1100 | 71 |
| ALWESQELGKKIKV | 199 | 30392 | 5 | 236 | 1095 |
| ATWDSYYKKL | 199 | 14283 | 11 | 1996 | 53 |
| ALWEVELGKKIKV | 198 | 55241 | 9 | 2280 | 184 |
| ALWEVSELGKKIKV | 198 | 39221 | 7 | 133 | 603 |
| ALWGVQELGKKIKV | 198 | 3583 | 8 | 38 | 440 |
| ATWDGYKKL | 197 | 39035 | 12 | 4690 | 8 |
| ATWDGHYYKKL | 197 | 19135 | 10 | 1983 | 108 |
| ALWEVQELGKKIRV | 197 | 2380 | 6 | 43 | 965 |
| ALWEVQELGKKINV | 197 | 2011 | 8 | 53 | 433 |
| ALWEPQELGKKIKV | 196 | 47093 | 10 | 854 | 129 |
| ALWEVQ*LGKKIKV | 196 | 2370 | 6 | 35 | 972 |
| ALWEVPELGKKIKV | 195 | 51740 | 7 | 174 | 572 |
| ATWDGKKL | 195 | 37653 | 11 | 1970 | 54 |
| ATWDRPYYKKL | 195 | 16983 | 12 | 2491 | 16 |
| ALWEVRELGKIIKV | 195 | 5816 | 2 | 2 | 9214 |
| ALGEVRELGKKIKV | 195 | 5102 | 6 | 19 | 997 |
| SLWEVQELGKKIKV | 195 | 2831 | 5 | 14 | 1474 |
| ALLEVQELGKKIKV | 195 | 2794 | 7 | 22 | 665 |
| ALVV*VQELGKKIKV | 195 | 2612 | 6 | 21 | 991 |
| ALWEVVELGKKIKV | 194 | 30873 | 4 | 91 | 1828 |
| ATWDGHYKKL | 193 | 28319 | 12 | 2727 | 15 |
| ATWDTTGWFKI | 193 | 21628 | 12 | 12462 | 2 |
| ATWDGLYKKL | 193 | 13754 | 12 | 1520 | 28 |
| ALWEVELGKKIKV | 193 | 2530 | 6 | 33 | 974 |
| ASWEVQELGKKIKV | 192 | 2233 | 8 | 54 | 432 |
| ALWEARELGKKIKV | 190 | 15909 | 5 | 111 | 1236 |
| AVWEVQELGKKIKV | 190 | 2346 | 3 | 8 | 3698 |
| ALWEVQDLGKKIKV | 190 | 2344 | 7 | 23 | 664 |
| ALWEVYYKKL | 189 | 26888 | 12 | 2091 | 20 |
| ATWDRYYKKL | 189 | 13497 | 11 | 1351 | 64 |
| ATWDRRYYKKL | 189 | 9538 | 11 | 1614 | 58 |
| ATWDNYYKKL | 189 | 7036 | 10 | 1414 | 113 |
| ALWEVLEFGKKIKV | 189 | 3369 | 6 | 9 | 1035 |
| ALWEVQELGKKVKV | 189 | 2471 | 9 | 52 | 281 |
| ALWEVQELRKKIKV | 189 | 2257 | 3 | 6 | 3733 |
| ALWEVQELGKKIEV | 189 | 1992 | 7 | 45 | 648 |
| ALWEGELGKKIKV | 188 | 30065 | 7 | 474 | 472 |
| ATWDRRYKKL | 188 | 25624 | 10 | 1518 | 111 |
| ATWDGYYYKKL | 188 | 10589 | 11 | 1072 | 73 |
| ALWEVRELVKKIKV | 188 | 3174 | 4 | 5 | 2487 |
| ALWEVRELCKKIKV | 188 | 2671 | 4 | 8 | 2308 |
| ALWEVQE*GKKIKV | 188 | 2239 | 5 | 10 | 1509 |
| ATWENYYKKL | 187 | 61856 | 12 | 1587 | 25 |
| ATWDGPGYYKKL | 187 | 17680 | 12 | 2103 | 19 |
| ATWDRLYYKKL | 187 | 15783 | 12 | 1286 | 34 |
| ATWDVYYKKL | 187 | 10452 | 11 | 966 | 78 |
| ATWDGDYYKKL | 187 | 9735 | 11 | 1871 | 56 |
| ALWEAQEFGKKIKV | 187 | 4268 | 5 | 10 | 1513 |
| ALWDVRELGKKIKV | 187 | 3809 | 5 | 11 | 1498 |
| ATWDRPGYKKL | 186 | 6708 | 11 | 1304 | 66 |
| ALWEGGELGKKIKV | 186 | 4428 | 7 | 58 | 645 |
| ALWEKELGKKIKV | 185 | 22273 | 5 | 215 | 1104 |

### Diversity Index

The third index disclosed herein is referred to as the diversity index. This method uses the difference between the level of immune cell diversity generally seen in a normal, healthy individual and the generally lower level of diversity seen in an individual who has one or more disease conditions as a diagnostic indicator of the presence of a normal or abnormal immune status. In one aspect of the invention, the diversity level is referred to as the D50, with D50 being defined as the minimum percentage of distinct CDR3s accounting for at least half of the total CDR3s in a population or subpopulation of immune system cells. The third complementarity-determining region (CDR3) being a region whose nucleotide sequence is unique to each T or B cell clone, the higher the number, the greater the level of diversity. D50 may be described as follows. Where the “significant percentage” of the total number cells is fifty percent (50%), the diversity index (D50) may also be defined as a measure of the diversity of an immune repertoire of J individual cells (the total number of CDR3s) composed of S distinct CDR3s in a ranked dominance configuration where rᵢ is the abundance of the ith most abundant CDR3, r₁ is the abundance of the most abundant CDR3, r₂ is the abundance of the second most abundant CDR3, and so on. C is the minimum number of distinct CDR3s, amounting to 50% of the total sequencing reads. D50 therefore is given by C/S×100.

$$Assume that \underbrace{{r}_{1} \geq {r}_{2} … \geq {r}_{i} \geq {r}_{i + 1} … \geq {r}_{s}}_{S} , {\sum }_{i = 1}^{S} {r}_{i} = J$$

$$if {\sum }_{i = 1}^{C} {r}_{i} \geq J / 2$$

$$and$$

$${\sum }_{i = 1}^{C - 1} {r}_{i} \geq J / 2$$

$$D 50 = \frac{C}{S} \times 100$$

The method of the invention may be performed using the following steps for assessing the level of diversity of an immunorepertoire: (a) amplifying polynucleotides from a population of white blood cells from a human or animal subject in a reaction mix comprising target-specific nested primers to produce a set of first amplicons, at least a portion of the target-specific nested primers comprising additional nucleotides which, during amplification, serve as a template for incorporating into the first amplicons a binding site for at least one common primer; (b) transferring a portion of the first reaction mix containing the first amplicons to a second reaction mix comprising at least one common primer; (c) amplifying, using the at least one common primer, the first amplicons to produce a set of second amplicons; (d) sequencing the second amplicons to identify V(D)J rearrangement sequences in the subpopulation of white blood cells, (e) using the identified V(D)J rearrangement sequences to quantify both the total number of cells in a population of immune system cells and the total numbers of cells within each of the clonotypes identified within the population; and (f) identifying the number of clonotypes that comprise a significant percentage of a total number of cells counted within that population, wherein a normal state is characterized by the presence of a greater variety of clonotypes represented within the significant percentage of the total number of cells and an abnormal state is characterized by the presence of a lesser number of clonotypes represented within a significant percentage of the total number of cells.

It has previously been difficult to assess the immune system in a broad manner, because the number and variety of cells in a human or animal immune system is so large that sequencing of more than a small subset of cells has been almost impossible. The inventor developed a semi-quantitative PCR method (arm-PCR, described in more detail in U.S. Patent Application Publication Number 20090253183), which provides increased sensitivity and specificity over previously-available methods, while producing semi-quantitative results. It is this ability to increase specificity and sensitivity, and thereby increase the number of targets detectable within a single sample that makes the method ideal for detecting relative numbers of clonotypes of the immunorepertoire. The inventor has more recently discovered that using this sequencing method allows him to compare immunorepertoires of individual subjects, which has led to the development of the present method. The method has been used to evaluate subjects who appear normal, healthy, and asymptomatic, as well as subjects who have been diagnosed with various forms of cancer, for example, and the inventor has demonstrated that the presence of disease correlates with decreased immunorepertoire diversity, which can be readily detected using the method of the invention. This method may therefore be useful as a diagnostic indicator, much as cell counts and biochemical tests are currently used in clinical practice.

Clonotypes (i.e., clonal types) of an immunorepertoire are determined by the rearrangement of Variable(V), Diverse(D) and Joining(J) gene segments through somatic recombination in the early stages of immunoglobulin(Ig) and T cell receptor (TCR) production of the immune system. The V(D)J rearrangement can be amplified and detected from T cell receptor alpha, beta, gamma, and delta chains, as well as from immunoglobulin heavy chain (IgH) and light chains (IgK, IgL). Cells may be obtained from an individual by obtaining peripheral blood, lymphoid tissue, cancer tissue, or tissue or fluids from other organs and/or organ systems, for example. Techniques for obtaining these samples, such as blood samples, are known to those of skill in the art. “Quantifying clonotypes,” as used herein, means counting, or obtaining a reliable approximation of, the numbers of cells belonging to a particular clonotype. Cell counts may be extrapolated from the number of sequences detected by PCR amplification and sequencing.

The CDR3 region, comprising about 30-90 nucleotides, encompasses the junction of the recombined variable (V), diversity (D) and joining (J) segments of the gene. It encodes the binding specificity of the receptor and is useful as a sequence tag to identify unique V(D)J rearrangements.

Wang et al. disclosed that PCR may be used to obtain quantitative or semi-quantitative assessments of the numbers of target molecules in a specimen (Wang, M. et al., “Quantitation of mRNA by the polymerase chain reaction,” (1989) Proc. Nat'l. Acad. Sci. 86: 9717-9721). Particularly effective methods for achieving quantitative amplification have been described previously by the inventor. One such method is known as arm-PCR, which is described in United States Patent Application Publication Number 20090253183A1.

Aspects of the invention include arm-PCR amplification of CDR3 from T cells, B cells, and/or subsets of T or B cells. The term “population” of cells, as used herein, therefore encompasses what are generally referred to as either “populations” or “sub-populations” of cells. Large numbers of amplified products may then be efficiently sequenced using next-generation sequencing using platforms such as 454 or Illumina, for example. If the significant percentage that is chosen is 50%, the number may be referred to as the “D50.” D50 may then be the percent of dominant and unique T or B cell clones that account for fifty percent (50%) of the total T or B cells counted in that sample. For high-throughput sequencing, for example, the D50 may be the number of the most dominant CDR3s, among all unique CDR3s, that make up 50% of the total effective reads, where total effective reads is defined as the number of sequences with identifiable V and J gene segments which have been successfully screened through a series of error filters.

The arm-PCR method provides highly sensitive, semi-quantitative amplification of multiple polynucleotides in one reaction. The arm-PCR method may also be performed by automated methods in a closed cassette system (iCubate®, Huntsville, Ala.), which is beneficial in the present method because the repertoires of various T and B cells, for example, are so large. In the arm-PCR method, target numbers are increased in a reaction driven by DNA polymerase, which is the result of target-specific primers being introduced into the reaction. An additional result of this amplification reaction is the introduction of binding sites for common primers which will be used in a subsequent amplification by transferring a portion of the first reaction mix containing the first set of amplicons to a second reaction mix comprising common primers. “At least one common primer,” as used herein, refers to at least one primer that will bind to such a binding site, and includes pairs of primers, such as forward and reverse primers. This transfer may be performed either by recovering a portion of the reaction mix from the first amplification reaction and introducing that sample into a second reaction tube or chamber, or by removing a portion of the liquid from the completed first amplification, leaving behind a portion, and adding fresh reagents into the tube in which the first amplification was performed. In either case, additional buffers, polymerase, etc., may then be added in conjunction with the common primers to produce amplified products for detection. The amplification of target molecules using common primers gives a semi-quantitative result wherein the quantitative numbers of targets amplified in the first amplification are amplified using common, rather than target-specific primers—making it possible to produce significantly higher numbers of targets for detection and to determine the relative amounts of the cells comprising various rearrangements within an individual blood sample. Also, combining the second reaction mix with a portion of the first reaction mix allows for higher concentrations of target-specific primers to be added to the first reaction mix, resulting in greater sensitivity in the first amplification reaction. It is the combination of specificity and sensitivity, along with the ability to achieve quantitative results by use of a method such as the arm-PCR method, that allows a sufficiently sensitive and quantitative assessment of the type and number of clonotypes in a population of cells to produce a diversity index that is of diagnostic use.

Clonal expansion due to recognition of antigen results in a larger population of cells that recognize that antigen, and evaluating cells by their relative numbers provides a method for determining whether an antigen exposure has influenced expansion of antibody-producing B cells or receptor-bearing T cells. This is helpful for evaluating whether there may be a particular population of cells that is prevalent in individuals who have been diagnosed with a particular disease, for example, and may be especially helpful in evaluating whether or not a vaccine has achieved the desired immune response in individuals to whom the vaccine has been given.

Primers for amplifying and sequencing variable regions of immune system cells are available commercially, and have been described in publication such as the inventor's published patent applications WO2009137255 and US201000021896A1.

There are several commercially available high-throughput sequencing technologies, such as Hoffman-LaRoche, Inc.'s 454® sequencing system. In the 454® sequencing method, for example, the A and B adaptor are linked onto PCR products either during PCR or ligated on after the PCR reaction. The adaptors are used for amplification and sequencing steps. When done in conjunction with the arm-PCR technique, A and B adaptors may be used as common primers (which are sometimes referred to as “communal primers” or “superprimers”) in the amplification reactions. After A and B adaptors have been physically attached to a sample library (such as PCR amplicons), a single-stranded DNA library is prepared using techniques known to those of skill in the art. The single-stranded DNA library is immobilized onto specifically-designed DNA capture beads. Each bead carries a unique singled-stranded DNA library fragment. The bead-bound library is emulsified with amplification reagents in a water-in-oil mixture, producing microreactors, each containing just one bead with one unique sample-library fragment. Each unique sample library fragment is amplified within its own microreactor, excluding competing or contaminating sequences. Amplification of the entire fragment collection is done in parallel. For each fragment, this results in copy numbers of several million per bead. Subsequently, the emulsion PCR is broken while the amplified fragments remain bound to their specific beads. The clonally amplified fragments are enriched and loaded onto a PicoTiterPlate® device for sequencing. The diameter of the PicoTiterPlate® wells allows for only one bead per well. After addition of sequencing enzymes, the fluidics subsystem of the sequencing instrument flows individual nucleotides in a fixed order across the hundreds of thousands of wells each containing a single bead. Addition of one (or more) nucleotide(s) complementary to the template strand results in a chemilluminescent signal recorded by a CCD camera within the instrument. The combination of signal intensity and positional information generated across the PicoTiterPlate® device allows the software to determine the sequence of more than 1,000,000 individual reads, each is up to about 450 base pairs, with the GS FLX system.

Having obtained the sequences using a quantitative and/or semi-quantitative method, it is then possible to calculate the D50, for example, by determining the percent of clones that account for at least about 50% of the total clones detected in the individual sample. Normal ranges may be compared to the numbers obtained for an individual individual, and the result may be reported both as a number and as a normal or abnormal result. This provides a physician with an additional clinical test for diagnostic purposes. Results for individual samples from a healthy individual, an individual with colon cancer, and an individual with lung cancer are shown below in Table 1. These results are from T-cell populations, expressed as an average of results from 8 (age matched normal) to 10 (colon cancer, lung cancer) samples.

TABLE 1

| Health Condition | D50 (Tc) | D50 (Tr) | D50 (Th) |
|---|---|---|---|
| Healthy/Normal | 23.6 | 43.5 | 38.9 |
| Colon Cancer | 4.5 | 21.7 | 28.3 |
| Lung Cancer | 4.5 | 17.1 | 26.8 |

As each number represents the percent of clones making up about 50 percent of the total number of sequences detected in the population being assessed, it is clear from the numbers above that a lack of immunorepertoire diversity, expressed as a deviation from normal, may be a useful criterion for use in diagnostic test panels. The method of the invention, particularly if used in an automated system such as that described by the inventor in U.S. Patent Application Publication Number 201000291668A1, may be used to analyze samples from multiple individuals, with detection of the amplified targets sequences being accomplished by the use of one or more microarrays.

Hybridization, utilizing at least one microarray, may also be used to determine the D50 of an individual's immunorepertoire. In such a method, the D50 would be calculated as the percentage of the most dominant variable genes (V and/or J genes) which would account for at least 50% of the total signal from all the V and or J genes.

Table 2 illustrates the difference in B-cell diversity, as evidenced by the D50, between (8) normal, healthy individual and (20) individuals with chronic lymphocytic leukemia, and (12) Lupus individuals

TABLE 2

| Individual Condition | D50 (IgH) |
|---|---|
| Healthy/Normal | 95.3 |
| Chronic | 17.86 |
| Lymphocytic | |
| Leukemia | |
| Lupus | 26.5 |

Recently, researchers in various laboratories have reported that microbial diversity within a human or animal (the “microbiome”) also shifts when the healthy state changes to a more unhealthy state. For example, shifts in microbial populations have been associated with various gastrointestinal disorders, with obesity, and with diabetes, for example. Zaura et al. (Zaura, E. et al. “Defining the healthy ‘core microbiome’ of oral microbial communities.” BMC Microbiology (2009) 9: 259) reported that a major proportion of bacterial sequences of unrelated healthy individuals is identical, and the proportion shifts in individuals who have oral disease. The arm-PCR method, combined with high-throughput sequencing, provides a relatively fast, highly sensitive, specific, and semi-quantitative method for evaluating diversity of microbial populations to establish a microbial D50 value, for example, for various human or animal tissues. Arm-PCR has been shown to be quite effective for identifying bacteria within mixed populations obtained from clinical samples.

### Examples

### Individual Samples

Whole blood samples (40 ml) collected in sodium heparin from 10 lung and 10 colon, and 10 breast cancer individuals were purchased from Conversant Healthcare Systems (Huntsville, Ala.). Whole blood samples (40 ml) collected in sodium heparin from 8 normal control samples were purchased from ProMedDx (Norton, Mass.).

### Isolation of T Cell Subsets.

T cell isolations were performed using superparamagnetic polystyrene beads (MiltenyiBiotec) coated with monoclonal antibodies specific for each T cell subset. From whole blood, mononuclear cells were obtained by Ficoll prep, and monocytes removed using anti-CD14 microbeads. This monocyte-depleted mononuclear fraction was then used as a source for specific T cell subset fractions.

Cytotoxic CD8+ T cells were isolated by negative selection using anti-CD4 multisort beads (MiltenyiBiotec), followed by positive selection with anti-CD8 beads. CD4+ T cells were isolated by positive selection with anti-CD4 beads. Anti-CD25 beads (MiltenyiBiotec) were used to select CD4+CD25+ regulatory T cells. All isolated cell populations were immediately resuspended in RNAprotect (Qiagen).

### RNA Extraction and Repertoire Amplification

RNA extraction was performed using the RNeasy Mini Kit (Qiagen) according to the manufacturer's protocol. For each target, a set of nested sequence-specific primers (Forward-out, Fo; Forward-in, Fi; Reverse-out, Ro; and Reverse-in, Ri) was designed using primer software available at www.irepertoire.com. A pair of common sequence tags was linked to all internal primers (Fi and Ri). Once these tag sequences were incorporated into the PCR products in the first few amplification cycles, the exponential phase of the amplification was carried out with a pair of communal primers. In the first round of amplification, only sequence-specific nested primers were used. The nested primers were then removed by exonuclease digestion and the first-round PCR products were used as templates for a second round of amplification by adding communal primers and a mixture of fresh enzyme and dNTP. Each distinct barcode tag was introduced into amplicon from the same sample through PCR primer.

### Sequencing

Barcode tagged amplicon products from different samples were pooled together and loaded into a 2% agarose gel. Following electrophoresis, DNA fragments were purified from DNA band corresponding to 250-500 bp fragments extracted from agarose gel. DNA was sequenced using the 454 GS FLX system with titanium kits (SeqWright, Inc.).

### Sequencing Data Analysis

Sequences for each sample were sorted out according to barcode tag. Following sequence separation, sequence analysis was performed in a manner similar to the approach reported by Wang et al. (Wang C, et al. High throughput sequencing reveals a complex pattern of dynamic interrelationships among human T cell subsets. Proc Natl Acad Sci USA 107(4): 1518-1523). Briefly, germline V and J reference sequences, which were downloaded from the IMGT server (http://www.imgt.org), were mapped onto sequence reads using the program IRmap. The boundaries defining CDR3 region in reference sequences were mirrored onto sequencing reads through mapping information. The enclosed CDR3 regions in sequencing reads were extracted and translated into amino acid sequence.

This application references various publications. The disclosures of these publications, in their entireties, are hereby incorporated by reference into this application to describe more fully the state of the art to which this application pertains. The references disclosed are also individually and specifically incorporated herein by reference for material contained within them that is discussed in the sentence in which the reference is relied on.

The systems, methodologies and the various embodiments thereof described herein are exemplary. Various other embodiments of the systems and methodologies described herein are possible.

Now, therefore, the following is claimed:

1. A method of presenting a user's immunorepertoire profile to the user, comprising the steps of:
obtaining a blood sample from the user;
determining at least one index for selected from the group consisting of the clonotype index, essential index, and diversity index to produce an immunorepertoire profile for the blood sample for the user; and
outputting information to the user pertaining to the user's immunorepertoire profile.

2. The method of claim 1, further comprising the step of obtaining a set of characteristic data associated with the user, wherein the characteristic data associated with the user comprises the user's age and gender.

3. The method of claim 2, wherein the characteristic data further comprises the presence of any disease.

4. The method of claim 1, wherein the blood sample comprises whole blood.

5. The method of claim 1, wherein the blood sample comprises a dried blood spot.

6. The method of claim 5, comprising the additional steps of:
providing the user with a kit comprising a blood collection card, wherein the blood collection card comprises at least one blood collection area and a QR code; and
scanning the QR code by the user to associate the blood sample with the user's account on a software application.

7. The method of claim 1, wherein the step of outputting information to the user is performed using a software application.

8. A method of presenting a user's immunorepertoire profile to the user, comprising the steps of:
providing the user with a kit comprising a blood collection card, wherein the blood collection card comprises at least one blood collection area and a QR code;
scanning the QR code by the user to associate the blood sample with the user's account on a software application;
obtaining a set of characteristic data associated with the user, wherein the characteristic data associated with the user comprises the user's age, gender and the presence or absence of any disease;
obtaining a blood sample from the user;
determining at least one index for selected from the group consisting of the clonotype index, essential index, and diversity index to produce an immunorepertoire profile for the blood sample for the user; and
outputting information to the user pertaining to the user's immunorepertoire profile using a software application.
